(19) United States
(12) Patent Application Publication
(10) Pub. No.: US 2009/0162290 A1
(43) Pub. Date: Jun. 25, 2009

(54) METHOD FOR THE SYNTHESIS OF PENTA-PENDANT ENANTIOMER-PURE CHELATORS AND PROCESS FOR THERAPEUTICALLY ACTIVE BIOCONJUGATES PREPARATION BY A COVALENT BINDING THEREOF

(75) Inventors: Ivan Benes, Forch (CH); Simon Cihelnik, Krupka (CZ); Ladislav Droz, Kladno (CZ); Martin Sramek, Kladno (CZ)

Correspondence Address: URSULA B. DAY, ESQ., 708 Third Avenue, SUITE 1501, NEW YORK, NY, 10017, US

(73) Assignee: THERAPHARM GmbH

(21) Appl. No.: 12/066,343

(22) PCT Filed: Sep. 11, 2006
(86) PCT No.: PCT/EP2006/008789

§ 371 Date: Jan. 7, 2009

(30) Foreign Application Priority Data
Sep. 9, 2005 (EP) 05019671.6

(51) Int. Cl. A61K 49/16; C07K 16/00

(52) U.S. Cl. 424/9.34; 530/391.3

(57) ABSTRACT

The present invention provides a method for synthesis and binding methods of pentapendant enantiomer-pure chelators of formula (VII) wherein R₁, R₂, R₃, R₄ are groups forming an adequate enantiomer of the chelator; and X₁-X₅, Y₁-Y₅, Z₁-Z₅ each individually forming pendant chelating groups.

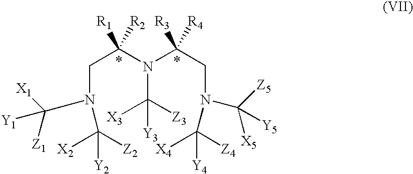

### BACKGROUND OF INVENTION

Functionalized specific ligands for a metal cation binding are widely studied group of molecules (M. Woods e.a. Chimica Oggy 2005, 31). Possibilities of selective, fixed and fast cation complexations on the one hand, and biological or an analytical active molecule binding on the other hand are priceless in end applications. There are two main ways of application: a radiopharmaceutical, with complexated cation of a radionuclide (S. Liu Bioconjugate Chem. 2001, 12, 7), and a spectroscopical, with spectroscopically active complexated cation or a bound analytical molecule.

An increasing therapeutic application of radiopharmaceuticals in human medicine is made possible by an availability of specific nuclide carriers. In case of a cation nuclide as radioisotope, specific ligands (also called chelators, complexanes, ionophores etc.) are a crucial structural fragment of the radiopharmaceutics. A stability and complexing specificity of a complexated radionuclide is a key of a radionuclide toxicity rejection in action stage of a radiopharmaceutic.

Also important is following: when a biological address is bound to the structure of a ligand, a progressive targeted therapeutic is created. Targeted therapeutics of this idea decreased total organism stress during a radiotherapy.

An application range of radiopharmaceuticals is wide. Besides extremely perspective tumor invasive therapy (H. M. Vriesendor e.a. BioDrugs 1998, 10(4), 275; S. M. Quadri e.a. J. Nucl. Med. 1996, 37(9), 1545), there are numerous applications in a cancer or an inflammatory diagnosis (NMR tomography, scintillation cameras) and also organ or tissue metabolic studies. Typical isotopes for a radiotherapeutical use are ⁹⁰Y, ¹¹¹In, Gd etc.

There are two basic requirements for parameters of a ligand derived from a chemical structure: 1. High thermodynamic stability of the complex (in vivo), high selectivity for the complexated cation in the applied milieu (in vivo) and the fast complexation with complexated cation (in vitro). 2. No metabolic process possibility of ligand in an action stage (in vivo), total and the fast elimination of ligand from organism in an after-action stage (in vivo).

In radiotherapeutical applications are widely used diethylenetriaminepentaacetic acid (DTPA) derivatives as efficient ligands.

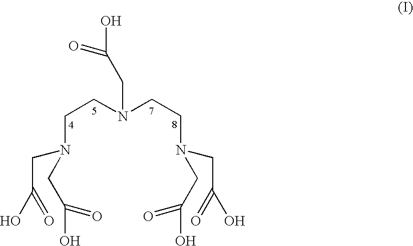

Free DTPA (I) is not suitable for that idea due to no possibility of a biological molecule covalent binding. Therefore the preparation of functionalized derivatives of DTPA was started. From studied derivatives, a well-flipped 4-aminobenzyl group binded to skeleton of DTPA (II) satisfies all needs and it brings important properties into the backbone. Namely, the well-flipped methylene bridge spaced 4-aminophenyl can support all complexation effects (rate and efficiency) as well as optimal length of 4-aminobenzyl excepts a possibility of damaging interaction by a binded biologically active substrate with the backbone of the ligand.

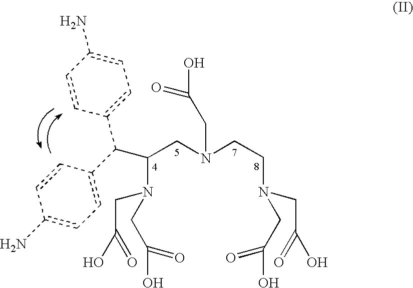

Each C-substitution to the backbone of DTPA brings one stereogenic center. Similarly in case of a 4-aminobenzyl substituted DTPA (III), (IV). Independently on that 4-aminobenzyl substituent position in DTPA skeleton there are present structural fragments of 2-alkyl-2-aminoethylbenzene

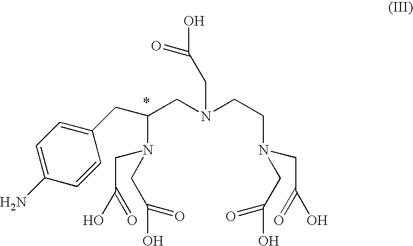

derivatives as two possible isomers (R or S). Thus, strong internalization metabolism dependence on the present isomer is evident. And therefore enantiomer pure DTPA derivatives are necessary for obtaining therapeutically defined and optimal ligand parameters.

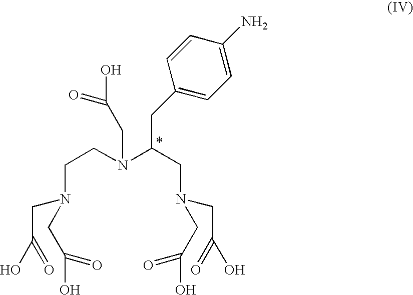

There are only few published studies inquired in an evaluation of those application parameters referenced to structural characteristics of the DTPA ligands. McMurry (J. Med. Chem. 1998, 41, 3546) in basic work compares eight derivatives of DTPA with unsubstituted DTPA. That group contains a four 4-nitrobenzyl, methyl substituted DTPA derivatives and three cyclohexano condensed DTPA ligands with a defined conformation. He obtained a set of interesting results. He showed that each C-substitution on DTPA skeleton increases the rate of an Yttrium complex formation. The best stability constants and the lowest dissociation rates be obtained in case of two 4-nitrobenzyl-cyclohexano derivatives. Group of 4-nitrobenzyl cyclohexano DTPA analogs was also extensively studied by Wu (Radiochimica Acta 1997, 79, 123; Bioorganic & Medicinal Chemistry 1997, 5, 1925). There was used ⁸⁸Y for complexation in the first case and there were further studied stereochemical influences on stability of radiometal complexes in vivo.

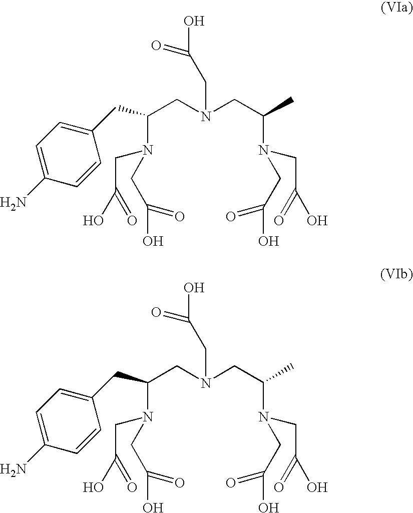

US 2004/0208828 (L. Lehmann e.a.) summarizes differences between defined conformations of 4-(4-nitrobenzyl)-8-methyl DTPA derivatives. It was shown that diastereoisomer mixture (V) has lower constant stability of Yttrium complex than appropriate isomers (VIa) or (VIb). Some few descriptions of a marginal 5,7-substituted DTPA or its isomer properties are only available in literature. Enantiomer undefined 5-(4-nitrobenzyl)-7-methyl DTPA synthesis is described in literature (S. M. Quadri e.a. Bioorg. Med. Chem. Lett. 1992, 2(12), 1661).

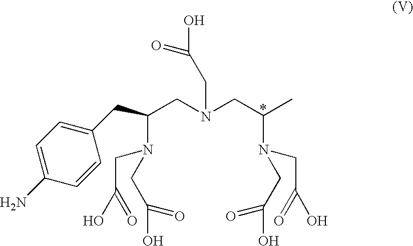

A process for preparing of a DTPA 4-benzyl-7,8-substituted derivatives is described in U.S. Pat. No. 6,207,858 (P. Chinn e.a.) and DTPA 4-benzyl-8-substituted derivatives are described by Cummins (Bioconjugates Chem. 1991, 2, 180). Nevertheless, no pure enantiomer 7,8-substituted DTPA derivative was obtained. Starting molecule was 4-nitro-L-phenylalanine; therefore S conformation of 4-benzyl substituent was possible to declare.

Brechbiel (J. Chem. Soc., Perkin Trans I 1992, 1173) describes some rigid C-functionalized DTPA of cyclohexano type for labeling of monoclonal antibodies with the ²¹²Bi. Similar molecules were synthesized by Sun (Inorg. Chem. 2000, 39, 1480) as racemic and meso forms and ligands were applied for a complexation study of Gd. Synthesis of other important DTPA derivatives was published by Chong (J. Org. Chem. 2001, 66(23), 7745) and by Laureat (Magnetic Resonance Materials in Physics, Biology and Medicine 2004, 16, 235).

Due to a high importance of ligands enantiomer purity, a lot of works describe methods for enantiomer pure DTPA derivatives synthesis. Thus, conformationally constrained DTPA analogues from L- or D-serine and trans-4-hydroxy-L-proline were synthesized by Pickersgill (J. Org. Chem. 2000, 65(13), 4048). Grote (J. Org. Chem. 1995, 60(21), 6987) published stereocontrolled synthesis of DTPA analogs; Williams (J. Org. Chem. 1994, 59(13), 3616-25) synthesized aminopyrrolidine analogs of DTPA and enantiomer pure DTPA derivatives started from L-phenylalanine (J. Org. Chem. 1993, 58(5), 1151).

A biodistribution of ¹¹¹In or ⁸⁸Y complexated bioconjugates of 4-(4-isothiocyanatobenzyl)-8-methyl DTPA was studied in detail by Camera (Eur. J. Nucl. Med. 1994, 21, 640).

### DETAILED DESCRIPTION OF INVENTION

As already described, enantiomer pure chelators have considerably better complexing properties and strictly defined metabolism than appropriate diastereomer mixtures or general isomer mixtures. Due to a characteristic strong rigid configuration on terminal carbons of the central amino group, the compounds according to the invention show strictly defined space configuration. This effect induces efficient and fast complexations with minimized influences of an application milieu nature.

The compounds according to the invention have strong hydrophilic character. Therefore these compounds show excellent solubilization properties in aqueous systems, which is the important parameter in all expected application fields (tissue studies, radiotherapy, radiodiagnosis, NMR tomography etc.). By the substitution effect, the compounds according to the invention afford large possibilities of dissociation constants modulation. This takes effect in metabolic stability of complexated ligands according to the invention, above all in kidney.

Thus, the present invention describes the pentapendant enantiomer pure chelators of the formula (VII)

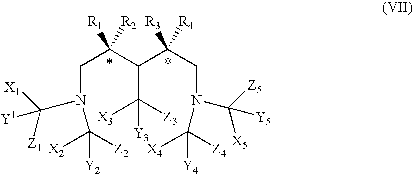

wherein

X₁-X₅, Y₁-Y₅, Z₁-Z₅ are each individually hydrogen, substituted or unsubstituted C₁-C₂₄ alkyl, C₂-C₂₄ alkenyl or cycloalkyl, substituted or unsubstituted aryl or heteroaryl, especially O-substituted or unsubstituted carboxyl, nitrile, N-substituted or unsubstituted carboxamide, formyl, N-hydroxyiminomethyl, independently O- and N-substituted or unsubstituted N-hydroxyaminocarbonyl, phosphonyl, phosphinyl, alkylphosphonyl, alkylphosphonyl, arylphosphonyl, arylphosphonyl forming pendants, wherein alkyl, alkenyl and cycloalkyl may be substituted with e.g. aryl, halogen, hydroxyl, C₁-C₁₂ alkoxy, oxo, carboxyl, carboxy-C₁-C₁₂alkyl, nitrile, amino and/or carboxamide, wherein aryl may be substituted e.g. with C₁-C₁₂ alkyl, halogen, hydroxyl, C₁-C₁₂ alkoxy, carboxyl, carboxy-C₁₋₁₂ alkyl, nitrile, amino and/or carboxamide, and wherein carboxyl and carboxamide and N-hydroxyaminocarbonyl may be substituted, e.g. with C₁-C₁₂ alkyl;

R₁, R₂, R₃, R₄ are groups forming an adequate enantiomer (R,R), (R,S), (S,R) or (S,S). R₁, R₂, R₃, R₄ are independently hydrogen, substituted or unsubstituted C₁-C₂₄ alkyl, C₂-C₂₄ alkenyl or cycloalkyl or substituted or unsubstituted aryl or heteroaryl wherein preferred substituents are as defined above. Groups R₁, R₂, R₃ and R₄ are preferably selected such that R₁ is different from R₂ and R₃ is different from R₄. At least one of R₁, R₂, R₃ and R₄ is especially C₁-C₄-alkyl-aryl, e.g. a 4-substituted benzyl of the structure (VIII).

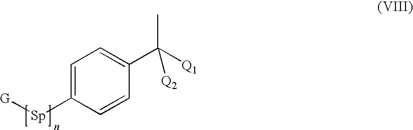

wherein

Q₁, Q₂ are each individually hydrogen, substituted or unsubstituted C₁-C₂₄ alkyl, substituted or unsubstituted aryl or heteroaryl, substituted or unsubstituted carboxyl, or N-substituted or unsubstituted carboxamide; wherein alkyl may be substituted with e.g. aryl halogen, hydroxyl, C₁-C₁₂ alkoxy, oxo, carboxyl, carboxy-C₁-C₁₂alkyl, nitrile, amino and/or carboxamide, wherein aryl or heteroaryl may be substituted with e.g. C₁-C₁₂ alkyl, halogen, hydroxyl, C₁-C₁₂ alkoxy, carboxyl, carboxy-C₁₋₁₂ alkyl, nitrile, amino and/or carboxamide, and wherein carboxyl and carboxamide may be substituted e.g. with C₁-C₁₂ alkyl.

Sp is spacing group of the formula

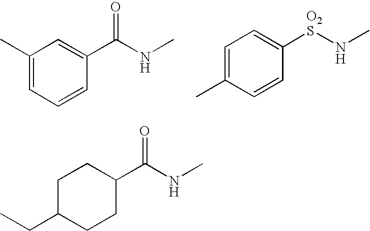

n is 0 or 1;

G is hydrogen, substituted or unsubstituted C₁-C₂₄ alkyl or C₂-C₂₄ alkenyl, N-substituted or unsubstituted amine, N-substituted or unsubstituted hydrazine, hydroxyl, O-alkylhydroxyl, O-acylhydroxyl, thiol, S-alkylthiol, S-substituted disulfide, O-substituted or unsubstituted carboxyl, N-substituted or unsubstituted carboxamide, isocyanate, isothiocyanate, carboxamidine, carbohydrazide, nitro, nitroso, formyl, formyl forming cyclic or uncyclic acetal, acetyl, 2-haloacetyl, halomethyl, hydroxymethyl or dihydroxyboronyl, wherein preferred substituents are as indicated above, and wherein hydrazine and disulfide may be substituted with e.g. C₁-C₂₄ alkyl,

or is a linker of the formula

A

or

B

or

C

or

A-B-(C)_{α}

or

A₁-B-A₂-(C)_{α}

or

A₁-A₂-A₃-(C)_{α}

or

A₁-A₂-A₃-A₄-(C)_{α}

or

A₁-(A)_{β}-A₃-(C)_{α}

or

A₁-B₁-(A₂-B₂)_{γ}-A₃-B₃-(C)_{α}

wherein β, γ are each individually from 0 to 24; α is 0 or 1; wherein A₁, A₂, A₃, A₄ are independently fragments of structure A; B₁, B₂ are independently fragments of structure B;
wherein A is fragment of structure (IX)

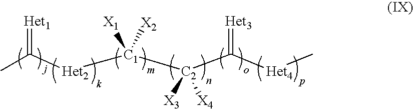

wherein j, k, m, n, o, p are each individually from 0 to 12; Het₁-Het₄ are independently O, S, NR_{Het}, wherein R_{Het} is hydrogen, substituted or unsubstituted C₁-C₁₂ alkyl, substituted or unsubstituted aryl; X₁-X₄ are each individually hydrogen, substituted or unsubstituted primary C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl or X₁-X₄ can form mutually 5-membered and 6-membered saturated or unsaturated cycles, aromatic cycles and heterocycles; or X₁-X₄ can form mutually and each individually an oxo group or a double and triple bond between C₁ and C₂; wherein preferred substituents are as indicated above; and wherein amine may be substituted with C₁-C₁₂ alkyl or C₁-C₁₂ alkoxy;
wherein B is fragment of structure (X)

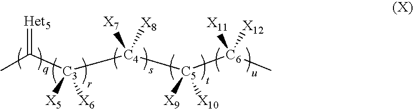

wherein q, r, s, t, u are each individually from 0 to 12; Het₅ is independently O, S, NR_{Het}, wherein R_{Het} is hydrogen, substituted or unsubstituted C₁-C₁₂ alkyl, substituted or unsubstituted aryl; X₅-X₁₂ are each individually hydrogen, substituted or unsubstituted primary C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl; or X₅-X₁₂ can form mutually 5-membered and 6-membered saturated or unsaturated cycles, aromatic cycles and heterocycles or X₅-X₁₂ can form mutually and each individually an oxo group or one or two double and triple bonds between C₁, C₂, C₃ or C₄, wherein preferred substituents are as indicated above.

C is a reactive group, particularly a structural fragment selected from the group of hydroxyl, carboxyl, amino group, chloroacetyl, bromoacetyl group, iodoacetyl group, carbonyl chloride, carbonyl fluoride, carbonyl bromide, sulphonyl chloride, sulphonyl fluoride, sulphonyl bromide, sulphonyl arylsulphonate, sulphonyl alkylsulphonate, or an active ester, e.g. selected from the group:

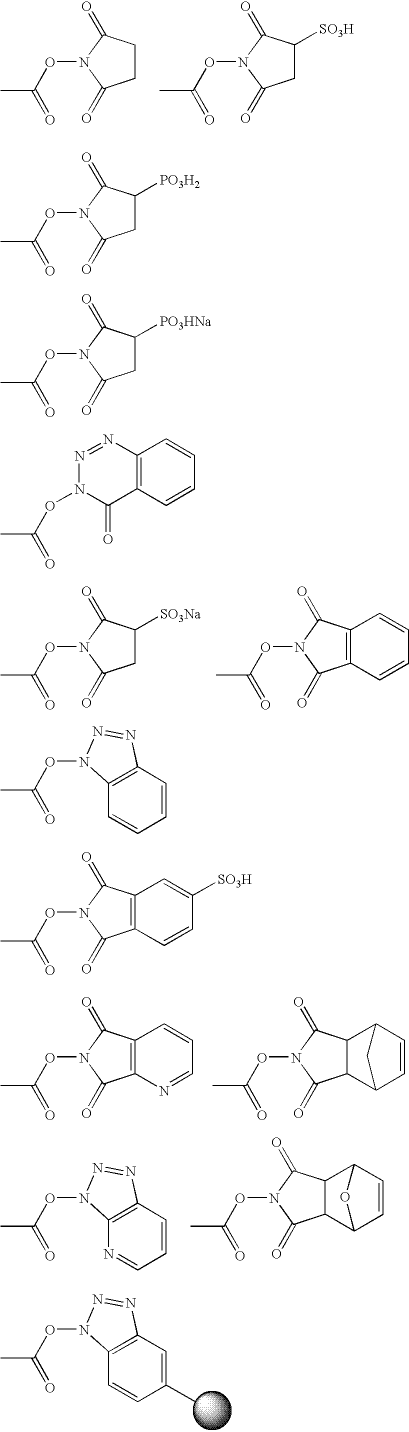

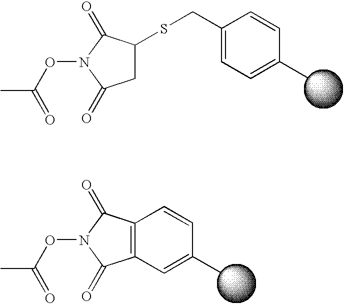

or from the group:

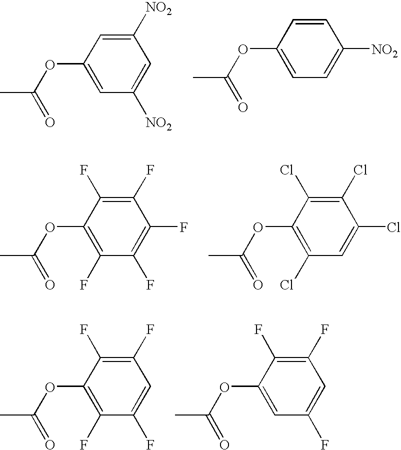

or a biologically active molecule, especially a biopolymer. The biologically active molecule may be a natural substrate present in an organism or its synthetic analog. Preferably, the molecule has biologic activity in a physiological function, especially in metabolic effect control or reproduction. The biopolymer may be selected from polypeptides, saccharides, or nucleic acids and it often comprises amino acids, monosaccharides, nucleobases and/or fatty acids. The biomolecules are especially selected from this group:

antibodies, e.g. monoclonal antibodies (e.g. antiCD33, antiCD25, antiCD66), antibody fragments, polyclonal antibodies, minibodies, DNA and RNA fragments, such as derivatized DNAs and RNAs, synthetic RNA and DNA (also with unnatural bases), virus and retrovirus fragments, hormones, cytokines, lymphokines such as HGH (human growth hormone, somatotropin), somatostatin and derivatives thereof, IGF-1 (somatomedin) and derivatives thereof, IGF-2, IGF-protein-3, somatostatin-biotin derivatives, tumor-specific proteins and synthetic agents, vascular endothelial growth factor, myoglobins, apomyoglobins, neurotransmitter peptides, octreotide, lanreotide, Somatuline, vapreotide, tumor necrosis factors, peptides that accumulate in inflamed tissues, blood-pool reagents, anion- and cation-transporter proteins, red blood corpuscles and other blood components, cancer markers and cell adhesion substances, peptides that can be cleaved by proteases, peptides with predetermined synthetic sites of rupture, peptides that are cleaved by metalloproteases, peptides with photocleavable linkers, peptides with oxidative agents and cleavable groups, peptides with natural and unnatural amino acids, glycoproteins (glycopeptides), signal proteins, antiviral proteins and apoptosis proteins, proteins and peptides, which accumulate at certain spots in the organism, neuramidases, neuropeptides, immunomodulators, endoglycosidases, substrates that are activated by enzymes such as calmodulin kinase, caseinkinase 11, glutathione-S-transferase, heparinase, matrix-metalloproteases, O-insulin-receptor-kinase, UDP-galactose 4-epimerase, fucosidases, G-proteins, galactosidases, glycosidases, glycosyltransferases and xylosidase, carbohydrates (mono- to polysaccharides), such as derivatized sugars, sugars that can be cleaved in the organism, cyclodextrins and derivatives thereof, amino sugars, chitosan, polysulfates and acetylneuraminic acid derivatives, steroids (natural and modified), hormones, antihormones, bioactive lipids, fats, fatty acid esters, synthetically modified mono-, di- and triglycerides, liposomes, which are derivatized on the surface, micelles that consist of natural fatty acids or perfluoroalkyl compounds, nucleosides, nucleotides, porphyrins, texaphrines, expanded porphyrins, cytochromes, inhibitors, synthetically modified biopolymers, such as biopolymers that are derivatized with linkers, synthetic polymers, which are directed to a biological target (e.g. receptor), polymers that accumulate in acidic or basic areas of the body (pH-controlled dispersion).

New synthetic methods for an enantiomer pure derivatives of the structure (VII) are provided.

The compounds according to the invention can be synthesized based on reaction of enantiomer-pure amine of the structure (XI)

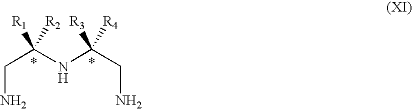

wherein

R₁, R₂, R₃, R₄ are groups forming an adequate enantiomer (R,R), (R,S), (S,R) or (S,S). R₁, R₂, R₃, R₄ are independently hydrogen, substituted or unsubstituted C₁-C₂₄ alkyl, C₂-C₂₄ alkenyl or cycloalkyl, substituted or unsubstituted aryl or heteroaryl, wherein preferred substituents are as indicated above, especially a 4-substituted benzyl of the structure (VIII)

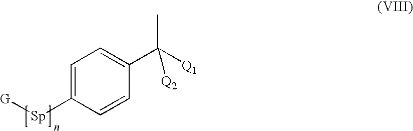

wherein

Q₁, Q₂ are each individually hydrogen, substituted or unsubstituted C₁-C₂₄ alkyl, substituted or unsubstituted aryl or heteroaryl, substituted or unsubstituted carboxyl or N-substituted or unsubstituted carboxamide; wherein preferred substituents are as indicated above,

Sp is the spacing group of the formula

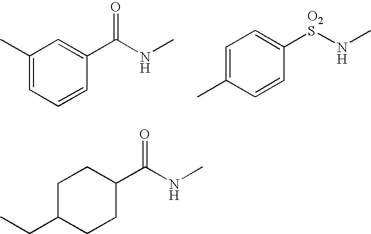

n is 0 or 1;

G is hydrogen, C₁-C₂₄ alkyl, C₂-C₂₄ alkenyl, N-substituted or unsubstituted amine, N-substituted or unsubstituted hydrazine, hydroxyl, O-alkylhydroxyl, O-acylhydroxyl, thiol, S-alkylthiol, O-substituted or unsubstituted carboxyl, N-substituted or unsubstituted carboxamide, isocyanate, isothiocyanate, carboxamidine, carbohydrazide, nitro, nitroso, formyl, formyl forming cyclic or uncyclic acetal, acetyl, 2-haloacetyl, halomethyl, hydroxymethyl or dihydroxyboronyl; wherein preferred substituents are as indicated above,

or is a linker of the formula

A

or

B

or

C

or

A-B-(C)_{α}

or

A₁-B-A₂-(C)_{α}

or

A₁-A₂-A₃-(C)_{α}

or

A₁-A₂-A₃-A₄-(C)_{α}

or

A₁-(A)_{β}-A₃-(C)_{α}

or

A₁-B₁-(A₂-B₂)_{γ}-A₃-B₃-(C)_{α}

wherein β, γ are each individually from 0 to 24; α is 0 or 1; wherein A₁, A₂, A₃, A₄ are independently fragments of structure A; B₁, B₂ are independently fragments of structure B;
wherein A is a fragment of structure (IX)

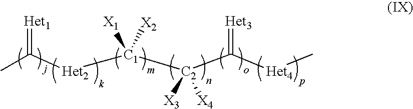

wherein j, k, m, n, o, p are each individually from 0 to 12; Het₁-Het₄ are independently O, S, NR_{Het}, wherein R_{Het} is hydrogen, substituted or unsubstituted C₁-C₁₂ alkyl, substituted or unsubstituted aryl; wherein preferred substituents are as indicated above;

X₁-X₄ are each individually hydrogen, substituted or unsubstituted primary C₁-C₁₂ alkyl or cycloalkyl, substituted or unsubstituted aryl, hydroxyl, alkoxyl, aryloxyl, halogen, substituted or unsubstituted amine, carboxyl, N-substituted or unsubstituted carboxamide, nitrile, alkoxycarbonyl; wherein preferred substituents are as indicated above;

or X₁-X₄ can form mutually 5-membered and 6-membered saturated or unsaturated cycles, aromatic cycles and heterocycles; or X₁-X₄ can form mutually and each individually an oxo group, or a double and triple bond between C₁ and C₂;
wherein B is a fragment of structure (X)

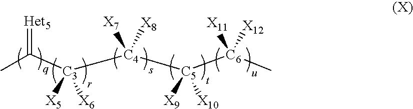

wherein q, r, s, t, u are each individually from 0 to 12; Het₅ is independently O, S, NR_{Het}, wherein R_{Het} is hydrogen, substituted or unsubstituted C₁-C₁₂ alkyl, substituted or unsubstituted aryl; X₅-X₁₂ are each individually hydrogen, substituted or unsubstituted primary C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl or X₅-X₁₂ can form mutually 5-membered and 6-membered saturated or unsaturated cycles, aromatic cycles and heterocycles; or X₅-X₁₂ can form mutually and each individually an oxo group, or one or two double and triple bonds between C₁, C₂, C₃ or C₄, wherein preferred substituents are as indicated above;

C may be a reactive group, particularly a structural fragment selected from the group of hydroxyl, carboxyl, amino group, chloroacetyl, bromoacetyl group, iodoacetyl group, carbonyl chloride, carbonyl fluoride, carbonyl bromide, sulphonyl chloride, sulphonyl fluoride, sulphonyl bromide, sulphonyl arylsulphonate, sulphonyl alkylsulphonate, or an active ester, e.g. selected from the group:

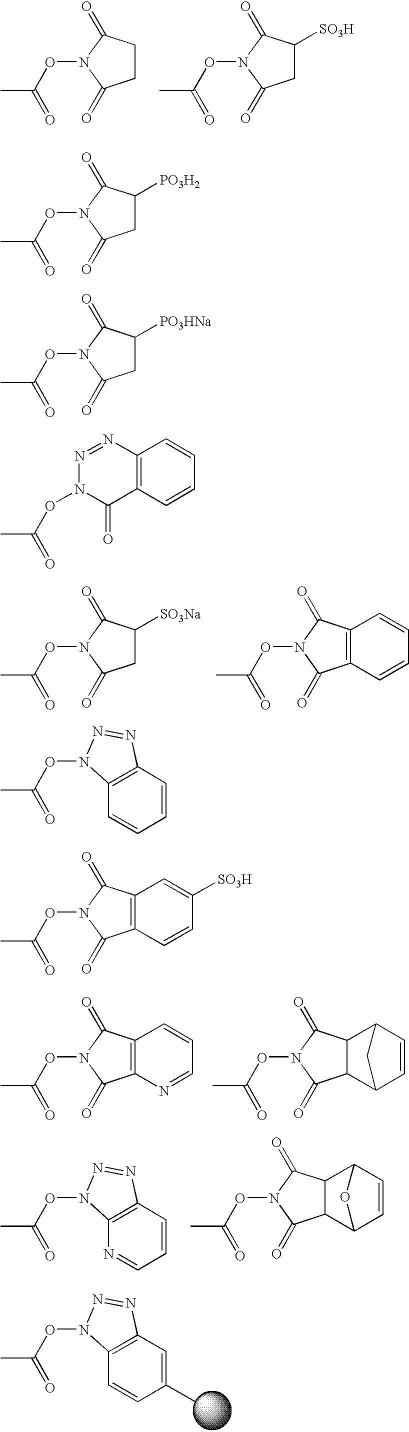

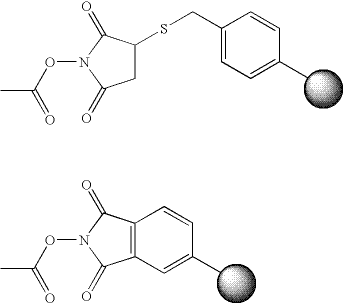

or from the group:

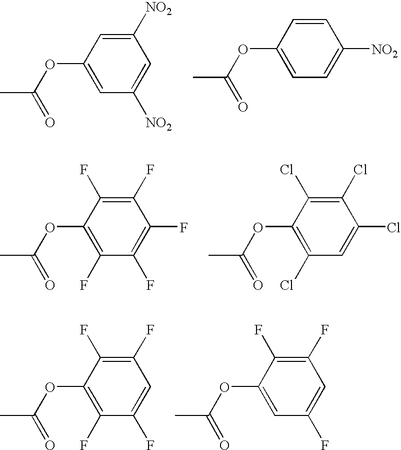

or a biologically active molecule as defined above.

The compound (XI) may be reacted with a carboxyalkylation agent or with a phosphonoalkylation agent or with a phosphinoalkylation agent of the structure (XII)

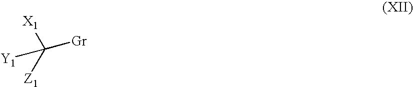

wherein

X₁-X₅, Y₁-Y₅, Z₁-Z₅ are each individually hydrogen, substituted or unsubstituted C₁-C₂₄ alkyl, C₂-C₂₄ alkenyl or cycloalkyl, substituted or unsubstituted aryl or heteroaryl, especially O-substituted or unsubstituted carboxyl, nitrile, N-substituted or unsubstituted carboxamide, formyl, N-hydroxyiminomethyl, alkoxycarbonyl, aryloxycarbonyl, independently O- and N-substituted or unsubstituted N-hydroxyaminocarbonyl, phosphonyl, phosphinyl, alkylphosphonyl, alkylphosphonyl, arylphosphonyl, arylphosphonyl and just one or two substituents from X₁-X₅, Y₁-Y₅, Z₁-Z₅ are each individually carboxyl, nitrile, N-substituted or unsubstituted carboxamide, formyl, alkoxycarbonyl, aryloxycarbonyl, N-hydroxyiminomethyl or independently O- and N-substituted or unsubstituted N-hydroxyaminocarbonyl, phosphonyl, phosphinyl, alkylphosphonyl, alkylphosphonyl, arylphosphonyl or arylphosphonyl, wherein preferred substituents are as indicated above;

Gr may be halogen, hydroxyl, alkoxyl, aryloxyl, oxonium, substituted or unsubstituted amine, substituted or unsubstituted ammonium, sulphonyl, sulphonyloxy, O-acyloxyl, arylsulphonyloxy, halogen, especially bromine, chlorine, iodine, tosyloxy, mesyloxy, triflyloxy, benzoyloxy, methoxycarbonyloxy, perfluoracetyloxy, trimethylammonium, diethyloxonium, 1-benztriazolyloxyl, trialkylsilyloxyl, benzyloxycarbonyloxy, tert.butyloxycarbonyloxyl, N-phthalimidyloxy, 1-imidazolyloxy, N-succinimidyloxyl, N-phthalimidyloxy, wherein preferred substituents are as indicated above;

The agent (XII) may also be generated in situ from a two- or three-part reaction system, e.g. from hydrogen cyanide and formaldehyde; alkaline cyanide, formaldehyde and a mineral acid; formaldehyde and methyl(4-nitrobenzyl)oxophosphorane; formaldehyde and methylphosphinic acid; formaldehyde and diethyl phosphonate; formaldehyde diethylacetal and 4,5-diphenyl-1,3,2λ⁵-dioxaphospholan-2-one.

The reaction conditions preferably comprise conditions of general nucleophilic substitution, especially under conditions of phase-transfer catalysis, e.g. in aprotic polar solvents or mixtures thereof (as dimethylformamide or dimethylacetamide or acetonitrile, dimethylsulphoxide or sulpholane or hexamethylphosphortriamide) or mixtures with at least one protic solvent, e.g. in a micellar medium, in solid-phase (for example with bonded amine (V) on anex), with or without microwave irradiation, with or without ultrasonic irradiation, under conditions of high pressure (for example in autoclave), in aqueous or nonaqueous phase in presence of a pH-buffer, in milieu of water-free solvents with or without presence of a base (e.g. amines, aldimines, carbonates, fluorides, thioethers), especially a strong base with low nucleophily (e.g. N-ethyl-N,N-diisopropylamine (Hüning's base), N-methyl-N,N-dicyclohexylamine, N-methyl-N,N-diisopropylamine, N,N,N′,N′-tetramethyl-1,8-naphtalenediamine), with enzymatic catalysis, in presence of a dehydrating an agent or agent reacting with protogenic product reaction or in presence of a Lewis acid (e.g. ZnCl₂, BF₃.Et₂O, SiCl₄).

A process for the production of compounds according to this description is performed in large temperature range of −78° C.-325° C. with advantage in low temperatures of a range 40-70° C. Mild reaction conditions positively increase purity and enantiomer purity in some cases. The process for production of compounds according to this description is carried out from a short period of seconds to long periods of ten days.

Carboxymethylations as well as phosphonoalkylations or phosphinoalkylations according to this description need efficient alkylation systems to high conversion and steric-protected central amine-nitrogen alkylation.

Thus, e.g. a strong carboxymethylation system, as tert.-butyl iodoacetate-N-methyl-N,N-diisopropylamine in milieu of dimethylformamide, is successfully usable. A role of a base in the reaction is crucial. Potassium carbonate gives lower yields, as well as cesium fluoride, a very mild base. In contrast to carbonate base, carboxylate systems in aqueous or polar aprotic solvents afford very high yields of percarboxymethylated products. Influence of associated counter ion is strong. Low diameter cations with higher surface density of change are preferred. Thus lithium or calcium salts afford the bests yields. A template effect of carboxymethylations in these cases is evident.

Enantiomers are obtained via alkylation methods with no possibility to change a configuration. Therefore, it is necessary to have pure amine (XI) isomer. If a diastereomer mixture is used, a separation of isomers is required. This can be done by diastereomer separation on a chiral column, less preferred on a standard unchiral column, or by recrystallization of diastereomers with an added chiral molecule (e.g. (+)-dehydroabietylamine). Because there is no usable catalyst for an asymmetric catalysis of this type N-alkylation, only increasing of yields it is possible to get.

If carboxylic esters of (VII) are obtained, a hydrolysis is necessary step. tert.-butyl esters are hydrolyzed under mild conditions of acidic cleavage at low temperatures. Thus tert.-butyl esters are suitable, if temperature sensitive groups are coupled to the backbone of (VII). Benzyl esters also need a next deprotection, for example hydrogenolysis, with an advantage carried out by hydrazine in presence of 10% palladium on charcoal.

The compounds according to the invention also can be synthesized based on reaction of (VII)

wherein

R₁, R₂, R₃, R₄ are groups forming an adequate enantiomer (R,R), (R,S), (S,R) or (S,S), wherein R₁, R₂, R₃, R₄ are independently hydrogen, substituted or unsubstituted C₁-C₂₄ alkyl, C₂-C₂₄ alkenyl or cycloalkyl, substituted or unsubstituted aryl or heteroaryl, wherein preferred substituents are as indicated above; especially C₁-C₄ alkyl-aryl, e.g. 4-substituted benzyl of the structure (VIII)

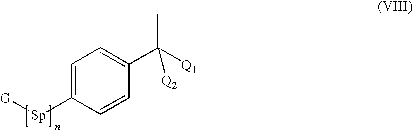

wherein

Q₁, Q₂ are each individually hydrogen, substituted or unsubstituted C₁-C₂₄ alkyl, substituted or unsubstituted aryl or heteroaryl, substituted or unsubstituted carboxyl, N-substituted or unsubstituted carboxamide; wherein preferred substituents are as indicated above;

Sp is spacing group of the formula

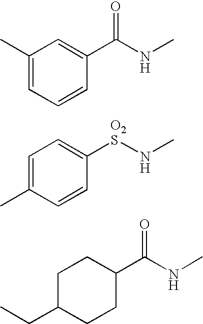

n is 0 or 1;

G is forming a linker of the formula

A-B-(C)_{α}

or

A₁-B-A₂-(C)_{α}

or

A₁-A₂-A₃-(C)_{α}

or

A₁-A₂-A₃-A₄-(C)_{α}

or

A₁-(A)_{β}-A₃-(C)_{α}

or

A₁-B₁-(A₂-B₂)_{γ}-A₃-B₃-(C)_{α}

wherein β, γ are each individually from 0 to 24; α is 1; wherein A₁, A₂, A₃, A₄ are independently fragments of structure A; B₁, B₂ are independently fragments of structure B;
wherein A is fragment of structure (IX)

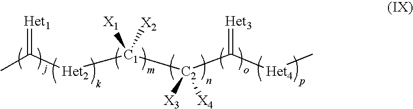

wherein j, k, m, n, o, p are each individually from 0 to 12; Het₁-Het₄ are independently O, S, NR_{Het}, wherein R_{Het} is hydrogen, substituted or unsubstituted aryl or C₁-C₁₂ alkyl, wherein preferred substituents are as indicated above;

X₁-X₄ are each individually hydrogen, substituted or unsubstituted primary C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl or X₁-X₄ can form mutually 5-membered and 6-membered saturated or unsaturated cycles, aromatic cycles and heterocycles; or X₁-X₄ can form mutually and each individually an oxo group, or a double and triple bond between C₁ and C₂; wherein preferred substituents are as indicated above;

wherein B is fragment of structure (X)

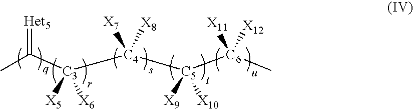

wherein q, r, s, t, u are each individually from 0 to 12; Het₅ is independently O, S, NR_{Het}, wherein R_{Het} is hydrogen, substituted or unsubstituted C₁-C₁₂ alkyl, substituted or unsubstituted aryl; X₅-X₁₂ are each individually hydrogen, substituted or unsubstituted primary C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl or X₅-X₁₂ can form mutually 5-membered and 6-membered saturated or unsaturated cycles, aromatic cycles and heterocycles; or X₅-X₁₂ can form mutually and each individually an oxo group, or one or two double and triple bonds between C₁, C₂, C₃ or C₄, wherein preferred substituents are as indicated above;
and wherein C is a reactive group as indicated above, with a biologically active molecule, especially a biopolymer, as indicated above, by covalent binding.

Methods for bioconjugate preparations are generally known and very well described. Based on the binding center of biologically active molecule, an adequate structural fragment of the enantiomer pure ligand is used for this conjugation process. For example, available primary amino groups, e.g. lysine based strong aliphatic amino groups can be conjugated with e.g. bromoacetyl groups or thiocyanates. Alternatively, thiol groups can be conjugated with appropriate reagents such as maleimides. There are used moderate conditions generally, such as pH buffered aqueous solutions. Organic solvents can be also used, if necessary.

In the compounds as indicated above, the term “alkyl” means C₁-C₂₄ alkyl, preferably C₁-C₁₂ alkyl and more preferably C₁-C₆ alkyl. The term “alkenyl” means C₂-C₂₄ alkenyl, preferably C₂-C₁₂ alkyl and more preferably C₂-C₆ alkenyl. The term “aryl” means preferably C₆-C₁₄ aryl and more preferably C₆-C₁₀ aryl. The term heteroaryl preferably means C₅-C₁₄ heteroaryl and more preferably C₅-C₁₀ heteroaryl and includes N-, O- and/or S-containing rings. The term cycloalkyl preferably means C₃-C₁₂ cycloalkyl and includes monocyclic, bicyclic and polycyclic radicals. Alkyl, alkenyl, aryl, heteroaryl and cycloalkyl radicals may be substituted or unsubstituted. Preferred substituents are as indicated above.

The compounds of the present invention and complexes thereof with suitable chelants, e.g. a NMR-active or radioactive moiety, such as a metal atom or ion, exhibit a regulated and controlled biodistribution. Thus, they are suitable for the manufacture of pharmaceutical compositions for diagnosis or therapy, e.g. NMR diagnosis, radiodiagnosis or radiotherapy.

### Experimental Details

### Detailed Description of the Invention

The invention is illustrated further by reference to the following non-limiting examples.

Products were characterized and identified by NMR (¹H NMR, ¹³C NMR, ³¹P NMR and IR), MS spectroscopy, elementar analysis and volumetric or HPLC analysis in some cases.

### Example 1

### Preparation of methyl (2R)-2-{[(1S)-2-ethoxy-1-methyl-2-oxoethyl]amino}-3-(4-nitrophenyl)propanoate (A-Ia) and methyl (2R)-2-{[(1R)-2-ethoxy-1-methyl-2-oxoethyl]amino}-3-(4-nitrophenyl)propanoate (A-Ib)

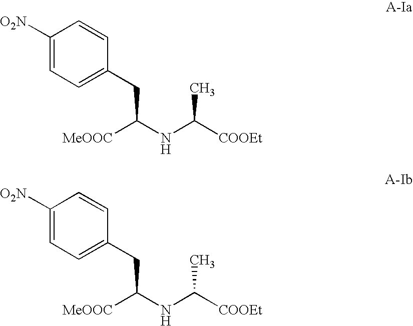

Method A

p-nitro-D-phenylalanine methyl ester hydrochloride (1.3 g; 5 mmol) and ethyl 2-bromopropionate (7.24 g; 40 mmol) were dissolved in 10 ml of dry DMF. NaH (120 mg; 5 mmol), Et₃N (4 g; 40 mmol) and Kl (6.64 g, 40 mmol) were added to the solution and the mixture was stirred at 80-100° C. for 24 h. Then H₂O was added to quench the reaction and the pH was adjusted to 8-9. After concentrating the DMF-water solution by vacuum destillation, the residual oil was extracted with CHCl₃. The CHCl₃ layer was dried (Na₂SO₄) and concentrated. The residue was purified by column chromatography on silica gel.

### Example 2

### Preparation of methyl (2R)-2-{[(1S)-2-ethoxy-1-methyl-2-oxoethyl]amino}-3-(4-nitrophenyl)propanoate (A-Ia) and methyl (2R)-2-{[(1R)-2-ethoxy-1-methyl-2-oxoethyl]amino}-3-(4-nitrophenyl)propanoate (A-Ib)

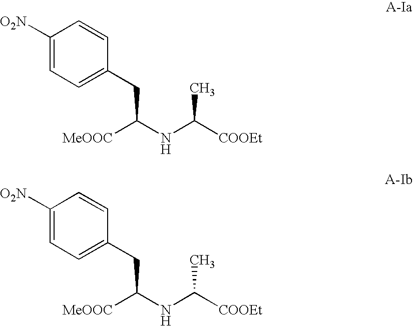

Method B

p-nitro-D-phenylalanine methyl ester hydrochloride (1.3 g; 5 mmol) and ethyl 2-bromopropionate (7.24 g; 40 mmol) were dissolved in 10 ml of dry DMF. Pyridine (3.16 g; 40 mmol) and silver oxide (4.63 g; 20 mmol) were added to the solution and the mixture was stirred at room temperature for 5 h. The precipitate was filtered. H₂O (10 ml) was added to quench the reaction and the pH was adjusted to 8-9. After concentrating the DMF-water solution by vacuum destillation, the residual oil was extracted with CHCl₃. The CHCl₃ layer was dried (Na₂SO₄) and concentrated and the residue was purified by column chromatography on silica gel.

### Example 3

### Preparation of methyl (2R)-2-{[(1S)-2-ethoxy-1-methyl-2-oxoethyl]amino}-3-(4-nitrophenyl)propanoate (A-Ia) and methyl (2R)-2-{[(1R)-2-ethoxy-1-methyl-2-oxoethyl]amino}-3-(4-nitrophenyl)propanoate (A-Ib)

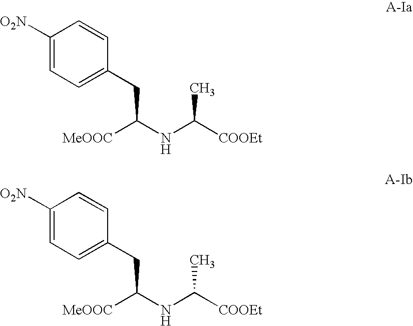

Method C

To a suspension of p-nitro-D-phenylalanine methyl ester hydrochloride (2.6 g; 10 mmol) and NaH (240 mg; 10 mmol) in 15 ml anhydrous methanol was added pyruvic acid ethyl ester (1.28 g; 11 mmol) at room temperature, and the mixture was stirred for 1 h. The reaction mixture was then stirred with Na [BH₃(CN)] (0.63 g; 10 mmol) at room temperature for 22 h. The white precipitate formed was filtered (S4) and washed with methanol and ether. The filtrate and washings were combined and concentrated in vacuo to give a crude mixture of a monoalkylated product, the starting material and a by-product, which were separated by column chromatography on Sephadex LH-20.

### Example 4

### Preparation of methyl (2R)-2{[(1S)-2-ethoxy-1-methyl-2-oxoethyl]amino}-3-(4-nitrophenyl)propanoate (A-Ia) and methyl (2R)-2-{[(1R)-2-ethoxy-1-methyl-2-oxoethyl]amino}-3-(4-nitrophenyl)propanoate (A-Ib)

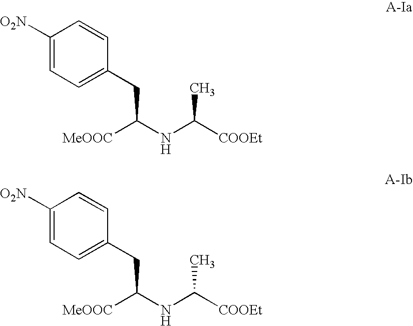

Method D

A) p-nitro-D-phenylalanine methyl ester hydrochloride (0.26 g; 1 mmol) and (R)-ethyl 2-iodopropionate (2 g; 9 mmol) were dissolved in 10 ml of dry DMF. Triethylamine (1.01 g; 10 mmol) was added to the solution, and the mixture was stirred at 80-100° C. for 48 h. Purification of the reaction mixture by a similar procedure to that described in example 1 method A. An enantiomer pure product A-Ia is obtained.

B) Compounds A-Ib was prepared from p-nitro-D-phenylalanine methyl ester hydrochloride by the same method as it has been described in this Example with ethyl (2S)-2-[(methylsulfonyl)oxy]propanoate on place of (R)-ethyl 2-iodopropionate.

### Example 4

### Preparation of methyl (2R)-2-{[(1S)-2-ethoxy-1-methyl-2-oxoethyl]amino}-3-(4-nitrophenyl)propanoate (A-Ia) and methyl (2R)-2-{[(1R)-2-ethoxy-1-methyl-2-oxoethyl]amino}-3-(4-nitrophenyl)propanoate (A-Ib)

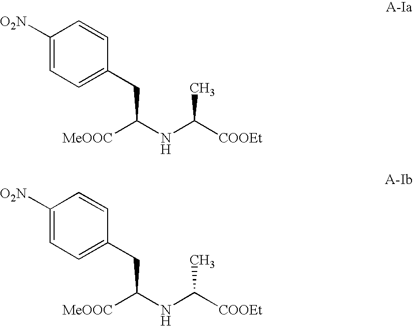

Method E

A dry K₂CO₃ (2.07 g; 15 mmol) was added to an acetonitrile solution of N-nosyl-p-nitro-D-phenylalanine methyl ester (4.09 g; 10 mmol) and TEBA (228 mg; 1 mmol) in argon inert atmosphere. The heterogeneous mixture was stirred at 55° C. and then ethyl 2-bromopropionate was added dropwise. The reaction mixture was warmed and stirred until no starting material was detectable with TLC analysis. Cooled to room temperature, diluted with water (50 ml) and extracted with dichloromethane. The organic layer was washed with water and brine, dried over Na₂SO₄ and evaporated under vacuum to give pure N-alkyl sulfonamide. Then it was added anhydrous potassium carbonate (45 mmol) to a solution of the N-alkyl sulfonamide and thiophenol (3.85 g; 35 mmol) in acetonitrile*. The reaction mixture was stirred at room temperature overnight or stirred at 50° C. for 1 h. The resulting solution was reduced under vacuum and the residue taken up in diethyl ether. After usual workup the crude following esters were isolated. Esters were separated by silicagel column chromatography.

*Alternative method for deprotection of nosyl group was used lithium hydroxide and thioglycolic acid in DMF at 25° C.

### Example 4

### Preparation of methyl (2R)-2-{[(1S)-2-ethoxy-1-methyl-2-oxoethyl]amino}-3-(4-nitrophenyl)propanoate (A-Ia) and methyl (2R)-2-{[(1R)-2-ethoxy-1-methyl-2-oxoethyl]amino}-3-(4-nitrophenyl)propanoate (A-Ib)

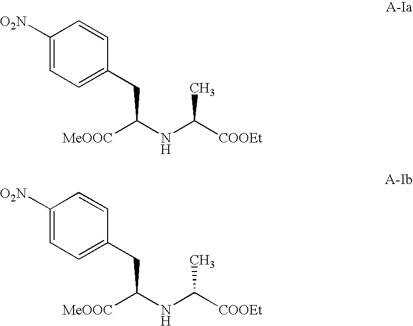

Method F

A) A solution of methyl (2S)-2-hydroxy-3-(4-nitrophenyl)propanoate (3.6 g; 16 mmol) and pyridine (1.26 g; 16 mmol) in 15 ml dichloromethane was added dropwise over 20 min at −5° C. to a solution of trifluoromethane sulfonic anhydride (4.51 g; 16 mmol) in 15 ml dichloromethane. After returning to room temperature, the mixture was concentrated. Pentane (50 ml) was added and the solid form was removed by filtration. The filtrate was concentrated. The oily residue was dissolved in 30 ml dichloromethane and added dropwise at −70° C. over 1 h to a solution of ethyl (2S)-2-aminopropanoate (3.75 g; 32 mmol) and triethylamine (1.62 g; 16 mmol) in 30 ml dichloromethane. The mixture was stirred for 1 h at −70° C. and then for 16 h at room temperature. The solid form was removed by filtration. The residue obtained by concentration of the filtrate was purified by column chromatography. It is obtained an enantiomer pure product A-Ia.

B) Compound A-Ib was prepared from methyl (2S)-2-hydroxy-3-(4-nitrophenyl)propanoate by the same method as it has been described in this Example with ethyl (2R)-2-aminopropanoate on place of ethyl (2S)-2-aminopropanoate.

### Example 5

### Preparation of methyl (2R)-2-{[(1S)-2-ethoxy-1-methyl-2-oxoethyl]amino}-3-(4-nitrophenyl)propanoate (A-Ia) and methyl (2R)-2-{[(1R)-2-ethoxy-1-methyl-2-oxoethyl]amino}-3-(4-nitrophenyl)propanoate (A-Ib)

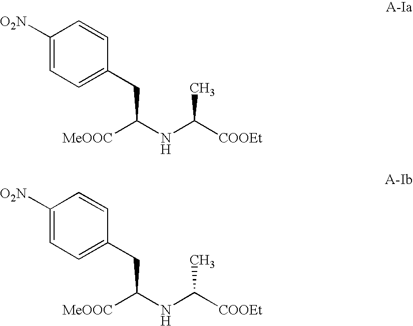

Method G

A) A solution of DEAD (3.48 g; 20 mmol) in benzene (8 ml) is added dropwise to a solution of ethyl (2R)-2-hydroxypropanoate (2.36 g; 20 mmol), p-nitro-D-phenylalanine methyl ester (2.6 g; 10 mmol) and triphenylphosphine (5.25 g; 20 mmol) in tetrahydrofuran (20 ml) at room temperature. After the solution has been stirred for 2 h at room temperature, the solvent is removed in vacuo. Ether is added to the residue to precipitate triphenylphosphine oxide and diethyl hydrazinedicarboxylate which are filtered off. The filtrate is evaporated and the residue is applied to a silica gel column which is eluted. It is obtained an enantiomer pure product A-Ia.

B) Compound A-Ib was prepared from p-nitro-D-phenylalanine methyl ester by the same method as it has been described in this Example with ethyl (2S)-2-hydroxypropanoate on place of ethyl (2R)-2-hydroxypropanoate.

### Example 6

### Preparation of methyl (2S)-2-{[(1S)-2-ethoxy-1-methyl-2-oxoethyl]amino}-3-(4-nitrophenyl)propanoate (A-Ic) and methyl (2S)-2-{[(1R)-2-ethoxy-1-methyl-2-oxoethyl]amino}-3-(4-nitrophenyl)propanoate (A-Id)

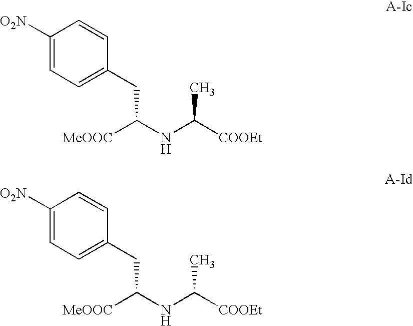

Method A

Compounds A-Ic and A-Id were prepared by the same method as it has been described in example 1 method A with p-nitro-L-phenylalanine methyl ester hydrochloride on place of p-nitro-D-phenylalanine methyl ester hydrochloride.

### Example 7

### Preparation of methyl (2S)-2-{[(1S)-2-ethoxy-1-methyl-2-oxoethyl]amino}-3-(4-nitrophenyl)propanoate (A-Ic) and methyl (2S)-2-{[(1R)-2-ethoxy-1-methyl-2-oxoethyl]amino}-3-(4-nitrophenyl)propanoate (A-Id)

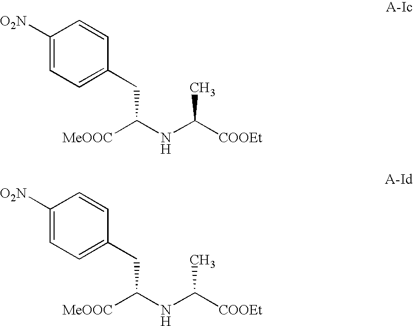

Method B

p-nitro-L-phenylalanine methyl ester hydrochloride (1.3 g; 5 mmol) and ethyl 2-bromopropionate (7.2 g; 40 mmol) and sodium iodide (6.74 g; 45 mmol) were dissolved in 10 ml of dry DMF. Pyridine (3.16 g; 40 mmol) and silver oxide (4.63 g; 20 mmol) were added to the solution, and the mixture was stirred at room temperature for 5 h. The precipitate was filtered. H₂O (10 ml) was added to quench the reaction, and the pH was adjusted to 8-9. After concentrating the DMF-water solution by vacuum destillation, the residual oil was extracted with CHCl₃. The CHCl₃ layer was dried (Na₂SO₄) and concentrated, and the residue was purified by column chromatography on silica gel.

### Example 8

### Preparation of methyl (2S)-2-{[(1S)-2-ethoxy-1-methyl-2-oxoethyl]amino}-3-(4-nitrophenyl)propanoate (A-Ic) and methyl (2S)-2-{[(1R)-2-ethoxy-1-methyl-2-oxoethyl]amino}-3-(4-nitrophenyl)propanoate (A-Id)

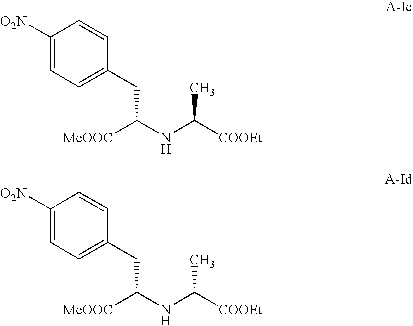

Method C

Pyruvic acid ethyl ester (1.28 g; 11 mmol) was added at room temperature to a suspension of p-nitro-L-phenylalanine methyl ester hydrochloride (2.6 g; 10 mmol) and anhydrous NaOAc (3.28 g; 40 mmol) in 15 ml absolute methanol and the mixture was stirred for 1 h. The reaction mixture was then stirred with Na[BH₃(CN)] (0.63 g; 10 mmol) at room temperature for 22 h. The white precipitate form was filtered (S4) and washed with methanol and ether. The filtrate and washings were combined and concentrated in vacuo to give a crude mixture of a monoalkylated product, the starting material and a by-product, which were separated by column chromatography on silica gel.

### Example 9

### Preparation of methyl (2S)-2-{[(1S)-2-ethoxy-1-methyl-2-oxoethyl]amino}-3-(4-nitrophenyl)propanoate (A-Ic) and methyl (2S)-2-{[(1R)-2-ethoxy-1-methyl-2-oxoethyl]amino}-3-(4-nitrophenyl)propanoate (A-Id)

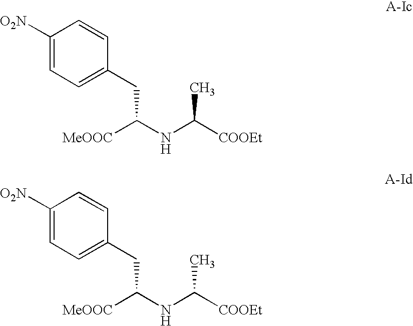

Method D

A) p-nitro-L-phenylalanine methyl ester hydrochloride (260 mg; 1 mmol) and (R)-ethyl 2-iodopropionate (9 mmol) were dissolved in 10 ml of dry DMF. Triethylamine (10 mmol) was added to the solution, and the mixture was stirred at 80-100° C. for 48 h. Purification of the reaction mixture by a similar procedure to that described in example 1 method A. An enantiomer pure product A-Ic is obtained.

B) Compound A-Id was prepared from p-nitro-D-phenylalanine methyl ester hydrochloride by the same method as it has been described in this Example with ethyl (2S)-2-[(methylsulfonyl)oxy]propanoate on place of (S)-ethyl 2-iodopropionate. An enantiomer pure product A-Id is obtained.

C) p-nitro-L-phenylalanine methyl ester hydrochloride (260 mg; 1 mmol) and (R)-ethyl 2-(nosyloxy)propionate (3.64 g; 1.2 mmol) were dissolved in 10 ml of dry DMF. Triethylamine (1.01 g; 10 mmol) was added to the solution, and the mixture was stirred at 80-100° C. for 48 h. Purification of the reaction mixture by a similar procedure to that described in example 1 method A. An enantiomer pure product A-Ic is obtained.

D) Compound A-Id was prepared from p-nitro-D-phenylalanine methyl ester hydrochloride by the same method as it has been described in this Example point B) with ethyl (2S)-2-[(p-tolylsulfonyl)oxy]propanoate on place of ethyl (2S)-2-[(methylsulfonyl)oxy]propanoate. An enantiomer pure product A-Id is obtained.

### Example 10

### Preparation of methyl (2S)-2-{[(1S)-2-ethoxy-1-methyl-2-oxoethyl]amino}-3-(4-nitrophenyl)propanoate (A-Ic) and methyl (2S)-2-{[(1R)-2-ethoxy-1-methyl-2-oxoethyl]amino}-3-(4-nitrophenyl)propanoate (A-Id)

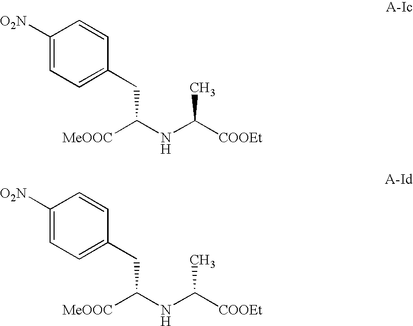

Method E

A dry K₂CO₃ (2 g; 15 mmol) was added to an acetonitrile solution of N-nosyl-p-nitro-L-phenylalanine methyl ester* (4.1 g; 10 mmol) and triethylbenzylammonium chloride (0.23 g; 1 mmol) in argon inert atmosphere. The heterogeneous mixture was stirred at 55° C. and then ethyl 2-bromopropionate (3.62 g; 20 mmol) was added dropwise. Reaction mixture was warmed and stirred until no starting material was detectable with TLC analysis. Cooled to room temperature, diluted with water (50 ml) and extracted with dichloromethane. The organic layer was washed with water and brine, dried over Na₂SO₄ and evaporated under vacuum to give pure N-alkyl sulfonamide. Then it was added anhydrous potassium carbonate (6.2 g; 45 mmol) to a solution of the N-alkyl sulfonamide and thiophenol (3.85 g; 35 mmol) in acetonitrile**. The reaction mixture was stirred at room temperature overnight or stirred at 50° C. for 1 h. The resulting solution was reduced under vacuum and the residue taken up in diethyl ether. After usual workup the crude following esters were isolated. Esters were separated by silicagel column chromatography.

* N-nosyl-p-nitro-L-phenylalanine methyl ester was prepared by reaction equimolar amount of p-nitro-L-phenylalanine methyl ester hydrochloride with nitrophenylsulfonyl chloride in anhydrous dichloromethane at 0° C. and anhydrous triethylamine. Stirring was continued at 25° C. until no starting material was detectable (TLC). The reaction mixture was washed with water and the organic phase was dried over sodium sulfate, evaporated to dryness under vacuum and purified by flash column chromatography on silica gel.

** Alternative method for deprotection of nosyl group was used lithium hydroxide and thioglycolic acid in DMF at 25° C.

### Example 11

### Preparation of methyl (2S)-2-{[(1S)-2-ethoxy-1-methyl-2-oxoethyl]amino}-3-(4-nitrophenyl)propanoate (A-Ic) and methyl (2S)-2-{[(1R)-2-ethoxy-1-methyl-2-oxoethyl]amino}-3-(4-nitrophenyl)propanoate (A-Id)

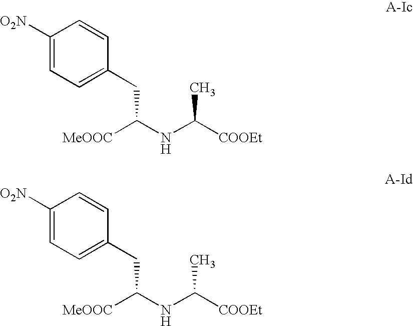

Method F

A) A solution of methyl (2R)-2-hydroxy-3-(4-nitrophenyl)propanoate (7.2 g; 32 mmol) and pyridine (2.77 g; 35 mmol) in 30 ml dichloromethane was added dropwise over 20 min at −5° C. to a solution of trifluoromethane sulfonic anhydride (9 g; 32 mmol) in 40 ml dichloromethane. After returning to room temperature, the mixture was concentrated. Pentane (120 ml) was added and the solid form was removed by filtration. The filtrate was concentrated. The oily residue was dissolved in 60 ml dichloromethane and added dropwise at −70° C. over 1 h to a solution of ethyl (2S)-2-aminopropanoate (7.61 g; 65 mmol) and triethylamine (3.23 g; 32 mmol) in 60 ml dichloromethane. The mixture was stirred for 1 h at −70° C. and then for 16 h at room temperature. The solid form was removed by filtration. The residue obtained by concentration of the filtrate was purified by column chromatography. An enantiomer pure product A-Ic is obtained.

B) Compound A-Id was prepared from methyl (2R)-2-hydroxy-3-(4-nitrophenyl)propanoate by the same method as it has been described in this Example with ethyl (2R)-2-aminopropanoate on place of ethyl (2S)-2-aminopropanoate.

### Example 12

### Preparation of methyl (2S)-2-{[(1S)-2-ethoxy-1-methyl-2-oxoethyl]amino}-3-(4-nitrophenyl)propanoate (A-Ic) and methyl (2S)-2-{[(1R)-2-ethoxy-1-methyl-2-oxoethyl]amino}-3-(4-nitrophenyl)propanoate (A-Id)

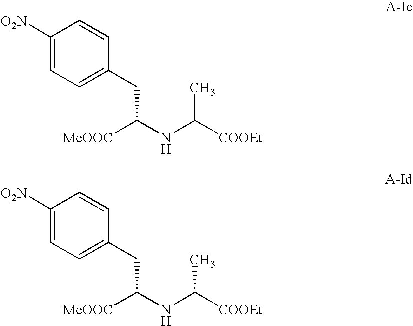

Method G

A) A solution of DEAD (3.48 g; 20 mmol) in benzene (8 ml) is added dropwise to a solution of ethyl (2R)-2-hydroxypropanoate (2.36 g; 20 mmol), p-nitro-L-phenylalanine methyl ester (2.6 g; 10 mmol) and triphenylphosphine (5.25 g; 20 mmol) in tetrahydrofuran (20 ml) at room temperature. After the solution has been stirred 2 h at room temperature, the solvent is removed in vacuo. Ether is added to the residue to precipitate triphenylphosphine oxide and diethyl hydrazinedicarboxylate which are filtered off. The filtrate is evaporated and the residue is applied to a silica gel column which is eluted. An enantiomer pure product A-Ic is obtained.

B) Compound A-Id was prepared from p-nitro-L-phenylalanine methyl ester by the same method as it has been described in this Example with ethyl (2S)-2-hydroxypropanoate on place of ethyl (2R)-2-hydroxypropanoate.

C) A solution of DEAD (3.48 g; 20 mmol) in dichloromethane (8 ml) is added dropwise to a solution of ethyl (2R)-2-hydroxypropanoate (2.36 g; 20 mmol), N-(2,4-dinitrophenylsulfonyl)-p-nitro-L-phenylalanine methyl ester* (4.54 g; 10 mmol) or N-nosyl-p-nitro-L-phenylalanine methyl ester (4.09 g; 10 mmol) and triphenylphosphine (5.25 g; 20 mmol) in 20 ml dichloromethane at room temperature. After the solution has been stirred 30 min at room temperature, the solvent is removed in vacuo. Ether is added to the residue to precipitate triphenylphosphine oxide and diethyl hydrazinedicarboxylate which are filtered off. The filtrate is evaporated. Then it was added anhydrous potassium carbonate (6.2 g; 45 mmol) to a solution of the N-alkyl sulfonamide and thiophenol (3.9 g; 35 mmol) in acetonitrile**. The reaction mixture was stirred at room temperature overnight or stirred at 50° C. for 1 h. The resulting solution was reduced under vacuum and the residue taken up in diethyl ether. After usual workup the crude following esters were isolated. Esters were separated by silicagel column chromatography. An enantiomer pure product A-Ic is obtained.

* N-(2,4-dinitrophenylsulfonyl)-p-nitro-L-phenylalanine or N-nosyl-p-nitro-L-phenylalanine methyl ester was prepared by reaction of p-nitro-L-phenylalanine methyl ester hydrochloride (1 eq) with 2,4-dinitrophenylsulfonyl chloride (1 eq) or nitrophenylsulfonyl chloride (1 eq) in anhydrous dichloromethane at 0° C. and anhydrous triethylamine. Stirring was continued at 25° C. until no starting material was detectable (TLC). The reaction mixture was washed with water and the organic phase was dried over sodium sulfate, evaporated to dryness under vacuum and purified by flash column chromatography on silica gel.

** Alternative method for deprotection of nosyl group was used lithium hydroxide and thioglycolic acid in DMF at 25° C.

### Example 13

### Preparation of alkyl (2R/S)-2-{[(1R/S)-2-ethoxy-1-R₃-2-oxoethyl]amino}-3-(4-nitrophenyl)pronanoats (A-IIa-d-A-XXIa-d)

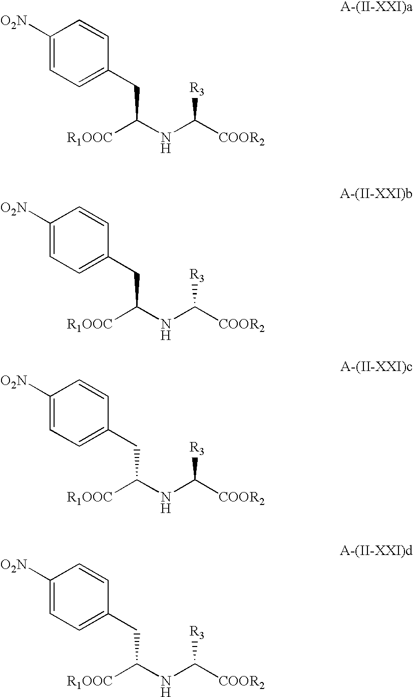

Table 1 summarizes the results of diesters A-(I-XXI)a-d preparations by methods A, B, C, D, E, F, G.

TABLE 1

| | Substance a | Substance b | Substance c | Substance d |
|---|---|---|---|---|
| | Method/ | Method/ | Method/ | Method/ |
| Carbon-backbone | Yield (%) | Yield (%) | Yield (%) | Yield (%) |
| A-II (R₁ = Me, R₂ = Me, R₃ = Me) | G/92 | E/73 | F/90 | C/38 |
| A-III (R₁ = Et, R₂ = Et, R₃ = Me) | A/51 | A/57 | G/86 | F/81 |
| A-IV (R₁ = Et, R₂ = Me, R₃ = Me) | A/47 | A/50 | G/85 | G/78 |
| A-V (R₁ = Me, R₂ = Et, R₃ = Et) | F/82 | F/85 | A/49 | C/45 |
| A-VI (R₁ = Me, R₂ = Me, R₃ = Pr) | D/41 | B/36 | G/85 | G/91 |
| A-VII (R₁ = Me, R₂ = Me, R₃ = Bu) | A/55 | A/47 | E/72 | E/76 |
| A-VIII (R₁ = Me, R₂ = Me, | F/76 | F/80 | A/53 | A/55 |
| R₃ = hexyl) | | | | |
| A-IX (R₁ = Me, R₂ = Me, R₃ = octyl) | F/73 | F/76 | A/43 | A/40 |
| A-X (R₁ = Me, R₂ = Me, R₃ = decyl) | A/71 | A/48 | A/48 | A/52 |
| A-XI (R₁ = Me, R₂ = Me, | F/68 | F/73 | C/28 | C/32 |
| R₃ = tetradecyl) | | | | |
| A-XII (R₁ = Me, R₂ = Me, | F/65 | F/69 | E/74 | E/76 |
| R₃ = hexadecyl) | | | | |
| A-XIII (R₁ = Me, R₂ = Me, | E/75 | E/79 | E/82 | E/76 |
| R₃ = cyclohexylmethyl) | | | | |
| A-XIV (R₁ = Me, R₂ = Me, R₃ = p- | A/56 | F/82 | D/22 | — |
| nitrobenzyl) | | | | |
| A-XV (R₁ = Me, R₂ = Me, R₃ = p- | E/62 | E/59 | E/60 | E/64 |
| methoxybenzyl) | | | | |
| A-XVI (R₁ = Me, R₂ = Me, | G/93 | G/90 | A/32 | A/35 |
| R₃ = benzyl) | | | | |
| A-XVI (R₁ = Me, R₂ = Me, R₃ = p- | D/22 | D/18 | E/72 | E/74 |
| iodobenzyl) | | | | |
| A-XVII (R₁ = Me, R₂ = Me, | A/55 | A/50 | A/47 | A/49 |
| R₃ = CH₂OBz) | | | | |
| A-XVIII (R₁ = Me, R₂ = Me, | E/75 | E/72 | G/74 | G/76 |
| R₃ = CH₂SBz) | | | | |
| A-XIX (R₁ = Me, R₂ = Me, | F/71 | F/68 | A/42 | C/25 |
| R₃ = CH₂COOH) | | | | |
| A-XX (R₁ = Me, R₂ = Me, | F/73 | F/76 | A/51 | C/22 |
| R₃ = CH₂CH₂COOH) | | | | |
| A-XXI | E/82 | A/31 | E/73 | G/78 |
| (R₁ = Me, R₂ = Me, | | | | |
| R₃ = CH₂CH₂CH₂CH₂NHBoc) | | | | |

### Example 14

### General Procedure for Preparation of (2R/S)-2-{[(1RS)-2-amino-1-R₃-2-oxoethyl]amino}-3-(4-nitrophenyl)propanamide (B-Ia-d-B-XXIa-d)

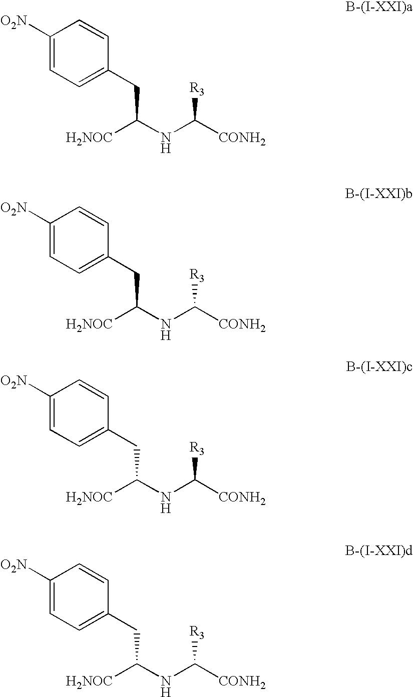

An oil of diester A-IIa (12.41 g; 40 mmol) was added to dry 250 ml methanol previously saturated with ammonia gas at −16° C. tightly stoppered and left at −16° C. for 14 days. After this time is the transformation quantitative (TLC analysis). The solution is carefully warmed up to laboratory temperature. A most of sorbed ammonia is forced out by the stream of nitrogen. Then the solution is evaporated at RWO under reduced pressure. It is obtained clear diamid B-IIa.

Table 2 summarizes the results of diamide B-(I-XXI)a-d preparations from diesters A-(I-XXI)a-d.

TABLE 2

| | Substance a | Substance b | Substance c | Substance d |
|---|---|---|---|---|
| Carbon-backbone | Yield (%) | Yield (%) | Yield (%) | Yield (%) |
| B-I (R₃ = Me) | 100 | 98 | 100 | 99 |
| B-V (R₃ = Et) | 99 | 100 | 98 | 99 |
| B-VI (R₃ = Pr) | 98 | 99 | 99 | 100 |
| B-VII (R₃ = Bu) | 100 | 99 | 100 | 99 |
| B-VIII (R₃ = hexyl) | 100 | 100 | 98 | 98 |
| B-IX (R₃ = octyl) | 98 | 99 | 99 | 100 |
| B-X (R₃ = decyl) | 99 | 100 | 98 | 100 |
| B-XI (R₃ = tetradecyl) | 100 | 100 | 100 | 100 |
| B-XII (R₃ = hexadecyl) | 99 | 100 | 99 | 100 |
| B-XIII | 98 | 99 | 100 | 99 |
| (R₃ = cyclohexylmethyl) | | | | |
| B-XIV (R₃ = p-nitrobenzyl) | 100 | 98 | 100 | — |
| B-XV (R₃ = p- | 99 | 100 | 99 | 98 |
| methoxybenzyl) | | | | |
| B-XVI (R₃ = benzyl) | 100 | 99 | 97 | 100 |
| B-XVI (R₃ = p-iodobenzyl) | 98 | 100 | 100 | 100 |
| B-XVII (R₃ = CH₂OBz) | 97 | 98 | 99 | 98 |
| B-XVIII (R₃ = CH₂SBz) | 99 | 100 | 100 | 99 |
| B-XIX (R₃ = CH₂COOH) | 100 | 99 | 98 | 100 |
| B-XX | 97 | 97 | 99 | 98 |
| (R₃ = CH₂CH₂COOH) | | | | |
| B-XXI | 99 | 100 | 100 | 99 |
| (R₃ = CH₂CH₂CH₂CH₂NHBoc) | | | | |

### Example 15

### Generals Procedures for Preparation of N-[(1R/S)-2-amino-1-R₃-ethyl]-N-[(1R/S)-2-amino-1-(4-nitrobenzyl)ethyl]amine (C-Ia-d-C-XXIa-d)

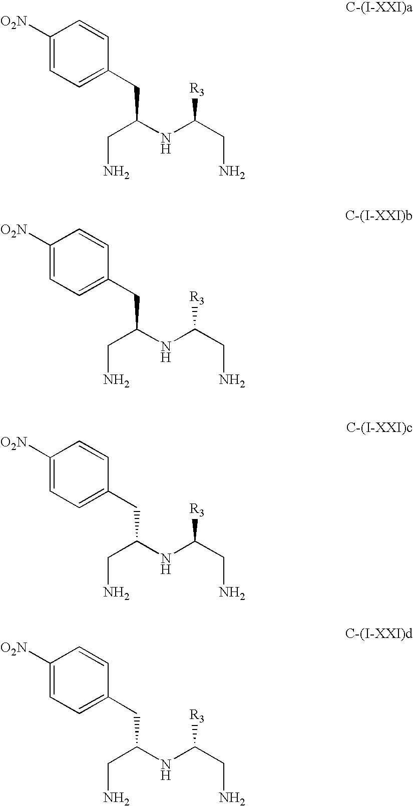

Method A (BH₃.THF)

The diamid B-Ia (19.61 g; 70 mmol) was suspended in 100 ml of dry THF. 840 ml of a 1 M borane solution were added drop-wise at 0° C. The mixture was stirred during 1 h at 5° C. under inert atmosphere and was then left at room temperature. The solution was heated for 12 h at 25° C. and then cooled at 5° C. 50 ml of dry methanol was added slowly to destroy the borane excess. The solution was evaporated under reduced pressure and the residue was again treated with 80 ml of methanol. The solvent was evaporated and the residue was diluted in 350 ml of 4 M aqueous solution of hydrochloric acid and refluxed over 12 h. The solution was evaporated, the residue dissolved in dematerialized H₂O, and the pH of mixture adjusted with concentrated NH₄OH and 5 M KOH to 11. The aqueous solution was extracted with ten 150 ml portions of CHCl₃. The organic layer was separated, washed with brine, dried over anhydrous Na₂SO₄, and the solvent was removed under reduced pressure. Purification of the residue by silica gel chromatography gave pure amine C-Ia in the indicated yields.

Method B (NaBH₄+BF₃.Et₂O)

A solution of boron trifluoride etherate (852 mg; 6 mmol) in tetrahydrofurane (10 ml) was added slowly to a room temperature solution of NaBH₄ (227 mg; 6 mmol) and amide B-Ib (280 mg; 1 mmol) in tetrahydrofurane (25 ml) under an inert atmosphere. The mixture was heated to reflux until TLC monitoring showed complete consumption of the substrate. The reaction mixture was cooled to 0° C., quenched with water (caution: vigorous gas evolution) keeping the temperature ≦15° C. After 30 min, the THF was removed under reduced pressure. The residue dissolved in EtOH (10 ml) and 6M HCl (10 ml), and the resulting solution refluxed for 18 h. The solution was evaporated, the residue dissolved in dematerialized H₂O, and the pH of mixture adjusted with concentrated NH₄OH and 5 M KOH to 11.5±0.5. The aqueous solution was extracted with ten 10 ml portions of CHCl₃. The organic layer was separated, washed with brine, dried over anhydrous Na₂SO₄, and the solvent was removed under reduced pressure. Purification of the residue by silica gel chromatography gave pure amine C-Ib in the indicated yields.

Method C (NaBH₄+CH₃SiCl) or (LiBH₄+CH₃SiCl)*

NaBH₄ (3003 mg; 8 mmol) was added to a solution of Me₃SiCl (174 mg; 1.6 mmol) in THF (8 ml) and the mixture refluxed for 2 h under nitrogen atmosphere. A solution of amide B-Ic (280 mg; 1 mmol) in THF (10 ml) was then added dropwise over the course of 5 min. The solution was refluxed for a further 15 h. After cooling, 10 ml MeOH were cautiously added and the volatiles removed in vacuo. The residue dissolved in 6M HCl (10 ml), and the resulting solution refluxed for 16 h. The solution was evaporated, the residue dissolved in 10 ml of water, and the pH was adjusted to 14 (pH paper) with 50% aqueous sodium hydroxide. The aqueous solution was extracted with six 15 ml portions of dichloromethane, and the dichloromethane extracts were combined and dried anhydrous Na₂SO₄. Filtration and evaporation of the solvent at reduced pressure on a rotary evaporator gave an amber oil, which was purified by flash chromatography on silica gel using chloroform/methanol/concentrated aqueous ammonium hydroxide as the eluant to provide pure amine C-Ic.

*Lithium Borohydride Procedure

This procedure is identical to the NaBH₄+CH₃SiCl procedure with the exception of the substitution of LiBH₄ for NaBH₄ on a molar basis and the fact that the mixture of LiBH₄+CH₃SiCl is not warmed up for 2 h in advance.

Method D (NaBH₄+I₂/THF)

In three neck round-bottom flask equipped with a magnetic stirbar, reflux condenser, thermometer, and addition funnel was flushed with argon and charged with 10 ml of THF and 294 mg of amide B-Vd (1 mmol), and 302 mg of NaBH₄ (8 mmol) Then, a solution of 381 mg I₂ (1.5 mmol) in 10 ml of THF was added slowly and dropwise at the temperature of 25-40° C. After the addition was complete, the flask was heated to reflux overnight. Excess reducing agent was cautiously destroyed by dropwise addition of 5 ml of methanol at 10° C. The solvents were then removed in vacuo, and the residue was taken up in 100 ml of 20% aqueous KOH and the product extracted 7× with 50 ml of dichloromethane. After drying (Na₂SO₄), the extract was evaporated to an oil amine C-Vd.

Table 3 summarizes the results of diamide B-(I-XXI)a-d reductions to triamine C-(I-XXI)a-d.

TABLE 3

| | Substance a | Substance b | Substance c | Substance d |
|---|---|---|---|---|
| | Method/ | Method/ | Method/ | Method/ |
| Carbon-backbone | Yield (%) | Yield (%) | Yield (%) | Yield (%) |
| C-I (R₃ = Me) | A/78 | B/80 | C/76 | C/82 |
| C-V (R₃ = Et) | B/68 | A/85 | B/72 | D/52 |
| C-VI (R₃ = Pr) | C/85 | C/72 | C/70 | C/75 |
| C-VII (R₃ = Bu) | A/79 | B/77 | B/84 | B/71 |
| C-VIII (R₃ = hexyl) | B/83 | A/69 | C/79 | B/85 |
| C-IX (R₃ = octyl) | D/75 | C/79 | A/84 | B/82 |
| C-X (R₃ = decyl) | B/89 | A/86 | C/85 | C/80 |
| C-XI (R₃ = tetradecyl) | A/90 | C/83 | B/72 | C/87 |
| C-XII (R₃ = hexadecyl) | C/95 | C/93 | C/90 | C/91 |
| C-XIII | C/92 | D/71 | C/88 | A/90 |
| (R₃ = cyclohexylmethyl) | | | | |
| C-XIV (R₃ = p-nitrobenzyl) | C/79 | C/82 | C/76 | — |
| C-XV (R₃ = p- | A/73 | B/78 | A/79 | A/83 |
| methoxybenzyl) | | | | |
| C-XVI (R₃ = benzyl) | A/86 | B/83 | C/80 | A/82 |
| C-XVI (R₃ = p-iodobenzyl) | A/85 | A/83 | D/71 | A/80 |
| C-XVII (R₃ = CH₂OBz) | C/62 | C/69 | C/72 | C/65 |
| C-XVIII (R₃ = CH₂SBz) | A/54 | A/60 | A/64 | D/63 |
| C-XIX (R₃ = CH₂CH₂OH) | A/48 | C/67 | A/58 | B/67 |
| C-XX | C/62 | C/65 | C/58 | B/54 |
| (R₃ = CH₂CH₂CH₂OH) | | | | |
| C-XXI | A/79 | A/85 | C/90 | C/80 |
| (R₃ = CH₂CH₂CH₂CH₂NHBoc) | | | | |

### Example 16

### Procedures for Preparation (D-Ia-d-D-XXIa-d)

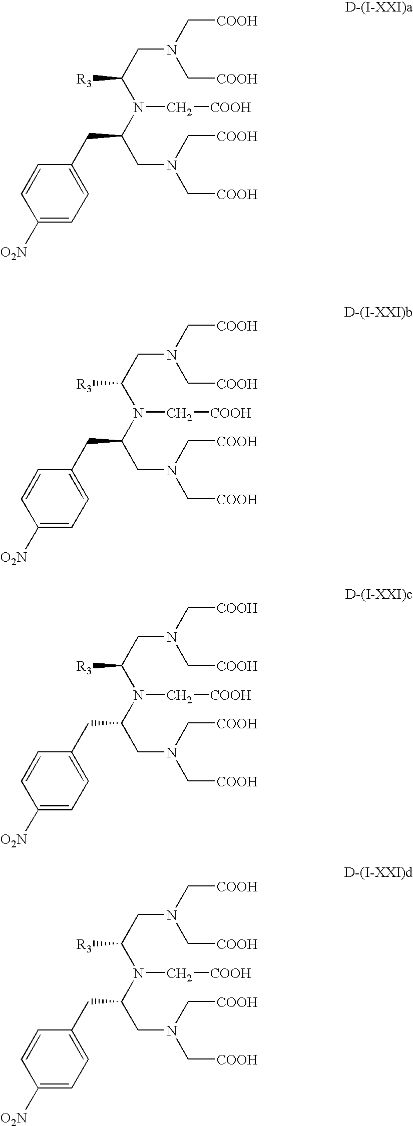

Method A: Tert.-Butyl Bromoacetate and N-methyl-N,N-diisopropylamine in DMF

(29 g; 115 mmol) of C-I in 1600 ml of dried dimethylformamide (DMF) were placed into a 5 l three necked reaction vessel equipped with an addition funnel (with servo and pressure correction), electronic temperature meter bonded to thermostat, nitrogen overpressure inlet adapter and stirring apparatus. (97.9 g; 0.85 mol) of N-methyl-N,N-diisopropylamine (of a purity better than 98%) in 300 ml of dried DMF were added thereafter. (156 g; 0.8 mol) of tert.-butyl bromoacetate in 1000 ml of dried dimethylformamide were added to the solution at 25° C. over the period of 60 minutes. After addition, the temperature was slowly raised up to 65° C. and this mixture has been stirred at 65° C. under nitrogen overpressure for 24 hours. Thereafter, reaction mass was poured into 7 liters of 15° C. water with vigorous stirring. A separated oily product was extracted by 4×300 ml of dichloromethane. After evaporation is the product chromatographed on Silica (tert.-Butanol-dichloromethane, 2:3 mixture). After evaporation of appropriate fractions, a brownish yellow oil product was dissolved in 1000 ml of 1 M methanolic HCl. A six hours standing at 15° C. gives a complete cleavage of all ester groups. After evaporation the product was purified by a column chromatography on Amberlyte IR-200 (3% methanolic ammonia). Total yield of D-I: 92 percent.

Method B: Tert.-Butyl Iodoacetate and N-ethyl-N,N-diisopropylamine in DMF

D-I was prepared from C-I by same procedure and scale, as it has been described in this Example—Method A. tert.-butyl bromoacetate was placed by tert.-butyl iodoacetate. Total yield of D-I: 84 percent.

Method C: Tert.-Butyl Bromoacetate and Potassium Carbonate in DMF

D-I was prepared from C-I by same process and scale, as it has been described in this Example—Method A. N-methyl-N,N-diisopropylamine was placed by equivalent of dry and well powdered potassium carbonate. Total yield of D-I: 75 percent.

Method D: Tert.-Butyl Bromoacetate and Cesium Fluoride in DMF

D-I was prepared from C-I by same process, as it has been described in this Example—Method A. Scale was reduced to one tenth and N-methyl-N,N-diisopropylamine was placed by equivalent of dry and well powdered cesium fluoride. Total yield of D-I: 79 percent.

Method E: Benzyl Bromoacetate and N-methyl-N,N-diisopropylamine in DMF

D-I was prepared from C-I by same process and scale, as it has been described in this Example—Method A. tert.-butyl bromoacetate was placed by benzyl bromoacetate. After chromatography on Silica, benzylic ester groups were cleavage by 5 h stirring in a mixture of 1300 ml of anhydrous methanol, (41 g; 0.8 mol) of 98% hydrazine hydrate and 2 g of 10% palladium on charcoal. After evaporation the product was purified by a column chromatography on Amberlyte IR-200 (3% methanolic ammonia). Total yield of D-I: 83 percent.

Method F: Tert.-Butyl Iodoacetate and N-methyl-N,N-diisopropylamine in N-methylpyrrolidone

D-I was prepared from C-I by the same procedure and scale, as it has been described in this Example—Method A. tert.-butyl bromoacetate was placed by tert.-butyl iodoacetate and all solutions were prepared in dried N-methylpyrrolidone. Total yield of D-I: 93 percent.

Method G: Tert.-Butyl Iodoacetate and N-methyl-N,N-diisopropylamine in N,N-dimethylacetamide

D-I was prepared from C-I by the same procedure and scale, as it has been described in this Example—Method A. tert.-butyl bromoacetate was placed by tert.-butyl iodoacetate and all solutions were prepared in dried and freshly distilled N,N-dimethylacetamide. Total yield of D-I: 98 percent.

Method H: Bromoacetic Acid and N-methyl-N,N-diisopropylamine in DMF

D-I was prepared from C-I by the same procedure and scale, as it has been described in this Example—Method A. tert.-butyl bromoacetate was placed by bromoacetic acid and by equivalent of N-methyl-N,N-diisopropylamine were used. Total yield of D-I: 82 percent.

Method I: Lithium Bromoacetate in DMF

To a solution of (2.4 g; 17.24 mmol) of bromoacetic acid in 70 ml of well-dried DMF at −5° C. was added pulverized lithium hydride (143 mg; 18 mmol) free of mineral oil spots. After hydrogen evolution was completed, the solution has been added to a solution of (630 mg; 2.5 mmol) of C-I in dried DMF (20 ml) after a period of 20 minutes. During the addition, the temperature spontaneously raised to 35° C. The mixture is warmed to 50° C. After 2 hours stirring at this temperature the reaction mass is diluted by water and this mixture is twice chromatographed on column with Amberlyte IRC-50 (5% methanolic ammonia) and thereafter by column chromatography on Amberlyte IR-200 (3% methanolic ammonia). Total yield of D-I: 72 percent.

Method J: Lithium Iodoacetate in DMF

D-I was prepared from C-I by the same procedure and scale, as it has been described in this Example—Method I. Lithium bromoacetate was placed by lithium iodoacetate. Total yield of D-I: 90 percent.

Method K: Lithium Chloroacetate in DMF

D-I was prepared from C-I by the same procedure and scale, as it has been described in this Example—Method I. Lithium bromoacetate was placed by lithium chloroacetate. Total yield of D-I: 86 percent.

Method L: Lithium Iodoacetate in Water

(2.79 g; 15 mmol) of iodoacetic acid is suspended in 25 ml of water. This mixture is cooled to 0° C. At this temperature is added (1.33 g; 18 mmol) of lithium carbonate and the mixture is stirred until all lithium carbonate is dissolved. At laboratory temperature, this solution is added in one portion to solution of (534 mg; 2.14 mmol) of C-I in 5 ml of water. After two hours of stirring, this reaction mixture is processed by chromatography as it has been described in this Example—Method I. Total yield of D-I: 97 percent.

Method M: Lithium Iodoacetate in Aqueous Ethanol

D-I was prepared from C-I by the same procedure and scale, as it has been described in this Example—Method L. Aqueous milieu was placed by aqueous-ethanolic (20/80, Vol./Vol.). Total yield of D-I: 84 percent.

Method N: Calcium Iodoacetate in Water

In a mechanically stirred apparatus equipped by a thermometer and adding funnel, (1.8 g; 18 mmol) of a freshly reprecipitated calcium carbonate in 20 ml of water were suspended. To this suspension 15 mmol of iodoacetic acid were added at laboratory temperature portionwise. The mixture was vigorously stirred. To this suspension was added (534 mg; 2.14 mmol) of C-I in 5 ml of water. Temperature is rising up to 42° C. spontaneously. After one hour of stirring the temperature raised to 55° C. with continuous 4 hours stirring. Viscous suspension is diluted with 40 ml of methanol and filtered through G4, a solid washed with methanol. Aqueous phase is eluted on a column of Dowex-50W and an eluted phase is concentrated in vacuo. A trituration with ethanol-diethylether (1:1, Vol./Vol.) at 3-5° C. affords brownish impure crystalline product. Purification on Amberlyt IR-200 column affords D-I in high purity (more than 99.7%; HPLC). Total yield of D-I: 87 percent.

Method O: Magnesium Bromoacetate in Water

D-I was prepared from C-I by the same procedure and scale, as it has been described in this Example—Method N. Calcium iodoacetate was substituted by magnesium bromoacetate prepared from bromoacetic acid and an active magnesium oxide. Total yield of D-I: 72 percent.

Method P: Barium Iodoacetate in Water

D-I was prepared from C-I by the same procedure and scale, as it has been described in this Example—Method N. Calcium iodoacetate was substituted by barium iodoacetate prepared from bromoacetic acid and an active barium carbonate. After the main reaction was finished, 50 ml of methanol were added. The mixture is filtered (G4). To aqueous phase is added 40% sulphuric acid drop by drop with a potentiometric indication of sulphate anion. A slurry mixture is filtered. Filtrate is evaporated in vacuo and after dissolving in 30 ml of water, this solution is chromatographed on Dowex-50W column. Total yield of D-I: 92 percent.

Method Q: Strong Basic Annex in an Iodoacetate Cycle in Methanol

D-I was prepared from C-I by the same procedure and scale, as it has been described in this Example—Method N. Amberlite IRA-402 in iodoacetate cycle substitutes calcium iodoacetate. Aqueous milieu was replaced by a methanolic. Total yield of D-I: 85 percent.

Table 4 summarizes the results of carboxymethylation of triamine C-(I-XXI) a-d.

TABLE 4

| | Substance a | Substance b | Substance c | Substance d |
|---|---|---|---|---|
| | Method/ | Method/ | Method/ | Method/ |
| Carbon-backbone | Yield (%) | Yield (%) | Yield (%) | Yield (%) |
| D-I (R₃ = Me) | A/85 | O/87 | A/83 | N/94 |
| D-V (R₃ = Et) | G/94 | I/82 | H/80 | M/95 |
| D-VI (R₃ = Pr) | C/84 | D/86 | L/84 | I/90 |
| D-VII (R₃ = Bu) | A/86 | B/83 | C/82 | A/82 |
| D-VIII (R₃ = hexyl) | A/86 | B/83 | C/80 | A/82 |
| D-IX (R₃ = octyl) | A/85 | A/83 | D/81 | A/80 |
| D-X (R₃ = decyl) | C/86 | Q/92 | E/93 | C/85 |
| D-XI (R₃ = tetradecyl) | A/87 | A/94 | A/80 | D/89 |
| D-XII (R₃ = hexadecyl) | A/86 | B/83 | C/80 | A/82 |
| D-XIII | A/85 | K/95 | D/91 | A/80 |
| (R₃ = cyclohexylmethyl) | | | | |
| D-XIV (R₃ = p- | C/88 | I/91 | C/94 | C/82 |
| nitrobenzyl) | | | | |
| D-XV (R₃ = p- | A/90 | J/95 | A/96 | D/83 |
| methoxybenzyl) | | | | |
| D-XVI (R₃ = benzyl) | C/87 | C/92 | J/84 | L/89 |
| D-XVI (R₃ = p- | F/81 | A/93 | D/83 | M/85 |
| iodobenzyl) | | | | |
| D-XVII (R₃ = CH₂OBz) | O/93 | F/93 | C/88 | P/91 |
| D-XVIII (R₃ = CH₂SBz) | A/83 | G/90 | A/95 | Q/89 |
| D-XIX (R₃ = CH₂CH₂OH) | D/84 | C/94 | P/84 | C/88 |
| D-XX | A/91 | L/82 | C/89 | D/87 |
| (R₃ = CH₂CH₂CH₂OH) | | | | |
| D-XXI | D/85 | P/91 | A/88 | B/84 |
| (R₃ = CH₂CH₂CH₂CH₂NHBoc) | | | | |

### Example 17

### Procedure for Preparation of N-[(1S)-2-amino-1-methyl-ethyl]-N-[(1S)-2-amino-1-(4-nitrobenzyl)ethyl]amine-N,N,N′,N″,N″-pentaacetic acids (D-I-XXIb) by a Glyoxylic Acid Method

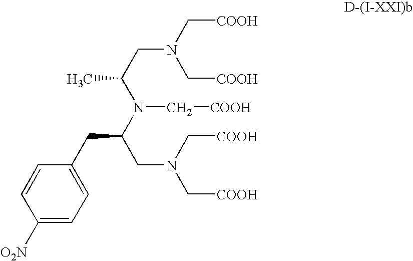

Into a 250 ml three necked reaction vessel equipped with an addition funnel (with servo and pressure correction), electronic temperature meter bonded to thermostat, nitrogen overpressure inlet adapter and strong stirring apparatus, (7.39 g; 29.3 mmol) of C-I and (15 g; 91.4 mmol) of benzyl glyoxylate in 100 ml of dried ethanol were placed. The mixture was cooled down to 8° C. (5.52 g; 88 mmol) of sodium cyanoborohydride were added portion-by-portion over period of 2 hours thereafter. After the adding was complete, temperature was raised to 20° C. Thereafter mixture was filtered and acidified with ice aqueous acetic acid to pH 6. After 24 hours standing at −5° C. it has been filtered once more. Now, the reaction mass is concentrated at vacuo (12 kPa) to 30 ml approximately. The product is precipitated by adding of 800 ml of water, filtered and dried. Benzylic ester groups were cleavage by 24 h stirring in a mixture of 330 ml of anhydrous methanol, (4.85 g; 95 mmol) of 98% hydrazine hydrate and 0.7 g of 10% palladium on charcoal. After filtration and evaporation the product was purified by a column chromatography on Amberlyte IR-200 (3% methanolic ammonia). Total yield of D-I: 87 percent.

### Example 18

### Procedure for Preparation of N-[(1R)-2-amino-1-methyl-ethyl]-N-[(1S)-2-amino-1-(4-nitrobenzyl)ethyl]amine-N,N,N′,N″,N″-pentaacetic acid (D-Id) by a Cyanhydrine Carboxymethylation

Into a 800 ml reaction bottle equipped with two adding inputs from dual peristaltic pump, temperature meter, reflux condenser with nitrogen overpressure inlet adapter and a strong mechanic stirrer apparatus were placed 175 ml of water and slurry mixture from 8 mol. % of tetrabutylammonium hydrogensulphate and (719 mg; 4.5 mmol) of sodium hydrogenphosphate hydrate and 20 ml of water. With continuous vigorous stirring (126 mg; 0.5 mmol) of C-Id, 337 mg of aqueous 40% formaldehyde solution (4.5 mmol) was added and (382 mg; 4.5 mmol) of 2-hydroxyisobutyronitrile has been added thereafter. 160 ml of 72% aqueous sulphuric acid were added within a 45 minutes. Temperature was spontaneously raised to 55° C. and the reaction mass was stirred in this temperature for a next five hours. Thereafter temperature was raised up to 85° C. and reaction mass was stirred for 6 hours. Mixture is cooled to laboratory temperature and alkalized by potassium carbonate to a strong basic reaction. After 24 hours cooling at −5° C. separated inorganic slats were filtered (G3) and washed with methanol. Liquid phase was concentrated at vacuo and acidified with 21% aqueous hydrogen chlorine. Separated crude product was filtered. Obtained solid matter was dissolved in minimum quantity of water and the product was purified by a column chromatography on Amberlyte IR-200 (3% methanolic ammonia). Total yield of D-Id: 76 percent.

### Example 19

### Procedure for Preparation of N-[(1R)-2-amino-1-methyl-ethyl]-N-[(1R)-2-amino-1-(4-nitrobenzyl)ethyl]amine-N,N,N,′N″,N″-pentaacetic acid (D-Ic) by an Enhancing Asymmetric Catalysis N-Alkylation

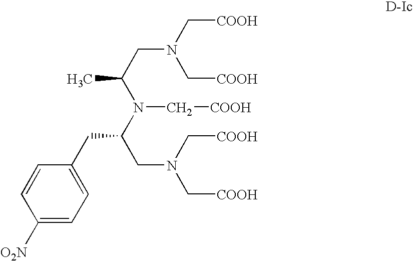

In 100 flask equipped by magnetic stirrer there were dissolved (5.04 g; 20 mmol) of a diastereomer mixture (crude from a synthesis) C-Ic/C-Id in 25 ml of dry dimethylformamide (DMF) (freshly distilled at vacuo from calcium hydride). With continuous vigorous stirring (19.3 g; 150 mmol) of N-ethyl-N,N-diisopropylamine (of a purity better than 99%) and 2 g of N-benzylcinchoninium bromide is added. (29.3 g; 150 mmol) of tert.-butyl bromoacetate in 30 ml of dried dimethylformamide were added to the solution at 25° C. over the period of 300 minutes. After addition, the temperature was slowly raised up to 65° C. and this mixture has been stirred at 65° C. under nitrogen overpressure for 48 hours. Thereafter, reaction mass was poured into 100 ml of 15° C. water with vigorous stirring. A separated oily product was extracted by 4×100 ml of dichloromethane. After evaporation is the product chromatographed on Silica (tert.-Butanol-dichloromethane, 2:3 mixture). A fraction strongly enhanced by D-Ic ester was collected. After evaporation of a separated fraction, brownish yellow oil product was dissolved in 300 ml of 1 M methanolic HCl. A ten hours standing at 25° C. gives a complete cleavage of all ester groups. After evaporation the product was purified by a column chromatography on Amberlyte IR-200 (3% methanolic ammonia). Total yield of D-Ic: 86 percent (ee 94%).

### Example 20

### Procedure for Preparation of N-[(1R)-2-amino-1-methyl-ethyl]-N-[(1R)-2-

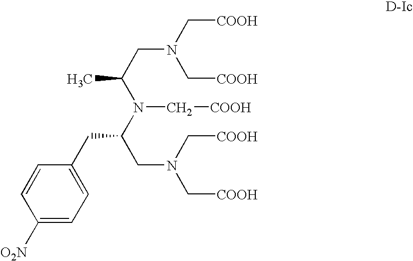

### amino-1-(4-nitrobenzyl)ethyl]amine-N,N,N′,N″,N″-pentaacetic acid (D-Ic) by a Diastereomer Separation on a Chiral Column

The reaction was carried out under the same conditions and scale as it has been described in Example 19. But no asymmetric catalysis (N-benzylcinchoninium bromide) was used. A reaction mixture obtained in those conditions wasn't separated at Silica, but it was only flash chromatographed on Silica to crude D-Ic/D-Id mixture separation. The mixture was separated on an Aza-MDS chiral column (mobile phase: ethyl acetate-dichloromethane, 1:1, Vol./Vol.). Total yield of D-Ic: 79 percent (ee 94%).

### Example 21

### Procedure for Preparation of N-[(1R)-2-amino-1-methyl-ethyl]-N-[(1R)-2-

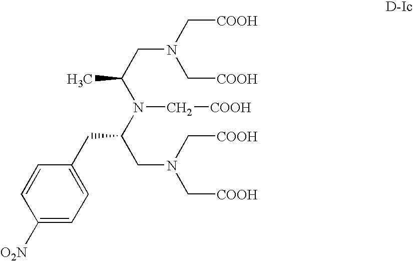

### amino-1-(4-nitrobenzyl)ethyl]amine-N,N,N′,N″,N″-pentaacetic acid (D-Ic) by a Diastereomer Separation

D-Ic was prepared from C-Ic/C-Id diastereomer mixture by the same procedure and scale, as it has been described in this Example 20. The mixture of diastereomers was separated by recrystallization with (+)-dehydroabietylamine (purity of min. 98%) in anhydrous methanol. Total yield of D-Ic: 91 percent.

### Example 22

### Procedures for Preparation of (D-XXIIb)

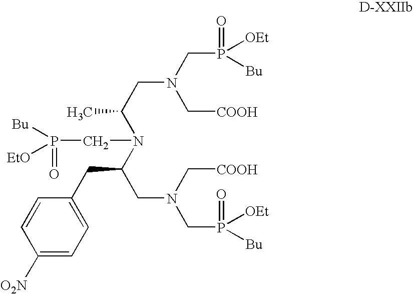

3.78 g of C-Ib (15 mmol) was dissolved in the mixture prepared from a 60 ml dimethylformamide (DMF), mol) 4.84 g of potassium carbonate (35 mmol) and 2 g of tetrabutylammonium hydrogen sulphate. Within 10 minutes 5.48 g of bromomalonic acid (30 mmol) was added. Reaction mixture is heated up to 50° C. and in this temperature is viscous mass stirred for 6 hours. After cooling, insoluble salts are well filtered. After evaporation of DMF below 50° C. at vacuo, crude product is dissolved in 60 ml of 30% sulphuric acid at hot. This mixture is warmed to 90° C. for 60 minutes. After cooling to 55° C. excess of an active barium carbonate is added. Slurry matter is diluted with 300 ml of warm water and filtered (G4). Obtained filtrate is evaporated at vacuo. Brown mass is dried over phosphorus pentoxide. Dry intermediate is dissolved in dry acetonitrile (230 ml). To this mixture is added solution 15 g of (butyl-ethoxy-phosphinoylmethyl)-trimethyl-ammonium bromide 50 mmol) in dry acetonitrile (120 ml) at room temperature. Mixture was refluxed under nitrogen for 6 hours. The solvent was removed in vacuo, and the residue was partitioned between dichloromethane (50 ml) and 10% aqueous NH₄Cl (15 ml). The organic phase was extracted with water (10 ml), dried over fresh mol. sieve and the solvent removed in vacuo. Total yield: 82 percent.

### Example 23

### General Procedures of Protection Terminal Amino Groups of C-(I-XXI)a-d

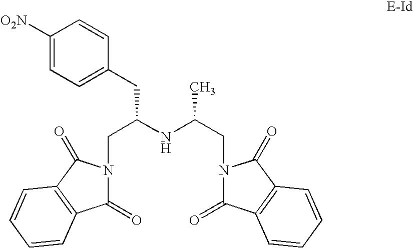

A mixture 252 mg of triamine C-Id (1 mmol) and 296 mg of phthalic anhydride (2 mmol) in 15 ml of glacial acetic acid was refluxed for 1.5 h. Solvent was removed on a rotary evaporator and was replaced with 20 ml of hot 2-propanol with stirring until a solid appeared. The product was collected and washed with cold 2-propanol. Total yield of E-Id: 82 percent.

### Example 24

### General Procedures of N-Alkylation of Secondary Nitrogen of Diprotected Nitroaminobenzyldiamine E-Id

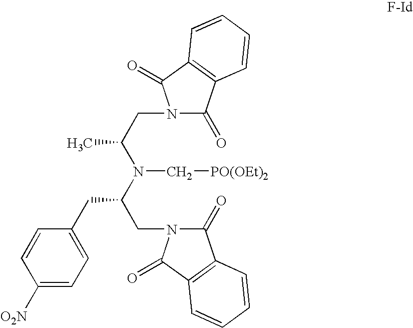

Method A

E-Id (768 mg; 1.5 mmol) and triethyl phosphite (310 mg; 1.87 mmol) was introduced into a flask and the flask was immersed in an ice bath. Paraformaldehyde (66 mg; 2.2 mmol) was added in small portions over a period of 30 min. The mixture was then allowed to warm up to room temperature and stirring was continued for 4 days at room temperature and 1 day at 50° C. The clear mixture was kept under high vacuum at 40-50° C. for several hours to remove volatile impurities. Total yield of F-Id: 72 percent.

### Example 25

### General Procedures of N-Alkylation of Secondary Nitrogen of Diprotected Nitroaminobenzyldiamine E-I(a-d)

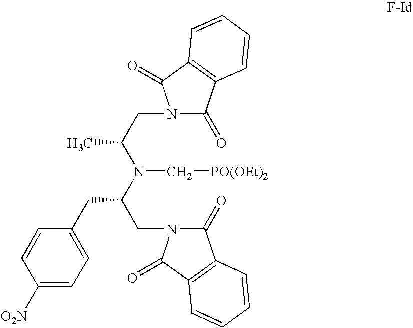

Method B

E-Id (768 mg; 1.5 mmol) and diethylphosphate (549 mg; 4.5 mmol) was dissolved in the flask in the solution of toluene and ethanol (3:1). A suspension of toluene and a dry paraformaldehyde (180 mg; 6 mmol) in small portions during 1 h was added to this solution, while water was removed by azeotropic distillation with Dean-Stark trap. Destillation has continued for 3 h and then the solution was evaporated under high vacuum at 60° C. to a brown oil. This oil was redissolved in anhydrous ethanol, filtered and evaporated under vacuum. This procedure is repeated three times. Product is obtained as viscous brown oil. This can be further purified by silicagel chromatography. Total yield of F-Id: 76 percent.

### Example 26

### General Procedures of N-Alkylation of Secondary Nitrogen of Diprotected Nitroaminobenzyldiamine E-I(a-d)

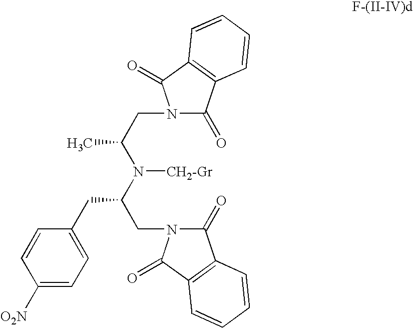

Compound F-(II-IV)d was prepared from E-Id by the same method as it has been described in Example 25 with esters alkylphosphinate on place of diethylphosphate. See Table 5.

TABLE 5

| compounds | ester alkylphosphinate | Produkt Gr | Yield [%] |
|---|---|---|---|
| F-IId | HPO(OEt)Me | —PO(OEt)Me | 72 |
| F-IIId | HPO(OEt)CH₂CH₂CH₂NHBoc | —PO(OEt)CH₂CH₂CH₂NHBoc | 65 |
| F-IVd | HPO(OEt)Ph | —PO(OEt)Ph | 87 |

### Example 27

### General Procedures of Deprotection Terminal Amino Groups of F-(I-Iv)a-d

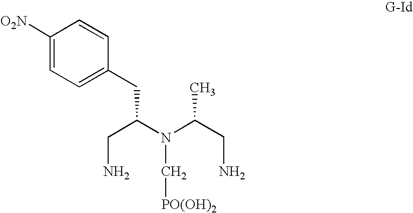

A suspension of F-Id (331 mg; 0.5 mmol) in hydrochloric acid (6 M, 30 ml) was refluxed for 24 h. After cooling, filtering, and washing with hydrochloric acid (6 M, 4×5 ml), the combined filtrates were evaporated leaving an amorphous product, which was further dried over P₂O₅ in vacuo. Total yield of G-Id: 85 percent.

### Example 28

### General Procedures of Deprotection Terminal Amino Groups of F-(I-Iv)a-d

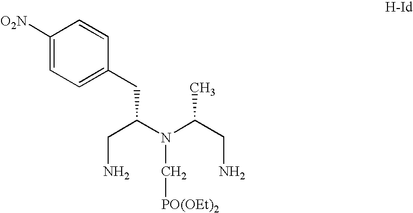

To a solution of F-Id (331 mg; 0.5 mmol) in 95% acetonitrile/water (3 ml) and hydrazine hydrate (0.25 ml) was added and the reaction mixture stirred at room temperature until HPLC analysis showed no starting material to be present (40 h). The resulting white precipitate was filtered, washed with acetonitrile, and the combined filtrates were evaporated using a rotary evaporator at room temperature under high vacuum to give pure products, which was further dried over P₂O₅ in vacuo. Total yield of H-Id: 88 percent.

### Example 29

### General Procedures of Carboxymethylation of H-I(a-d)

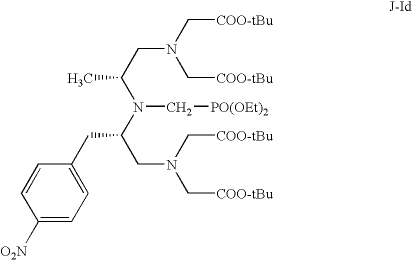

To a solution of H-Id (201 mg; 0.5 mmol) in acetonitrile (4 ml) was added 7 mol equivalents of tert.-butylbromoacetate (683 mg; 3.5 mol) and DIPEA (452 mg; 3.5 mol). The mixture was stirred at room temperature overnight, then refluxed for 3.5 h. The solvent was then evaporated to dryness and the residual oily product dissolved in dichloromethane (3 ml), which was then washed with 10% citric acid, sodium hydrogen carbonate (1 M) and demineralized water. After drying the organic layer over Na₂SO₄, filtration and evaporation gave an oily product which was chromatographed on a silicagel column. Product was obtained as yellow oil. Total yield of J-Id: 58 percent.

### Example 30

### General Procedures of Hydrolysis of J-I(a-d)

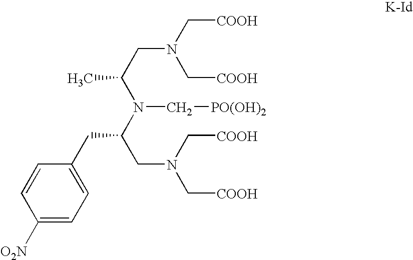

Tetra-tert.-Butyl ester (200 mg; 0.23 mmol) was refluxed and stirred in 6 ml 8 M HCl during 24 h. Evaporation of the solvent, followed to a solid and loaded onto an ion-exchange column of AG 50W-X8, 200-400 mesh, H⁺ form, and washed with H₂O to remove the hydrolysis products. The crude product was eluted with 1.8 N aqueous NH₃. Eluents containing product were combined and evaporated at vacuo. After chromatography purification on Amberlite CG-50 (H⁺-form) column there have been obtained product as free acid. Total yield of K-Id: 80 percent.

### Example 31

### General Procedures of Reductions Nitrobenzyl-Ligands D-(I-XXII)a-d to Aminobenzyl-Ligands N-(I-XXII)a-d

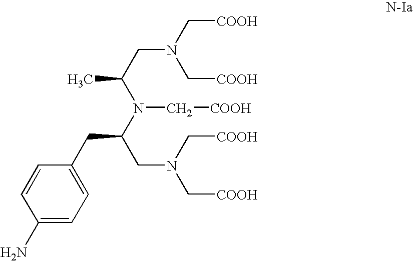

A 5 g of nitrobenzyl-ligand D-Ia (5 g; 9.2 mmol) was dissolved in 100 ml demineralized H₂O and 500 mg of 10% Pd/C. Suspension was then stirred at the room temperature and the flow of gaseous hydrogen was introduced under the surface of the solution. Reaction was monitored by TLC analysis until the starting nitroligand in the reaction mixtures cannot be detected (1-7 days). The contents of flask was filtered through a fine frit coated with Celite. The filtrate was concentrated under vacuum to dryness. Thus, aminobenzyl-ligand N-Ia in almost quantitative yield was obtained as a yellowish glassy product. Total yield of N-Ia: 98 percent.

### Example 32

### General Procedures of Hydrolysis of J-(II-IV)d

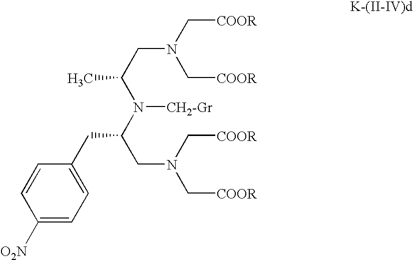

Compounds K-(II-IV)d were prepared from reactant by same method as it has been described in Example 31 with 8 M HCl. See below.

| | | | Yield |
|---|---|---|---|
| compounds | reactant Gr/R | product Gr/R | [%] |
| K-IId | —PO(OEt)Me/t-Bu | —PO(OH)Me/H | 95 |
| K-IIId | —PO(OEt)CH₂CH₂CH₂NH₂/Et | —PO(OH)CH₂CH₂CH₂NH₂/H | 82 |
| K-IVd | —PO(OEt)Ph/t-Bu | —PO(OH)Ph/H | 96 |

### Example 33

### General Procedures of Reduction K-(II-IV)d

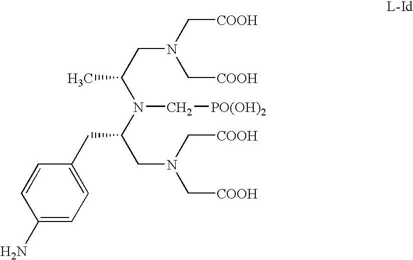

A 5 g of nitrobenzyl-ligand K-Id (5.26 g; 9.1 mmol) was dissolved in 100 ml demineralized H₂O and 520 mg of 10% Pd/C. Suspension was then stirred at room temperature and the flow of gaseous hydrogen was introduced under the surface of the solution. Reaction was monitored by TLC analysis until the starting nitroligand in the reaction mixtures cannot be detected (1-7 days). The contents of the flask were filtered through a fine frit coated with Celite. The filtrate was concentrated under vacuum to dryness. Thus, aminobenzyl-ligand L-Id in almost quantitative yield was obtained as a yellowish glassy product. Total yield of L-Id: 95 percent.

### Example 34

### General Procedures of Preparations ITC-Derivates of Aminobenzyl-Ligands M-(I-XXII)a-d

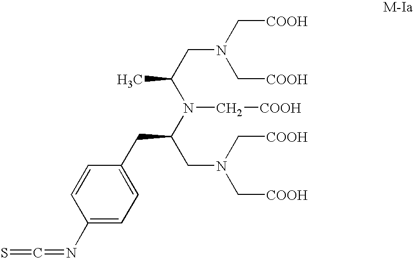

The aminobenzyl-ligand N-Ia (169 mg; 0.33 mmol) was taken up in 10 ml demineralized H₂O and stirred rapidly in flask fitted with an addition funnel. The pH was adjusted to 8.5 with solid NaHCO₃, and thiophosgene (43 mg, 0.37 mmol) in 10 ml chloroform was added dropwise. Stirring was continued until the solution tested negative for amine by the fluorescamine. The aqueous layer was washed with chloroform (4×5 ml) and then. Purification was done by column chromatography on Florisil column eluted with acetonitrile-H₂O. The fraction with product was lyophilized and stored in a desiccator in a freezer.

### Example 35

### General Procedures of Preparations α-Bromoacetamido Derivates of Aminobenzyl-Ligands

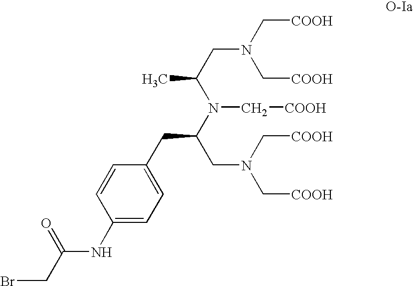

Aminobenzyl-ligand N-Ia (256 mg; 0.5 mmol) was dissolved in 5 ml of water. The pH was adjusted to 7-8 using diisopropylethylamine. This solution was added dropwise to a stirring solution of bromoacetyl bromide (0.5 g; 2.5 mmol) in 5 ml of chloroform. The pH of the resulting solution was adjusted to 7.0 with diisopropylethylamine and stirred vigorously for 5 min. HPLC analysis of a small analytical sample revealed that the reaction had gone to completion by the disappearance of the starting material peak and the appearance of a new peak. The layers were separated, and the aqueous phase was extracted with chloroform. The pH of the aqueous phase was adjusted to 7-8 with diisopropylethylamine and extracted with chloroform. This was repeated four more times. The pH of the aqueous phase was adjusted to 1.5-1.8 with 3 M HCl and extracted twice with equal volumes ethyl ether. The pH was readjusted with 3 M HCl and the aqueous phase extracted twice with ethyl ether. This was continued until the pH remained constant. Residual ether was removed from the aqueous solution under reduced pressure. The pH of the solution was adjusted to 4.5 with 3 M NaOH, and the solution was divided into aliquots, frozen in liquid nitrogen, and stored at −70° C.

### Example 36

### General Procedures of Preparations α-Bromoacetamido Derivates of Aminobenzyl-Ligands

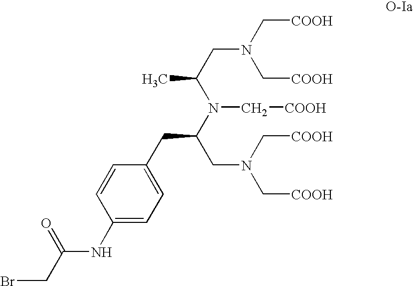

Aminobenzyl-ligand penta-tert.-butyl ester of N-Ia-5tBu (79 mg; 1 mmol) was dissolved in 10 ml dichloromethane in a three-necked flask equipped with a magnetic stirring apparatus under argon. Two addition funnels, each containing 7 ml of dichloromethane were attached to the flask. Anhydrous DIEA (258 mg; 2 mmol) was added to one funnel and bromoacetyl bromide (303 mg; 1.5 mmol) was added to the other. The DIEA and bromoacetyl bromide were added to the flask simultaneously with stirring over 10 min. The mixture was allowed to stir at room temperature for 5 min under argon. The mixture was directly loaded on gel column and purified. Product-containing fractions were evaporated. Thus, bromoacetamidobenzyl-ligand penta-tert.-butyl ester in almost quantitative yield was obtained as a yellowish glassy product. This substance was deprotected by overnight mixing in anhydrous trifluoroacetic acid under inert atmosphere. Evaporated solvent under reduced pressure yielded an amorphous solid of bromoacetamidobenzyl-ligand pentaacetic acid.

### Example 37

### General Procedures of Preparations α-Iodoacetamido Derivates of Aminobenzyl-Ligands

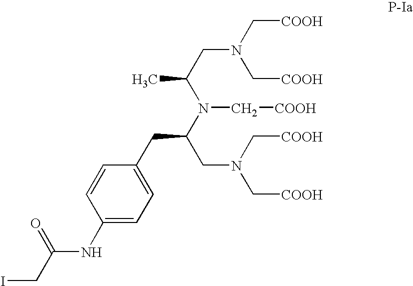

Compound P-Ia was prepared from aminobenzyl-ligand penta-tert.-butyl ester N-Ia-5tBu by same method as it has been described in Example 35 with iodoacetyl chloride on place of bromoacetyl bromide.

### Example 38

### General Procedures of Maleimidoalkylcarboxamidation of Aminobenzyl-Ligands

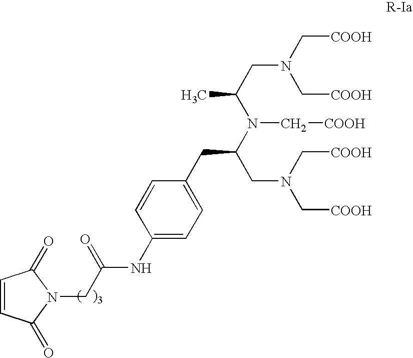

Aminobenzyl-ligand N-Ia (44.6 mg; 0.087 mmol) was dissolved in 0.5 ml of dimethylformamide (DMF) to give a yellow solution. Triethylamine (96 mg; 0.95 mmol) was added to this, which changed the reaction mixture (pH 8) from pink to off-white. γ-Maleimidobutanoic acid N-hydroxysuccinimide ester (67 mg; 0.24 mmol) was dissolved in 0.5 ml of DMF and added to the reaction mixture. A yellow solution was obtained, and a white precipitate settled to the bottom. The mixture was allowed to stand for 3 h at room temperature with occasional stirring. The precipitate formed was filtered and the filtrate was evaporated to dryness in vacuum. The impurities were removed by washing with chloroform and methanol. The residue was purified by Sephadex LH-20 column.

### Example 39

### General Procedures of Maleimidoalkylcarboxamidation of Aminobenzyl-Ligands

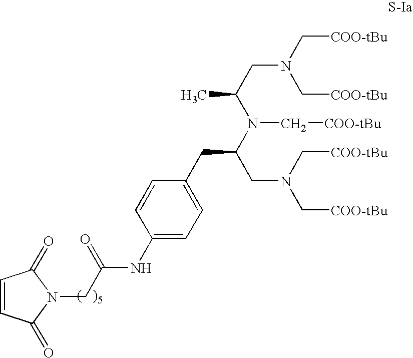

Compounds S-Ia was prepared from aminobenzyl-ligand penta-tert.-butyl ester N-Ia-5tBu by same method as it has been described in Example 38 with ε-maleimidocaproic acid N-hydroxysuccinimide ester place of γ-Maleimidobutanoic acid N-hydroxysuccinimide ester.

### Example 40

### General Procedures of Vinylcarboxamidation of Aminobenzyl-Ligands

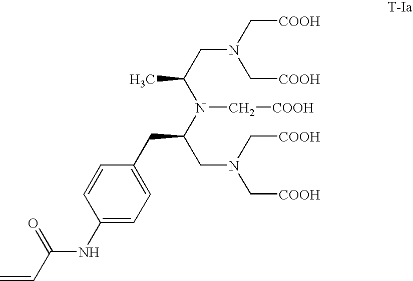

Aminobenzyl-ligand penta-tert.-butyl ester N-Ia-5tBu (1.26 g; 1.6 mmol) was dissolved in 10 ml dichloromethane in a three-necked flask equipped with a magnetic stirring apparatus under argon. Two addition funnels, each containing 5 ml of dichloromethane were attached to the flask. Anhydrous DIEA (416 mg; 3.22 mmol) was added to one funnel and acryloyl chloride (217 mg; 2.4 mmol) was added to the other. The DIEA and acryloyl chloride were added to the flask simultaneously with stirring over 10 min. The mixture was allowed to stir at room temperature for 5 min under argon. The mixture was directly loaded on gel column and purified. Product-containing fractions were evaporated. Thus, acrylamidobenzyl-ligand penta-tert.-butyl ester in almost quantitative yield was obtained as a yellowish glassy product. This substance was deprotected by overnight mixing in anhydrous trifluoroacetic acid under inert atmosphere. Evaporated solvent under reduced pressure yielded an amorphous solid of acrylamidobenzyl-ligand pentaacetic acid T-Ia.

### Example 41

### General Procedures of Vinylsulfonylamidation of Aminobenzyl-Ligands

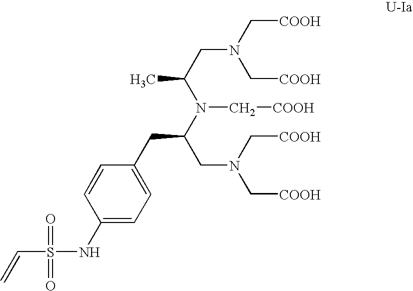

To a solution of 2-chloroethanesulfonyl chloride (538 mg; 3.3 mmol) in 31 ml of DMF that was cooled in an ice-water bath, was added aminobenzyl-ligand N-Ia (1.69 g; 3.3 mmol) and triethylamine (364 mg; 6.3 mmol), respectively. The resulting mixture was stirred at 0° C. for 1 h and a second batch of triethylamine (3.6 mmol) was added. Reaction mixtures were evaporated in high vacuum on RVO. The residue was then poured onto a mixture of 10% NaHSO₄ and ice, followed by addition of more methylene chloride. The organic phase was separated and the aqueous phase extracted with methylene chloride. The aqueous phase was separated and evaporated in high vacuum. Reaction mixture was purified by RP-HPLC. Chromatography provided a pure product U-Ia.

### Example 42

### General Procedures Preparations of 2-Oxoethylaminobenzyl-Ligands

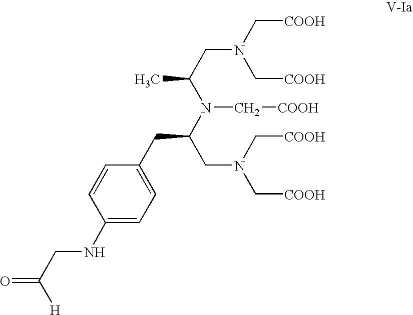

The Aminobenzyl-ligand N-Ia (128 mg; 0.25 mmol) in 1 ml of phosphate buffer (pH 7.5) was incubated at 30° C. for 2 h with glycolaldehyde (45 mg; 0.75 mmol). The solution was extracted with dichloromethane and the product purified by RP-HPLC. Fractions containing product (V-Ia) were joined and lyophilized.

### Example 43

### General Procedures of N-Vinylsulfonylethylenation of Aminobenzyl-Ligands

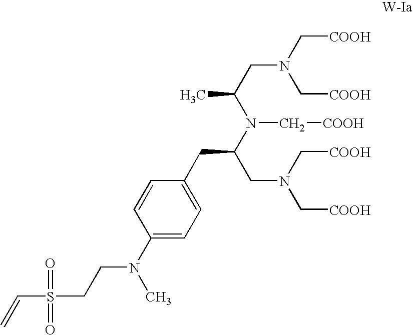

Divinyl sulfone (590 mg; 5 mmol) was dissolved in 1 ml of H₂O and 1 ml DMF, the pH was adjusted to 10 with 1 M NaOH, and N-methyl derivate of N-Ia (263 mg; 0.5 mmol) was added to 2 ml of water and reacted for 1.5 h at room temperature. The reaction mixture was loaded onto a Dowex 1-X8 (acetate) column (50 ml), washed with 50 ml of water, and eluted stepwise with 80 ml each of 0.08; 0.15 and 0.25 M acetic acid (8-10 ml fractions). Fractions containing product were joined and lyophilized.

### Example 44

### General Procedures of 6-(Vinylsulfonyl)Hexylsulfonyl Ethylation of Aminobenzyl-Ligands

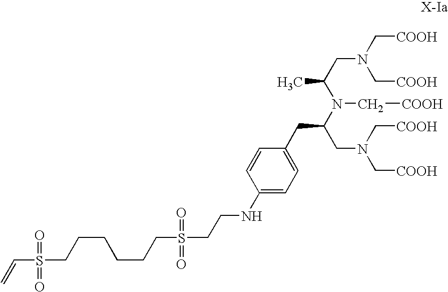

Aminobenzyl-ligand N-Ia (1.33 g; 2.6 mmol) was mixed with 1,6-hexane-bis-vinyl sulfone (6.9 g; 26 mmol) in 3 ml H₂O and 3 ml DMF, the pH was adjusted to 8.3 with 0.5 M KOH, and the reaction was run for 24 h at RT. The solution was extracted with dichloromethane and the aqueous phase was separated and evaporated in high vacuum. The raw product was purified by RP-HPLC or was loaded onto a Dowex 1-X8 (formate) column, washed with water, and eluted with gradient of water—(0.01-0.25 M formic acid). Fractions containing product were joined and lyophilized.

### Example 45

### General Procedures of 4-(2,5-dioxo-2,5-dihydro-1H-pyrrol-1-yl)benzenesulfonylation of Aminobenzyl-Ligands

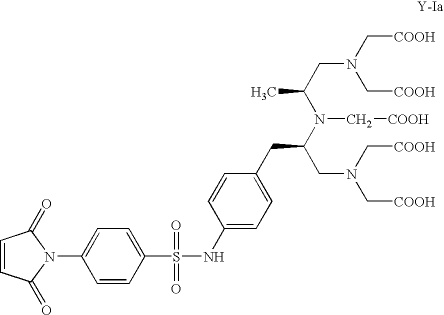

In 1 ml demineralized water was dissolved aminobenzyl-ligand N-Ia (256 mg; 0.5 mmol), the pH was adjusted to 8 with saturated Na₂CO₃. Than the solution of 4-(2,5-dioxo-2,5-dihydro-1H-pyrrol-1-yl)benzenesulfonyl chloride (103 mg; 0.6 mmol) in 1 ml of dichloromethane was added dropwise and stirred vigorously. The reaction was run for 2.5 h at RT. The organic phase was separated and the aqueous phase was extracted again with dichloromethane. The aqueous phase was liofilizated and the product was purified by RP-HPLC. Fractions containing product were joined and lyophilized, and stored at −70° C.

### Example 46

### General Procedures of 4-(N-maleimidomethyl)cyclohexane-1-carboxamidation of Aminobenzyl-Ligands

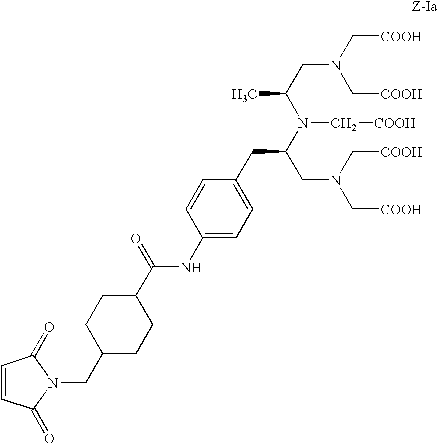

Compounds Z-Ia were prepared from aminobenzyl-ligand N-Ia by the same method as it has been described in Example 38 with 4-[(2,5-dioxo-2,5-dihydro-1H-pyrrol-1-yl)methyl]cyclohexanecarboxylic acid N-hydroxysuccinimide ester place of γ-Maleimidobutanoic acid N-hydroxysuccinimide ester.

### Example 47

### General Procedures of m-maleimidobenzoylation of Aminobenzyl-Ligands

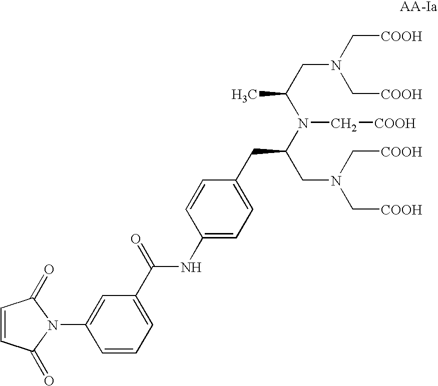

Compounds AA-Ia were prepared from aminobenzyl-ligand N-Ia by the same method as it has been described in Example 38 with 3-(2,5-dioxo-2,5-dihydro-1H-pyrrol-1-yl)benzoyl chloride place of γ-Maleimidobutanoic acid N-hydroxysuccinimide ester.

### Example 48

### General Procedures for Conjugations of Peptides with NH₂ Group

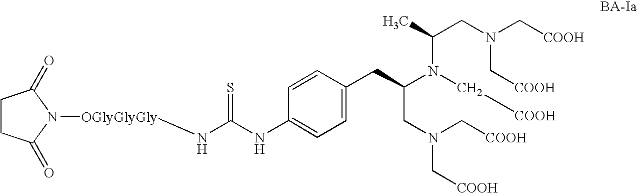

Conjugate BA-Ia was prepared by adding 3 molar excess of M-Ia in dimethylformamide (7 mg/ml) to triglycine-OSu (10 mg/ml) in borate-buffered saline (0.05 M, pH 8.5), prior to incubation at 37° C. for 20 hr. The conjugate was then purified by Sephadex G-50 column chromatography (1.8×40 cm) equilibrated and eluted with 0.1 M acetate buffer (pH 3.0). The respective conjugate fractions collected were subsequently concentrated to 5 mg/ml by ultrafiltration.

### Example 49

### General Procedures for Conjugations of Peptides with SH Group

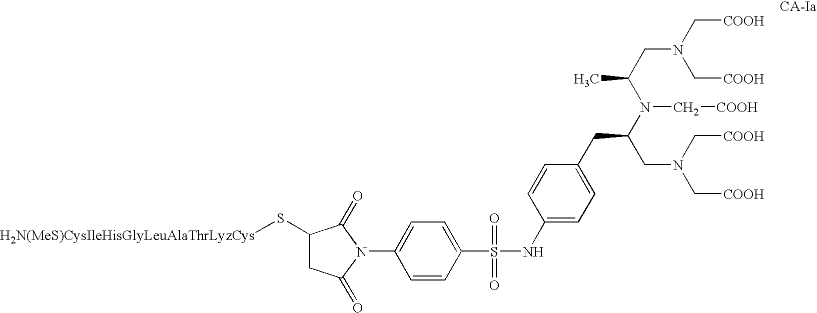

Conjugate CA-Ia was prepared by adding 3 molar excess of Y-Ia in DMSO (7 mg/ml) to SH-CysLyzThrAlaLeuGlyHisIleCys(SMe)NH₂ (10 mg/ml) in borate-buffered saline (0.05 M, pH 8.5) prior to incubation at 37° C. for 20 hr. The conjugate was then purified by Sephadex G-50 column chromatography (1.8×40 cm), equilibrated and eluted with 0.1 M acetate buffer (pH 3.0). The respective conjugate fractions collected were subsequently concentrated to 5 mg/ml by ultrafiltration.

### Example 50

### Preparation of (R)-methyl 2-((S)-1-ethoxy-1-oxopropan-2-ylamino)-3-(4-ethoxyphenyl)propanoate (Xa) and (R)-methyl 2-((R)-1-ethoxy-1-oxopropan-2-ylamino)-3(4-ethoxyphenyl)propanoate (Xb)

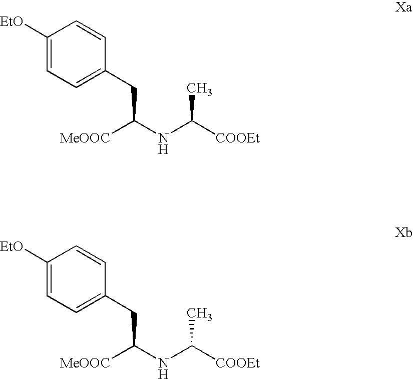

A dry K₂CO₃ (2.07 g; 15 mmol) was added to an acetonitrile solution of N-nosyl-p-ethoxy-D-phenylalanine methyl ester (3.23 g; 10 mmol and TEBA (228 mg; 1 mmol in argon inert atmosphere. The heterogeneous mixture was stirred at 55° C. and then ethyl 2-bromopropionate was added dropwise. The reaction mixture was warmed and stirred until no starting material was detectable with TLC analysis. Cooled to room temperature, diluted with water (50 ml) and extracted with dichloromethane. The organic layer was washed with water and brine, dried over Na₂SO₄ and evaporated under vacuum to give pure N-alkyl sulfonamide. Then it was added anhydrous potassium carbonate (45 mmol) to a solution of the N-alkyl sulfonamide and thiophenol (3.85 g; 35 mmol) in acetonitrile. The reaction mixture was stirred at room temperature overnight or stirred at 50° C. for 1 h. The resulting solution was reduced under vacuum and the residue taken up in diethyl ether. After usual workup the crude following esters were isolated. Esters were separated by silicagel column chromatography.

### Example 51

### Preparation of (S)-methyl 2-((S)-1-ethoxy-1-oxopropan-2-ylamino)-3-(4-ethoxyphenyl)propanoate (Xc) and (S)-methyl 2-((R)-1-ethoxy-1-oxopropan-2-ylamino)-3-(4-ethoxyphenyl)propanoate (Xd)

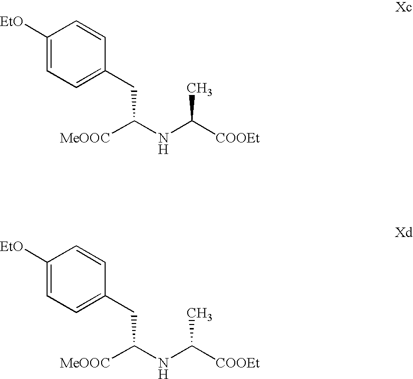

(S)-methyl 2-amino-3-(4-ethoxyphenyl)propanoate hydrochloride (1.11 g; 5 mmol) and ethyl 2-bromopropionate (7.2 g; 40 mmol) and sodium iodide (6.74 g; 45 mmol) were dissolved in 10 ml of dry DMF. Pyridine (3.16 g; 40 mmol) and silver oxide (4.63 g; 20 mmol) were added to the solution, and the mixture was stirred at room temperature for 5 h. The precipitate was filtered. H₂O (10 ml) was added to quench the reaction, and the pH was adjusted to 8-9. After concentrating the DMF-water solution by vacuum destination, the residual oil was extracted with CHCl₃. The CHCl₃ layer was dried (Na₂SO₄) and concentrated, and the residue was purified by column chromatography on silica gel.

### Example 52

### General Procedure for Preparation of (2R/S)-2-{[(1RS)-2-amino-1-methyl-2-oxoethyl]amino}-3-(4-ethoxyphenyl)propanamide (Xaa-Xdd)

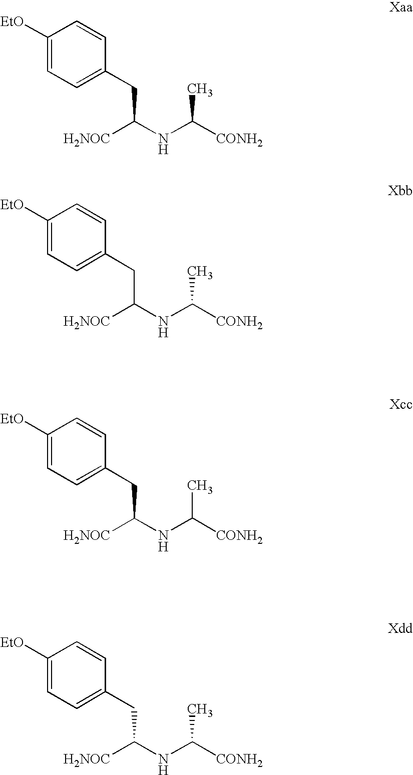

An oil of diester Xa (16.1 g; 50 mmol) was added to dry 250 ml methanol previously saturated with ammonia gas at −16° C. tightly stoppered and left at −16° C. for 14 days. After this time is the transformation quantitative (TLC analysis). The solution is carefully warmed up to laboratory temperature. A most of sorbed ammonia is forced out by the stream of nitrogen. Then the solution is evaporated at RWO under reduced pressure. It is obtained clear diamid Xaa.

### Example 53

### General Procedure for Preparation of (2R/S)-2-{[(R/S)-2-amino-1-methyl-2-oxoethyl]amino}-3-(4-ethoxyphenyl)propanamide (Xaaa-Xddd)

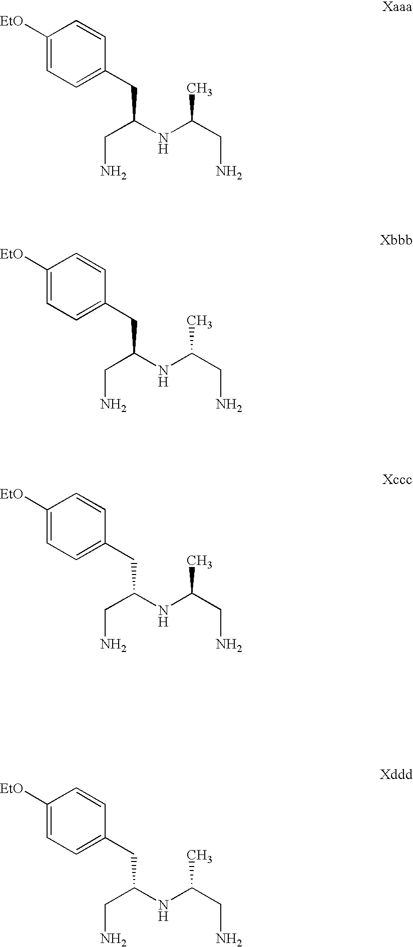

A solution of boron trifluoride etherate (852 mg; 6 mmol) in tetrahydrofurane (10 ml) was added slowly to a room temperature solution of lithium borohydride (132 mg; 6 mmol) and amide Xaa or Xbb or Xcc or Xdd (279 mg; 1 mmol) in tetrahydrofurane (25 ml) under an inert atmosphere. The mixture was heated to reflux until TLC monitoring showed complete consumption of the substrate. The reaction mixture was cooled to 0° C., quenched with water (caution: vigorous gas evolution) keeping the temperature ≦15° C. After 30 min, the THF was removed under reduced pressure. The residue dissolved in EtOH (10 ml) and 6M HCl (10 ml), and the resulting solution refluxed for 18 h. The solution was evaporated, the residue dissolved in dematerialized H₂O, and the pH of mixture adjusted with concentrated NH₄OH and 5 M KOH to 12. The aqueous solution was extracted with ten 10 ml portions of CHCl₃. The organic layer was separated, washed with brine, dried over anhydrous Na₂SO₄, and the solvent was removed under reduced pressure. Purification of the residue by silica gel chromatography gave pure amine in the indicated yields: Xaaa (89%); Xbbb (94%); Xccc (85%); Xddd (87%).

### Example 54

### Procedures for Preparation 2,2′-((R/S)-2-(((R/S)-1-(bis(carboxymethyl)amino)-3-(4-ethoxyphenyl)propan-2-yl)(carboxymethyl)amino)propylazanediyl)diacetic acid (X4a-X4d)

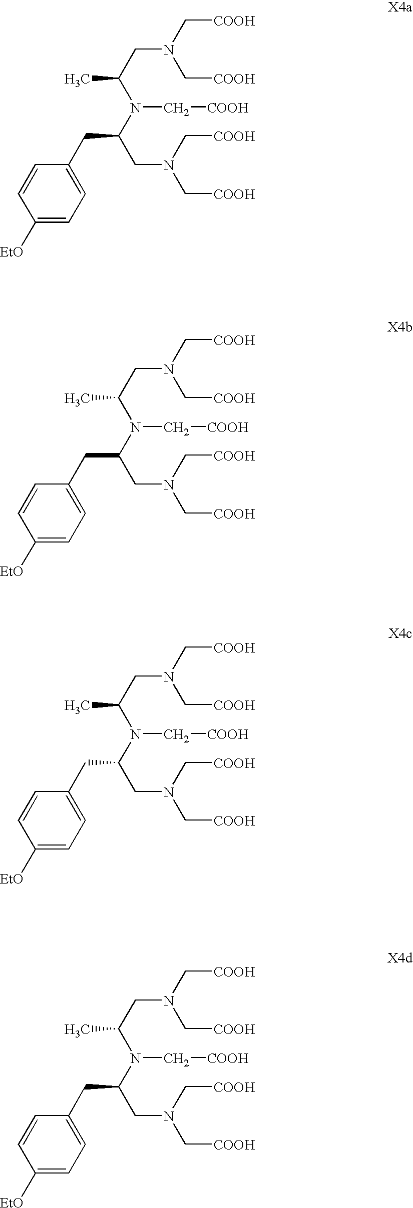

28.8 g (115 mmol) of Xaaa or Xbbb or Xccc or Xddd in 1600 ml of dried dimethylformamide (DMF) were placed into a 5 l three necked reaction vessel equipped with an addition funnel (with servo and pressure correction), electronic temperature meter bonded to thermostat, nitrogen overpressure inlet adapter and stirring apparatus. (97.9 g; 0.85 mol) of N-methyl-N,N-diisopropylamine (of a purity better than 98%) in 300 ml of dried DMF were added thereafter. (156 g; 0.8 mol) of tert.-butyl bromoacetate in 1000 ml of dried dimethylformamide were added to the solution at 25° C. over the period of 60 minutes. After addition, the temperature was slowly raised up to 65° C. and this mixture has been stirred at 65° C. under nitrogen overpressure for 24 hours. Thereafter, reaction mass was poured into 6 liters of 15° C. water with vigorous stirring. A separated oily product was extracted by 6×300 ml of dichloromethane. After evaporation is the product chromatographed on Silica (tert.-Butanol-dichloromethane, 2:3 mixture). After evaporation of appropriate fractions, brownish yellow oil product was dissolved in 1000 ml of 1 M methanolic HCl. A six hours standing at 15° C. gives a complete cleavage of all ester groups. After evaporation the product was purified by a column chromatography on Amberlyte IR-200 (5% methanolic ammonia). Total yield: X4a 89%; X4a 85%; X4b 88%; X4c 85%.

### Example 55

### Procedures for Preparation 2,2′-((R/S)-2-(((R/S)-1-(bis(carboxymethyl)amino)-3-(4-hydroxyphenyl)propan-2-yl)(carboxymethyl)amino)propylazanediyl)diacetic acid (X5a-X5d)

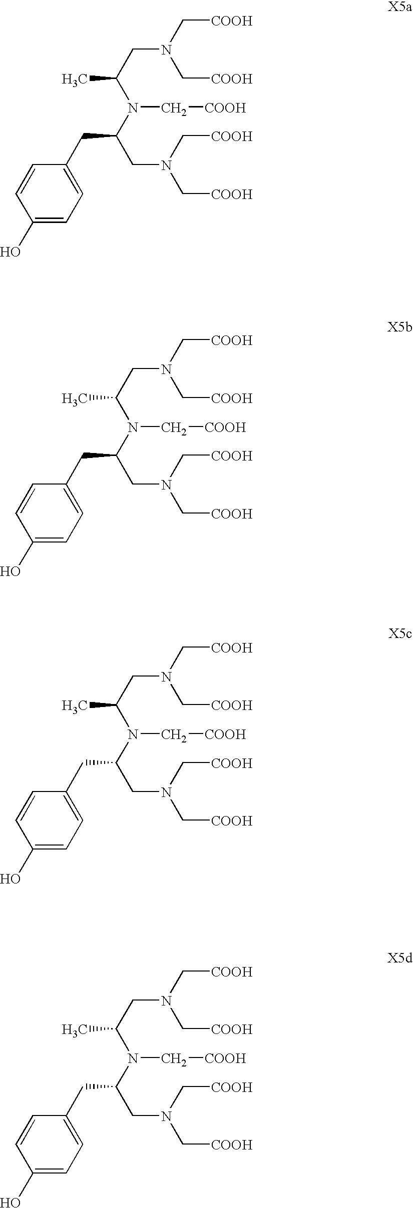

5.4 g (100 mmol; 1 eq.) of X4a or X4b or X4c or X4d was added to a solution of 5 eq Lil in 35 ml collidine and the mixture refluxed for 20 h under argon atmosphere. After cooling, 10 ml MeOH were cautiously added and the volatiles removed in high vacuo. After evaporation the product was purified by a column chromatography on Amberlyte IR-200 (3% methanolic ammonia). It is obtained an enantiomer pure X5a 55%; X5a 58%; X5b 60%; X5c 52%.

### Example 56

### General Procedures Preparations of N-2-(tert-butoxycarbonylaminooxy)acetate-ligands

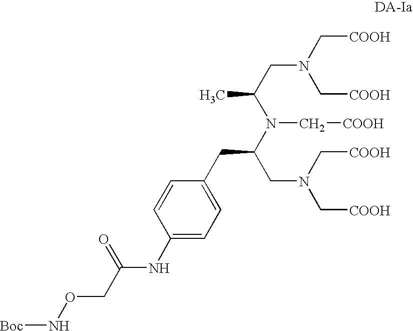

A 50-mL flask was charged with Aminobenzyl-ligand N-Ia (95.7 mg, 0.187 mmol) and dimethyl sulfoxide (DMSO) (12 mL). To this, the above 2,5-dioxopyrrolidin-1-yl 2-(tert-butoxycarbonylaminooxy)acetate (81.9 mg, 0.284 mmol) was added, and the mixture was stirred for 72 h. The DMSO was removed by high vacuum rotary evaporation to yield a clear, colorless oil. Acetonitrile (25 mL) was added and the mixture was placed at −20° C. for 24 h. The acetonitrile was decanted and the product vacuum dried to yield a powder DA-Ia (57.5 mg, 55%).

### Example 57

### General Procedures Preparations of N-2-(aminooxy)acetate Ligands EA-Ia-EA-Id

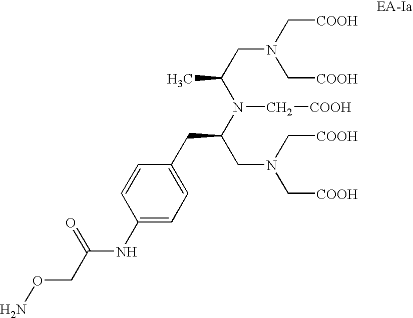

DA-1a (110 mg, 1.6 mmol) was stirred in trifluoroacetic acid (8 mL) for 24 h. The solution was then rotary-evaporated to dryness and the residue vacuum dried. After evaporation the product was purified by a column chromatography on Amberlyte IR-200 (3% methanolic ammonia). It is obtained an enantiomer pure EA-Ia as a light yellow powder (78%).

### Example 58

### General Procedures Preparations of N-carboxymethyl-ligands FA-Ia-FA-Id

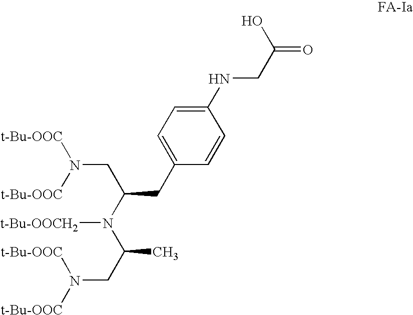

To a suspension of penta tert-butyl ester of D-Ia (7.9 g; 10 mmol) and 15 ml anhydrous methanol/H₂O (1:1) was added glyoxalic acid hydrate (1.01 g; 11 mmol) at room temperature, and the mixture was stirred for 1 h. The reaction mixture was then stirred with Na[BH₃(CN)] (0.63 g; 10 mmol) at room temperature for 22 h. The mixture was vigorously stirred. The solution was then rotary-evaporated to dryness and the residue vacuum dried. After evaporation the product was purified by a column chromatography on silica gel. Total yield of FA-Ia: 74 percent.

### Example 59

### General Procedures Preparations of active O—NSu N-carboxymethyl-ligands GA-Ia-GA-Id

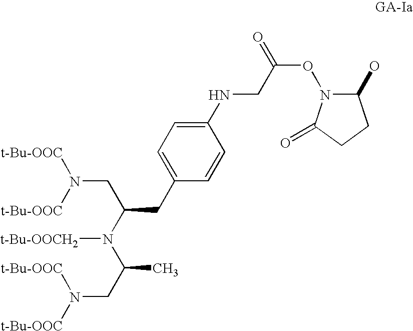

FA-Ia (238 mg, 280 μmol), N-hydroxysuccinimide (130 mg, 4 eq.) and 1-ethyl-3-[3-(N,N-dimethylaminopropyl)]-carbodiimidehydrochloride (EDC+HCl; 210 mg, 4 eq.) in DMF (1 mL) was stirred at 25° C. for 24 h to afford GA-Ia. The solution was then rotary-evaporated to dryness and the residue vacuum dried. After evaporation the product was purified by a column chromatography on silica gel (hexane/AcOEt). Total yield of GA-Ia: 76 percent.

### Example 60

### General Procedures Preparation of Precursors of GA-Ia-d with Derivate of d-Phe-Cys-Tyr-D-Trp-Lys(BOC)-Thr-Cys-L-threoninol (Disulfide Bond)

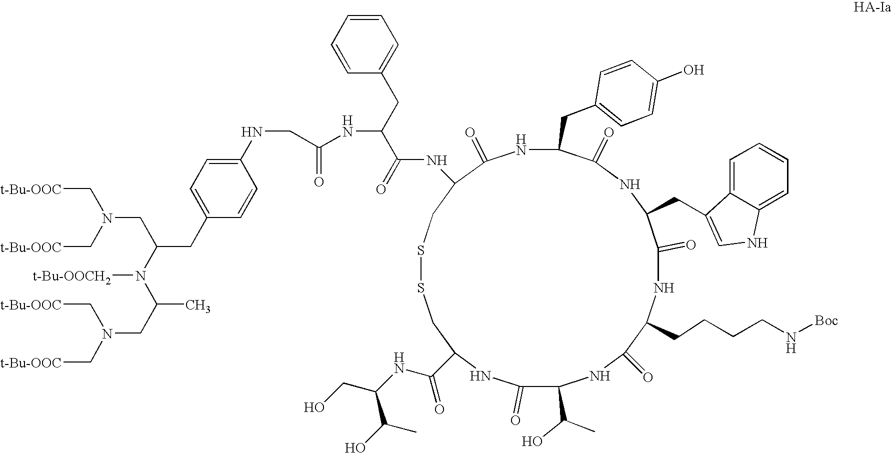

Compound GA-Ia (85 mg, 90 μmol, HATU (O-(7-azabenzotriazole-1-yl)-1,1,3,3-tetramethyluronium hexafluorophosphate) (34.2 μL, 90 μmol) and DIPEA ((N,N′-diisopropylethylamine) (15.3 μL, 90 μmol) were preincubated in DMF (1.5 mL). After 10 min. Tyr³-Lys⁵(BOC)-octreotide (87.9 mg, 75 micromol) and DIPEA (15.1 mL, 90 mmol) dissolved in DMF (1 mL) were added. Stirring was continued for 6 h to complete the reaction, then EtOAc (5 mL) and an aqueous solution of KHCO₃ (5%, 3 mL) were added. The organic layer was washed with KHCO₃ solution (5%, 3×2 mL) and the water layer with EtOAc (6×3 mL). The combined organic layers were washed with H₂O (4×3 mL). Evaporation afforded a crude product as a white solid which was not purified further. Total yield of HA-Ia: 82 percent

### Example 61

### General Procedures Preparation Conjugates of GA-Ia-d with Derivate of d-Phe-Cys-Tyr-D-Trp-Lys-Thr-Cys-L-threoninol (Disulfide Bond)

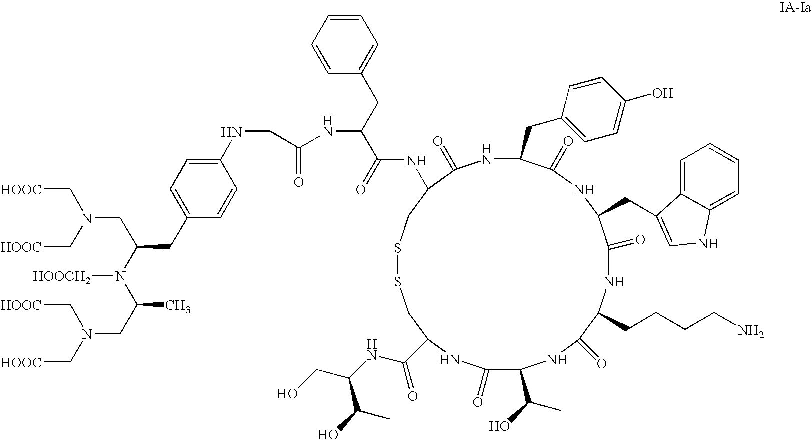

Compound HA-Ia (from Example 60—raw product) was dissolved in a deprotection mixture (TFA/thioanisole/H₂O, 92:6:2, v/v, 2 mL). After stirring for 4 h the solvent was removed by evaporation and the residue redissolved in H₂O (2 mL) and EtOAc (1 mL). The organic layer was washed with H₂O (3×0.5 mL) and the water layer with EtOAc (3×0.5 mL). The combined water layers were purified by RP-HPLC (Bio-Rad, 5 μmetr, C18, 1×25 cm, eluent: A: NH₄OAc (20 min, pH 5); B: AcCN; gradient: from 0-50% in 35 min at 1 mLmin⁻¹). Lyophilisation afforded the pure compound IA-Ia in 73% yield. purity (HPLC): >98%.

### Example 62

### General Procedures Preparation of Precursors of BA-Ia-d with Derivate of d-Phe-Cys-Tyr-D-Trp-Lys(BOC)-Thr-Cys-L-threoninol (Disulfide Bond)

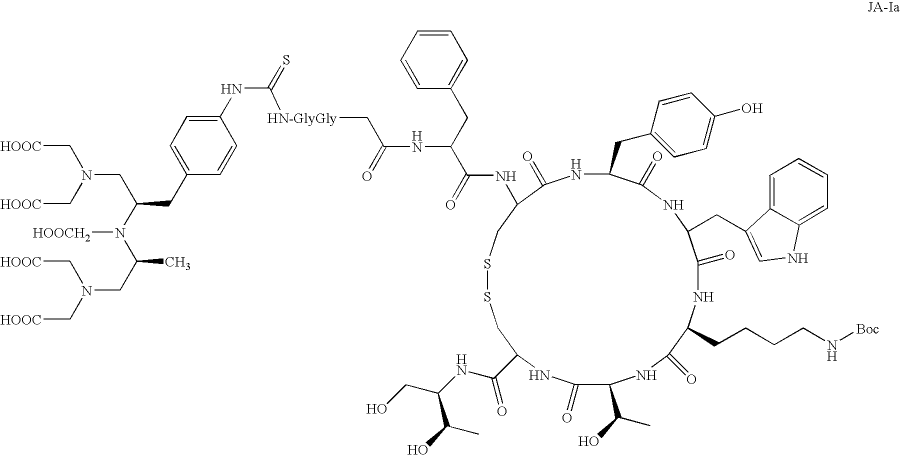

Compound BA-Ia (77 mg, 90 μmol; from Example 48) and DIPEA ((N,N′-diisopropylethylamine) (15.3 μL, 90 μmol) were dissolved in DMF (1.5 mL). After 3 min, Tyr³-Lys⁵ (BOC)-octreotide (87.9 mg, 75 micromol) and DIPEA (15.1 mL, 90 mmol) dissolved in DMF (1 mL) were added. Stirring was continued for 6 h to complete the reaction, then EtOAc (5 mL) and an aqueous solution of KHCO₃ (5%, 3 mL) were added. The organic layer was washed with KHCO₃ solution (5%, 3×2 mL) and the water layer with EtOAc (6×3 mL). The combined organic layers were washed with H₂O (4×3 mL). Evaporation afforded a crude product as a white solid which was not purified further. Total yield of JA-Ia: 76 percent

### Example 63

### General Procedures Preparation Conjugates of BA-Ia-d with Derivate of d-Phe-Cys-Tyr-D-Trp-Lys-Thr-Cys-L-threoninol (Disulfide Bond)

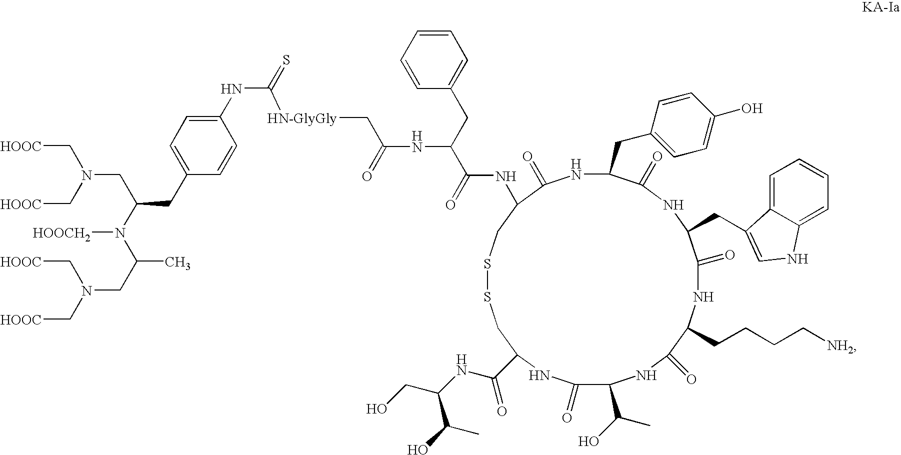

Compound JA-Ia (from Example 62—raw product) was dissolved in a deprotection mixture (TFA/thioanisole/H₂O, 92:6:2, v/v, 2 mL). After stirring for 4 h the solvent was removed by evaporation and the residue redissolved in H₂O (2 mL) and EtOAc (1 mL). The organic layer was washed with H₂O (3×0.5 mL) and the water layer with EtOAc (3×0.5 mL). The combined water layers were purified by RP-HPLC (Bio-Rad, 5 μmetr, C18, 1×25 cm, eluent A: NH₄OAc (20 min, pH 5); B: AcCN; gradient: from 0-50% in 35 min at 1 mLmin⁻¹). Lyophilisation afforded the pure compound KA-Ia in 81% yield. purity (HPLC): >97%.

### Example 64

### General Procedures Preparation of Precursors of YA-Ia-d with Derivate of Cys(Boc)-d-Phe-Cys-Tyr-D-Trp-Lys(BOC)-Thr-Cys-L-threoninol (Disulfide Bond)

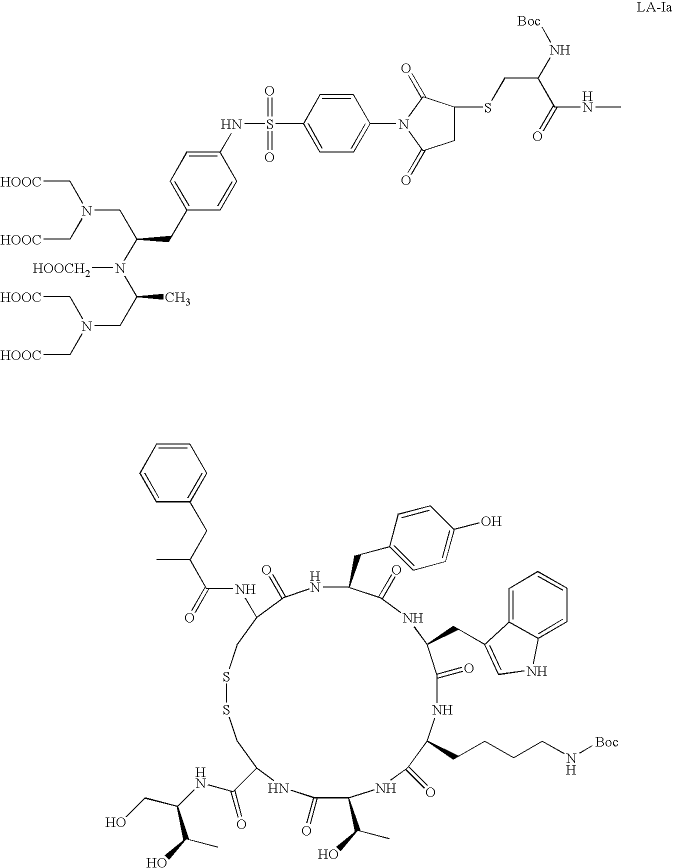

Compound YA-Ia (67.2 mg, 90 μmol, from Example 45), and DIPEA ((N,N′-diisopropylethylamine) (15.3 μL, 90 μmol) were dissolved in DMF (1.5 mL). After 10 min, Cys(Boc)-d-Phe-Cys-Tyr-D-Trp-Lys(BOC)-Thr-Cys-L-threoninol (disulfide bond) (75 micromol) and DIPEA (15.1 mL, 90 mmol) dissolved in DMF (1 mL) were added. Stirring was continued for 6 h to complete the reaction, then EtOAc (5 mL) and an aqueous solution of KHCO₃ (5%, 3 mL) were added. The organic layer was washed with KHCO₃ solution (5%, 3×2 mL) and the water layer with EtOAc (6×3 mL). The combined organic layers were washed with H₂O (4×3 mL). Evaporation afforded a crude product as a white solid which was not purified further. Total yield of LA-Ia: 64 percent.

### Example 65

### General Procedures Preparation Conjugates of YA-Ia-d with Derivate of Cys-d-Phe-Cys-Tyr-D-Trp-Lys-Thr-Cys-L-threoninol (Disulfide Bond)

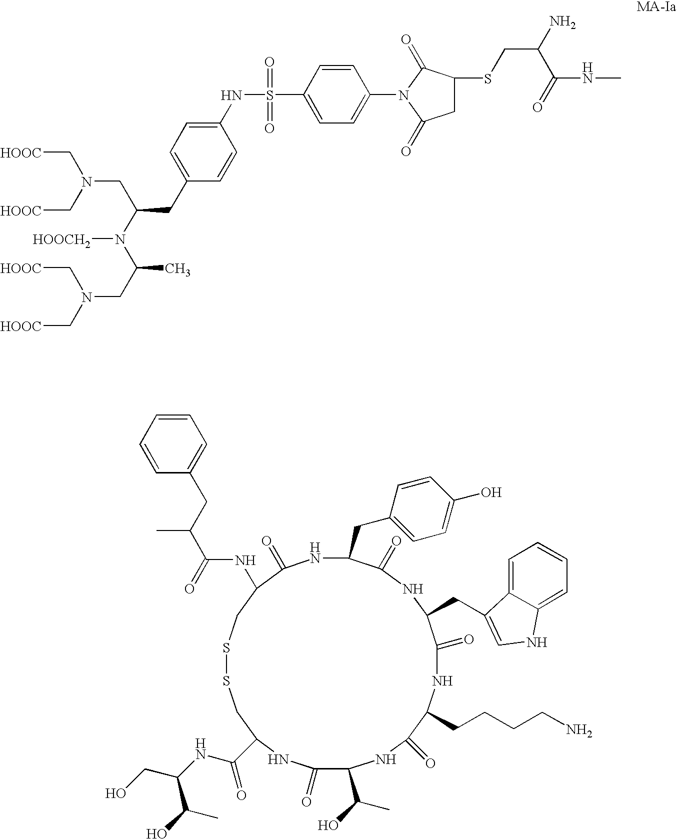

Compound LA-Ia (from Example 64—raw product) was dissolved in a deprotection mixture (TFA/thioanisole/H₂O, 92:6:2, v/v, 2 mL). After stirring for 4 h the solvent was removed by evaporation and the residue redissolved in H₂O (2 mL) and EtOAc (1 mL). The organic layer was washed with H₂O (3×0.5 mL) and the water layer with EtOAc (3×0.5 mL). The combined water layers were purified by RP-HPLC (Bio-Rad, 5 μmetr, C18, 1×25 cm, eluent: A: NH₄OAc (20 min, pH 5); B: AcCN; gradient from 0-50% in 35 min at 1 mLmin⁻¹). Lyophilisation afforded the pure compound MA-Ia in 72% yield. purity (HPLC): >96%.

### Example 66

### General Procedures for Conjugations of Peptides with SH Group

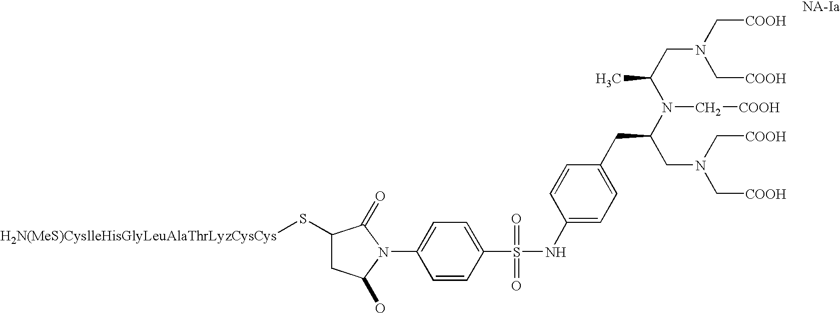

Conjugate NA-Ia were prepared by adding 3 molar excess of Y-Ia in DMSO (7 mg/ml) to SH-CysCysLyzThrAlaLeuGlyHisIleCys(SMe)NH₂ (10 mg/ml in borate-buffered saline (0.05 M, pH 8.5) prior to incubation at 37° C. for 20 hr. Conjugate was then purified by Sephadex G-50 column chromatography (1.8×40 cm) equilibrated and eluted with 0.1 M acetate buffer (pH 3.0). The respective conjugate fractions collected were subsequently concentrated to 5 mg/ml by ultrafiltration.

### Example 67

### General Procedures for Conjugations of Amino Acid

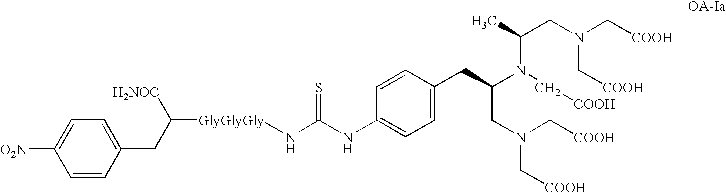

Conjugate OA-Ia were prepared by adding 3 molar excess of p-nitrophenylalanine amide in dimethylformamide (7 mg/ml) to BA-Ia (10 mg/ml) in borate-buffered saline (0.05 M, pH 8.5) prior to incubation at 37° C. for 20 hr. Conjugate was then purified by Sephadex G-50 column chromatography (1.8×40 cm) equilibrated and eluted with 0.1 M acetate buffer (pH 3.0). The product were purified by RP-HPLC (Bio-Rad, 5 μmetr, C18, 1×25 cm, eluent A: NH₄OAc (20 min, pH 5); B: AcCN; gradient from 0-50% in 35 min at 1 mLmin⁻¹). Lyophilisation afforded the pure compound OA-Ia in 69% yield. purity (HPLC): >98%.

### Example 68

### Preparation of Conjugate PA-Ia

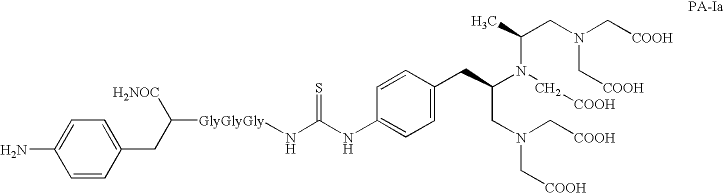

A solution 100 mg of OA-1a in H₂O (3 mL) and formic acid (3 mL) was hydrogenated at room temperature and 35 psi of H₂ over 10% palladium on carbon (0.21 g) for 25 h. The catalyst was then removed by filtration through Celite and the filtrate vas evaporated to dryness under vacuum. The resulting residue was lyophilised and afforded the pure compound PA-Ia in 69% yield. purity (HPLC): >97.5%.

### Example 69

### Preparation of Isothioconjugate RA-Ia

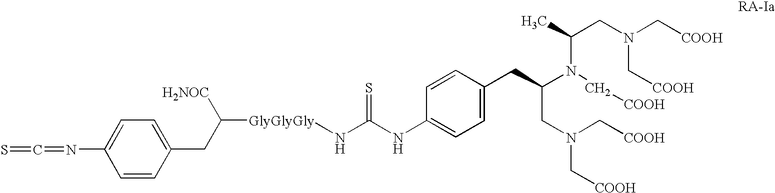

An 80% (v:v) solution of thiophosgene in CCl₄ (0.18 mL, 1.87 mmol) was added to a solution of 50 mg PA-Ia, in 3 M HCl (0.2 mL) and the resulting solution was vigorously stirred at room temperature for 6 h. The solvents and residual thiophosgene were then removed under vacuum in a fume hood, and the residue was dried further over P₂O₅ under vacuum. Total yield of RA-Ia: 99 percent.

### Example 70

### General Procedures Preparation of Precursors of SA-Ia-d with Derivate of d-Phe-Cys Tyr-D-Trp-Lys(BOC)-Thr-Cys-L-Thr (Disulfide Bond)

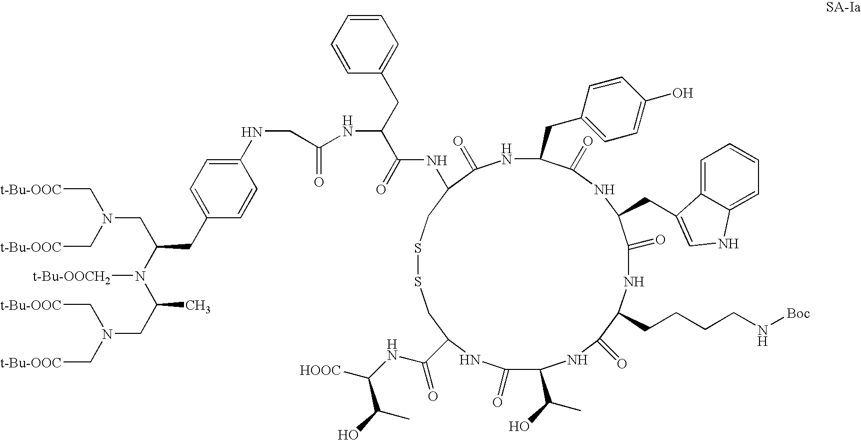

Compound GA-Ia (85 mg, 90 μmol), HATU (O-(7-azabenzotriazole-1-yl)-1,1,3,3-tetramethyluronium hexafluorophosphate) (34.2 μL, 90 μmol), and DIPEA ((N,N′-diisopropylethylamine) (15.3 μL, 90 μmol) were preincubated in DMF (1.5 mL). After 10 min, d-Phe-Cys-Tyr-D-Trp-Lys(BOC)-Thr-Cys-L-Thr (disulfide bond) (88 mg, 75 micromol) and DIPEA (15.1 mL, 90 mmol) dissolved in DMF (1 mL) were added. Stirring was continued for 6 h to complete the reaction, then EtOAc (5 mL) and an aqueous solution of KHCO₃ (5%, 3 mL) were added. The organic layer was washed with KHCO₃ solution (5%, 3×2 mL) and the water layer with EtOAc (6×3 mL). The combined organic layers were washed with H₂O (4×3 mL). Evaporation afforded a crude product as a white solid which was not purified further. Total yield of SA-Ia: 75 percent.

### Example 71

### General Procedures Preparation Conjugates of TA-Ia-d with Derivate of d-Phe-Cys-Tyr-D-Trp-Lys-Thr-Cys-L-Thr (Disulfide Bond)

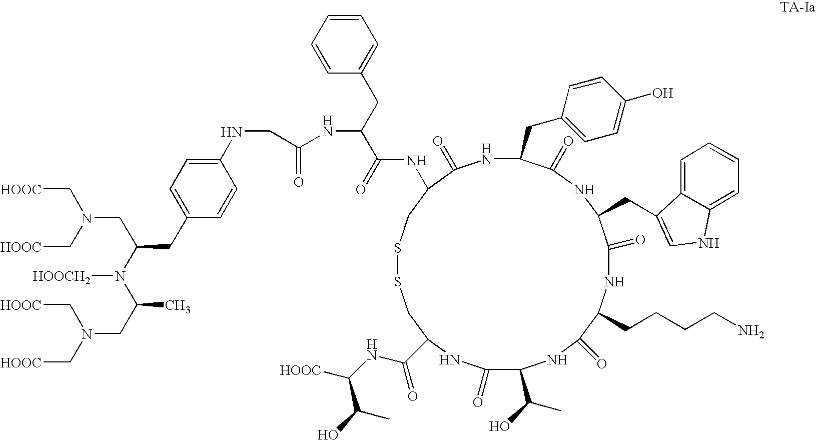

Compound SA-Ia (from Example 70—raw product) was dissolved in a deprotection mixture (TFA/thioanisole/H₂O, 92:6:2, v/v, 2 mL). After stirring for 4 h the solvent was removed by evaporation and the residue redissolved in H₂O (2 mL) and EtOAc (1 mL). The organic layer was washed with H₂O (3×0.5 mL) and the water layer with EtOAc (3×0.5 mL). The combined water layers were purified by RP-HPLC (Bio-Rad, 5 μmetr, C18, 1×25 cm, eluent A: NH₄OAc (20 min, pH 5); B: AcCN; gradient from 0-50% in 35 min at 1 mLmin⁻¹). Lyophilisation afforded the pure compound TA-Ia in 65% yield. purity (HPLC): >97%.

### Example 72

### General Procedures Preparation of Precursors of UA-Ia-d with Derivate of d-Phe-Cys-Tyr-D-Trp-Lys(BOC)-Thr-Cys-L-Thr (Disulfide Bond)

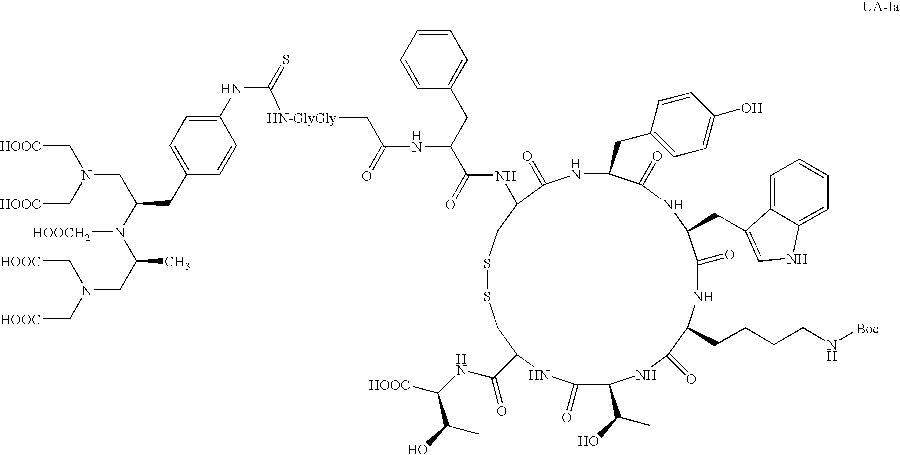

Compound BA-Ia (77 mg, 90 μmol; from Example 48) and DIPEA ((N,N′-diisopropylethylamine) (15.3 μL, 90 μmol) were dissolved in DMF (1.5 mL). After 3 min, d-Phe-Cys-Tyr-D-Trp-Lys(BOC)-Thr-Cys-L-Thr (disulfide bond) (88 mg, 75 micromol) and DIPEA (15.1 mL, 90 mmol) dissolved in DMF (1 mL) were added. Stirring was continued for 6 h to complete the reaction, then EtOAc (5 mL) and an aqueous solution of KHCO₃ (5%, 3 mL) were added. The organic layer was washed with KHCO₃ solution (5%, 3×2 mL) and the water layer with EtOAc (6×3 mL). The combined organic layers were washed with H₂O (4×3 mL). Evaporation afforded a crude product as a white solid which was not purified further. Total yield of UA-Ia: 75 percent.

### Example 73

### General Procedures Preparation Conjugates of VA-Ia-d with derivate of d-Phe-Cys-Tyr-D-Trp-Lys-Thr-Cys-L-Thr (Disulfide Bond)

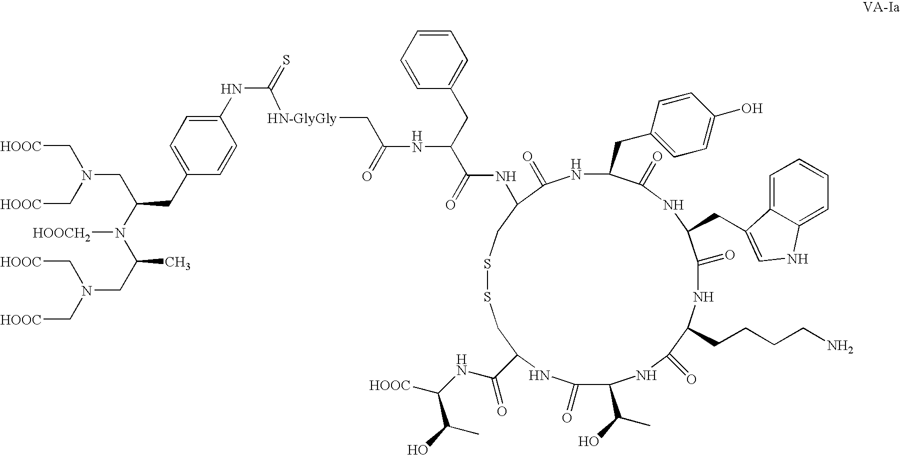

Compound UA-Ia (from Example 72—raw product) was dissolved in a deprotection mixture (TFA/thioanisole/H₂O, 92:6:2, v/v, 2 mL). After stirring for 4 h the solvent was removed by evaporation and the residue redissolved in H₂O (2 mL) and EtOAc (1 mL). The organic layer was washed with H₂O (3×0.5 mL) and the water layer with EtOAc (3×0.5 mL). The combined water layers were purified by RP-HPLC (Bio-Rad, 5 μmetr, C18, 1×25 cm, eluent A: NH₄OAc (20 min, pH 5); B: AcCN; gradient from 0-50% in 35 min at 1 mLmin⁻¹). Lyophilisation afforded the pure compound VA-Ia in 79% yield. purity (HPLC): >96%.

1-15. (canceled)

16. A pentapendant enantiomer-pure chelator represented by the structure (VII): 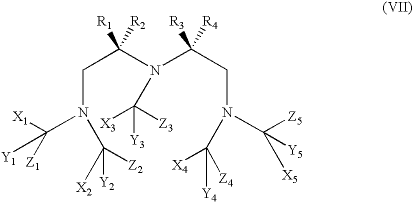
wherein
X₁-X₅, Y₁-Y₅, Z₁-Z₅ are each individually hydrogen, substituted or unsubstituted C₁-C₂₄ alkyl, C₂-C₂₄ alkenyl or cycloalkyl, substituted or unsubstituted aryl or heteroaryl, especially O-substituted or unsubstituted carboxyl, nitrile, N-substituted or unsubstituted carboxamide, formyl, N-hydroxyiminomethyl, independently O- and N-substituted or unsubstituted N-hydroxyaminocarbonyl, phosphonyl, phosphinyl, alkylphosphonyl, alkylphosphonyl, arylphosphonyl, arylphosphonyl forming pendants,
R₁, R₂, R₃, R₄ are groups forming an adequate enantiomer (R,R), (R,S), (S,R) or (S,S) wherein R₁, R₂, R₃, R₄ are independently hydrogen, substituted or unsubstituted C₁-C₂₄ alkyl, C₂-C₂₄ alkenyl or cycloalkyl, substituted or unsubstituted aryl or heteroaryl, especially 4-substituted benzyl of the structure (VIII) 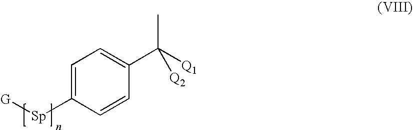
Q₁, Q₂ are each individually hydrogen, substituted or or unsubstituted C₁-C₂₄ alkyl, substituted or unsubstituted aryl or heteroaryl, substituted or unsubstituted carboxyl or N-substituted or unsubstituted carboxamide;
Sp is spacing group of the formula 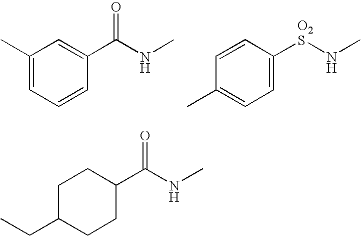
n is 0 or 1;
G is hydrogen, substituted or unsubstituted C₁-C₂₄ alkyl or C₂-C₂₄ alkenyl, N-substituted or unsubstituted amine, N-substituted or unsubstituted hydrazine, hydroxyl, O-alkylhydroxyl, O-acylhydroxyl, thiol, S-alkylthiol, O-substituted or unsubstituted carboxyl, N-substituted or unsubstituted carboxamide, isocayanate, isothiocyanate, carboxamidine, carbohydrazide, nitro, nitroso, formyl, formyl forming cyclic or uncyclic acetal, acetyl, 2-haloacetyl, halomethyl, hydroxymethyl or dihydroxyboronyl;
or is a linker of the formula
A
or
B
or
C
or
A-B-(C)ₐ
or
A₁-B-A₂-(C)ₐ
or
A₁-A₂-A₃-(C)ₐ
or
A₁-A₂-A₃-A₄-(C)ₐ
or
A₁-(A)_{β}-A₃-(C)ₐ
or
A₁-B₁-(A₂-B₂)_{γ}-A₃-B₃-(C)ₐ
wherein β, γ are each individually from 0 to 24; α is 0 or 1; wherein A₁, A₂, A₃,
A₄ are independently fragments of structure A; B₁, B₂ are independently fragments of structure B;
wherein A is a fragment of structure (IX) 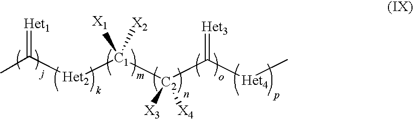
wherein j, k, m, n, o, p are each individually from 0 to 12; Het₁-Het₄ are independently O, S, NR_{Het}, wherein R_{Het} is hydrogen, substituted or unsubstituted C₁-C₁₂ alkyl, substituted or unsubstituted aryl;
X₁-X₄ are each individually hydrogen, substituted or unsubstituted primary C₁-C₁₂ alkyl or cycloalkyl, substituted or unsubstituted aryl, hydroxyl, alkoxyl, aryloxyl, halogen, substituted or unsubstituted amine, carboxyl, N-substituted or unsubstituted carboxamide, nitrile, alkoxycarbonyl;
or wherein X₁-X₄ can form mutually 5-membered and 6-membered saturated or unsaturated cycles, aromatic cycles and heterocycles; or X₁-X₄ can form mutually and each individually an oxo group, or a double and triple bond between C₁ and C₂;
wherein B is fragment of structure (X) 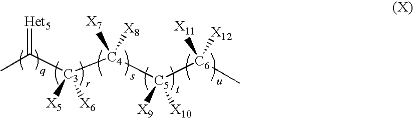
wherein q, r, s, t, u are each individually from 0 to 12; Het₅ is independently O, S, NR_{Het}, wherein R_{Het} is hydrogen, substituted or unsubstituted C₁-C₁₂ alkyl, substituted or unsubstituted aryl; X₅-X₁₂ are each individually hydrogen, primary substituted or unsubstituted C₁-C₁₂ or cycloalkyl, substituted or unsubstituted aryl, hydroxyl, alkoxyl, aryloxyl, halogen, substituted or unsubstituted amine, carboxyl, N-substituted or unsubstituted carboxamide, nitrile, alkoxycarbonyl, or X₅-X₁₂ can form mutually 5-membered and 6-membered saturated or unsaturated cycles, aromatic cycles and heterocycles, or X₅-X₁₂ can form mutually and each individually an oxo group, or one or two double and triple bonds between C₁, C₂, C₃ or C₄,
and wherein C is a reactive group, particularly a structural fragment selected from the group of hydroxyl, carboxyl, amino group, chloroacetyl, bromoacetyl group, iodoacetyl group, carbonyl chloride, carbonyl fluoride, carbonyl bromide, sulphonyl chloride, sulphonyl fluoride, sulphonyl bromide, sulphonyl arylsulphonate, sulphonyl alkylsulphonate, or an active ester, e.g. selected from the group: 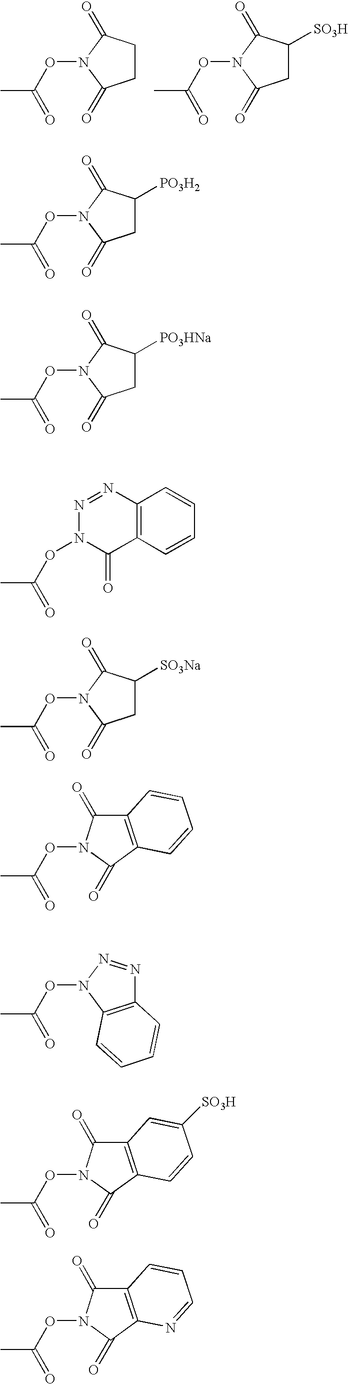 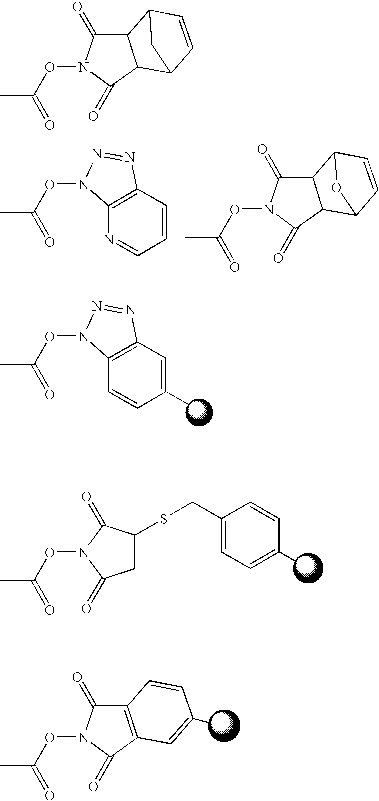
or from the group: 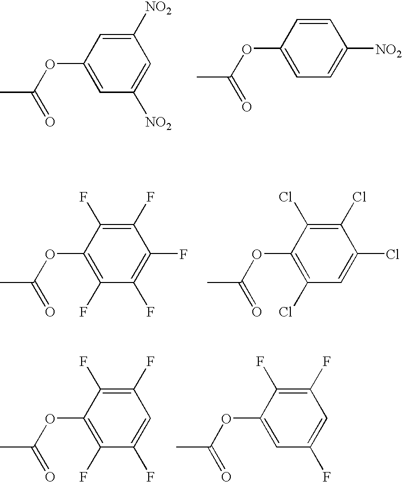
or a biologically active molecule, especially a biopolymer, which may be a natural substrate present in an organism or its synthetic analog, wherein the molecule preferably has biologic activity in a physiological function, especially in metabolic effect control or reproduction, wherein the biopolymer is preferably a polypeptide, or preferably comprises amino acids, wherein the biologically active molecule is preferably selected from the group consisting of:
antibodies, e.g. monoclonal antibodies (e.g. antiCD33, antiCD25, antiCD66), antibody fragments, polyclonal antibodies, minibodies, somatostatin and derivatives thereof, IGF-1 (somatomedin) and derivatives thereof, IGF-2, IGF-protein-3, somatostatin-biotin derivatives, tumor-specific proteins and synthetic agents, vascular endothelial growth factor, myoglobins, apomyoglobins, neurotransmitter peptides, octreotide, lanreotide, Somatuline, vapreotide, tumor necrosis factors and peptides that accumulate in inflamed tissues.

17. Process for the production of compounds according to claim 16 based on reaction of enantiomer-pure amine of the structure (XI) 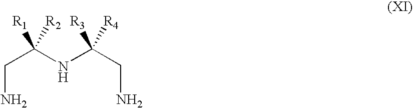
wherein
R₁, R₂, R₃, R₄ are groups forming an adequate enantiomer (R,R), (R,S), (S,R) or (S,S), wherein R₁, R₂, R₃, R₄ are independently hydrogen, substituted or unsubstituted C₁-C₂₄ alkyl, C₂-C₂₄ alkenyl or cycloalkyl, substituted or unsubstituted aryl or heteroaryl, especially 4-substituted benzyl of the structure (VIII) as defined in claim 16,
wherein
Q₁, Q₂ are each individually hydrogen, substituted or unsubstituted C₁-C₂₄ alkyl, substituted or unsubstituted aryl oder heteroaryl, substituted or unsubstituted carboxyl, or N-substituted or unsubstituted carboxamide;
Sp is spacing group of the formula 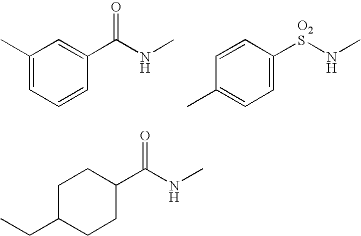
n is 0 or 1;
G is hydrogen, C₁-C₂₄ alkenyl, N-substituted or unsubstituted amine, N-substituted or unsubstituted hydrazine, hydroxyl, O-alkylhydroxyl, O-acylhydroxyl, thiol, S-alkylthiol, O-substituted or unsubstituted carboxyl, N-substituted or unsubstituted carboxamide, isocyanate, isothiocyanate, carboxamidine, carbohydrazide, nitro, nitroso, formyl, formyl forming cyclic or uncyclic acetal, acetyl, 2-haloacetyl, halomethyl, hydroxymethyl or dihydroxyboronyl;
or is a linker of the formula
A
or
B
or
C
or
A-B-(C)ₐ
or
A₁-B-A₂-(C)ₐ
or
A₁-A₂-A₃-(C)ₐ
or
A₁-A₂-A₃-A₄-(C)ₐ
or
A₁-(A)_{β}-A₃-(C)ₐ
or
A₁-B₁-(A₂-B₂)_{γ}-A₃-B₃-(C)ₐ
wherein β, γ are each individually from 0 to 24; α is 0 or 1; wherein A₁, A₂, A₃, A₄ are independently fragments of structure A; B₁, B₂ are independently fragments of structure B;
wherein A is a fragment of structure (IX) as shown in claim 16,
wherein j, k, m, n, o, p are each individually from 0 to 12; Het₁-Het₄ are independently O, S, NR_{Het}, wherein R_{Het} is hydrogen, substituted or unsubstituted C₁-C₁₂ alkyl, substituted or unsubstituted aryl;
X₁-X₄ are each individually hydrogen, substituted or unsubstituted primary C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl; or X₁-X₄ can form mutually 5-membered and 6-membered saturated or unsaturated cycles, aromatic cycles and heterocycles; or X₁-X₄ can form mutually and each individually an oxo group, or a double and triple bond between C₁ and C₂;
wherein B is a fragment of structure (X) as shown in claim 16,
wherein q, r, s, t, u are each individually from 0 to 12; Het₅ is independently O, S, NR_{Het}, wherein R_{Het} is hydrogen, substituted or unsubstituted C₁-C₁₂ alkyl, substituted or unsubstituted aryl; X₅-X₁₂ are each individually hydrogen, substituted or unsubstituted primary C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl or X₅-X₁₂ can form mutually 5-membered and 6-membered saturated or unsaturated cycles, aromatic cycles and heterocycles or X₅-X₁₂ can form mutually and each individually an oxo group, or one or two double and triple bonds between C₁, C₂, C₃ or C₄,
and wherein C is a reactive group, particularly a structural fragment selected from the group of hydroxyl, carboxyl, amino group, chloroacetyl, bromoacetyl group, iodoacetyl group, carbonyl chloride, carbonyl fluoride, carbonyl bromide, sulphonyl chloride, sulphonyl fluoride, sulphonyl bromide, sulphonyl arylsulphonate, sulphonyl alkylsulphonate, or an active ester, e.g. selected from the group: 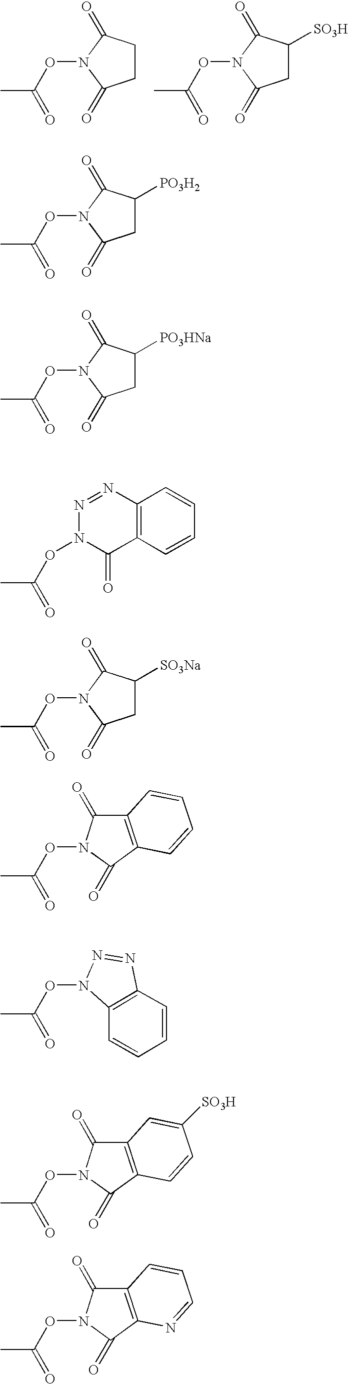 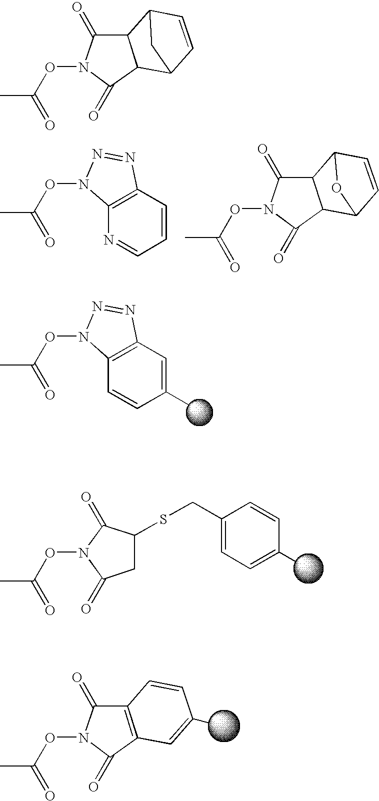
or from the group: 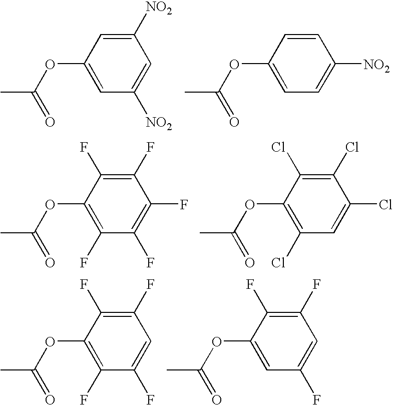
or a biologically active molecule, especially a biopolymer, which may be a natural substrate present in an organism or its synthetic analog, wherein the molecule preferably has biologic activity in a physiological function, especially in metabolic effect control or reproduction, wherein the biopolymer is preferably a polypeptide, or preferably comprises amino acids, wherein the biologically active molecule is preferably selected from the group consisting of:
antibodies, e.g. monoclonal antibodies (e.g. antiCD33, antiCD25, antiCD66), antibody fragments, polyclonal antibodies, minibodies, somatostatin and derivatives thereof, IGF-1 (somatomedin) and derivatives thereof, IGF-2, IGF-protein-3, somatostatin-biotin derivatives, tumor-specific proteins and synthetic agents, vascular endothelial growth factor, myoglobins, apomyoglobins, neurotransmitter peptides, octreotide, lanreotide, Somatuline, vapreotide, tumor necrosis factors and peptides that accumulate in inflamed tissues,
by a carboxyalkylation or by a phosphonoalkylation or by a phosphinoalkylation with an agent of the structure (XII) 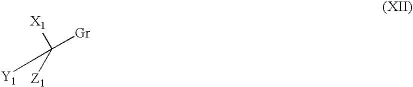
wherein
X₁-X₅, Y₁-Y₅, Z₁-Z₅ are each individually hydrogen, substituted or unsubstituted C₁-C₂₄ alkyl, C₁-C₂₄ alkenyl or cycloalkyl, substituted or unsubstituted aryl or heteroaryl, especially O-substituted or unsubstituted carboxyl, nitrile, N-substituted or unsubstituted carboxamide, formyl, N-hydroxyiminomethyl, alkoxycarbonyl, aryloxycarbonyl, independently O- and N-substituted or unsubstituted N-hydroxyaminocarbonyl, phosphonyl, phosphinyl, alkylphosphonyl, alkylphosphonyl, arylphosphonyl, arylphosphonyl and just one or two substituents from X₁-X₅, Y₁-Y₅, Z₁-Z₅ are each individually carboxyl, nitrile, N-substituted or unsubstituted carboxamide, formyl, alkoxycarbonyl, aryloxycarbonyl, N-hydroxyiminomethyl or independently O- and N-substituted or unsubstituted N-hydroxyaminocarbonyl, phosphonyl, phosphinyl, alkylphosphonyl, alkylphosphonyl, arylphosphonyl or arylphosphonyl,
wherein
Gr is halogen, hydroxyl, alkoxyl, aryloxyl, oxonium, substituted or unsubstituted amine, substituted or unsubstituted ammonium, sulphonyl, sulphonyloxy, O-acyloxyl, arylsulphonyloxy, halogen especially bromine, chlorine, iodine, tosyloxy, mesyloxy, triflyloxy, benzoyloxy, methoxycarbonyloxy, perfluoracetyloxy, trimethylammonium, diethyloxonium, 1-benztriazolyloxyl, trialkylsilyloxyl, benzyloxycarbonyloxy, tert.butyloxycarbonyloxyl, N-phthalimidyloxy, 1-imidazolyloxy, N-succinimidyloxyl, N-phthalimidyloxy,
or wherein the agent (XII) is generated in situ from a two- or three-part reaction system, e.g. from hydrogen cyanide and formaldehyde; alkaline cyanide, formaldehyde and a mineral acid; formaldehyde and methyl(4-nitrobenzyl)oxophosphorane; formaldehyde and methylphosphinic acid; formaldehyde and diethyl phosphonate; formaldehyde diethylacetal and 4,5-diphenyl-1,3,2λ⁵-dioxaphospholan-2-one,
under conditions of general nucleophilic substitution, especially under conditions of phase-transfer catalysis, e.g. in an aprotic polar solvent or a mixture of such solvents (such as dimethylformamide or dimethylacetamide or acetonitrile, dimethylsulphoxide or sulpholane or hexamethylphosphortriamide) or a mixture with at least one protic solvent, e.g. in a micellar medium, in solid-phase (for example with bonded amine (XI) on anex), with or without microwave irradiation, with or without ultrasonic irradiation, under conditions of high pressure (for example in autoclave), in aqueous or nonaqueous phase in presence of pH-buffer, in milieu of water-free solvents with or without presence of base (e.g. amines, aldimines, carbonates, fluorides, thioethers), especially a strong base with low nucleophily (e.g. N-ethyl-N,N-diisopropylamine (Hünings base), N-methyl-N,N-dicyclohexylamine, N-methyl-N,N-diisopropylamine, N,N,N′,N′-tetramethyl-1,8-naphtalenediamine), with enzymatic catalysis, in presence of a dehydrating agent or an agent reacting with protogenic product reaction or in presence of a Lewis acid (e.g. ZnCl₂, BF₃.Et₂O, SiCl₄).

18. The process according to claim 17 which is performed in a temperature range of −78° C.-325° C.

19. The process according to claim 17 which is carried out from a period of 15 seconds to ten days.

20. The process for reacting of compounds represented by the structure (VII) according to claim 16,
wherein
R₁, R₂, R₃, R₄ are groups forming an adequate enantiomer (R,R), (R,S), (S,R) or (S,S), wherein R₁, R₂, R₃, R₄ are independently hydrogen, substituted or unsubstituted C₁-C₂₄ alkyl, C₂-C₂₄ alkenyl or cycloalkyl, substituted or unsubstituted aryl or heteroaryl, especially 4-substituted benzyl of the structure (VIII) as shown in claim 16,
wherein
Q₁, Q₂ are each individually hydrogen, substituted or unsubstituted C₁-C₂₄ alkyl, substituted or unsubstituted aryl or heteroaryl, substituted or unsubstituted carboxyl, N-substituted or unsubstituted carboxamide;
Sp is spacing group of the formula 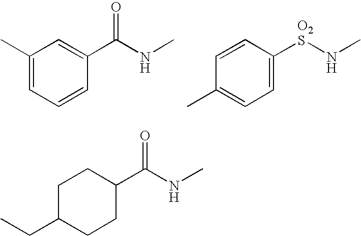
n is 0 or 1;
G is a linker of the formula
A-B-(C)ₐ
or
A₁-B-A₂-(C)ₐ
or
A₁-A₂-A₃-(C)ₐ
or
A₁-A₂-A₃-A₄-(C)ₐ
or
A₁-(A)_{β}-A₃-(C)ₐ
or
A₁-B₁-(A₂-B₂)_{γ}-A₃-B₃-(C)ₐ
wherein β, γ are each individually from 0 to 24; α is 1; wherein A₁, A₂, A₃, A₄ are independently fragments of structure A; B₁, B₂ are independently fragments of structure B;
wherein A is a fragment of structure (IX) as shown in claim 16,
wherein j, k, m, n, o, p are each individually from 0 to 12; Het₁-Het₄ are independently O, S, NR_{Het}, wherein R_{Het} is hydrogen, substituted or unsubstituted C₁-C₁₂ alkyl or aryl;
X₁-X₄ are each individually hydrogen, primary substituted or unsubstituted C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl; X₁-X₄ can form mutually 5-membered and 6-membered saturated or unsaturated cycles, aromatic cycles and heterocycles; or X₁-X₄ can form mutually and each individually an oxo group, or a double and triple bond between C₁ and C₂;
wherein B is fragment of structure (X) as shown in claim 16,
wherein q, r, s, t, u are each individually from 0 to 12; Het₅ is independently O, S, NR_{Het}, wherein R_{Het} is hydrogen, substituted or unsubstituted C₁-C₁₂ alkyl, substituted or unsubstituted aryl; X₅-X₁₂ are each individually hydrogen, substituted or unsubstituted C₁-C₁₂ alkyl or cycloalkyl, substituted or unsubstituted aryl, hydroxyl, alkoxy, aryloxyl, halogen, substituted or unsubstituted amine, carboxyl, N-substituted or unsubstituted carboxamide, nitrile, alkoxycarbonyl or X₅-X₁₂ can forms mutually 5-membered and 6-membered saturated or unsaturated cycles, aromatic cycles and heterocycles; or X₅-X₁₂ can form mutually and each individually an oxo group or one or two double and triple bonds between C₁, C₂, C₃ or C₄,
and wherein C is a reactive group, particularly a structural fragment selected from the group of hydroxyl, carboxyl, amino group, isothiocyanate, chloroacetyl, bromoacetyl group, iodoacetyl group, carbonyl chloride, carbonyl fluoride, carbonyl bromide, sulphonyl chloride, sulphonyl fluoride, sulphonyl bromide, sulphonyl arylsulphonate, sulphonyl alkylsulphonate, or an active ester, e.g. selected from the group: 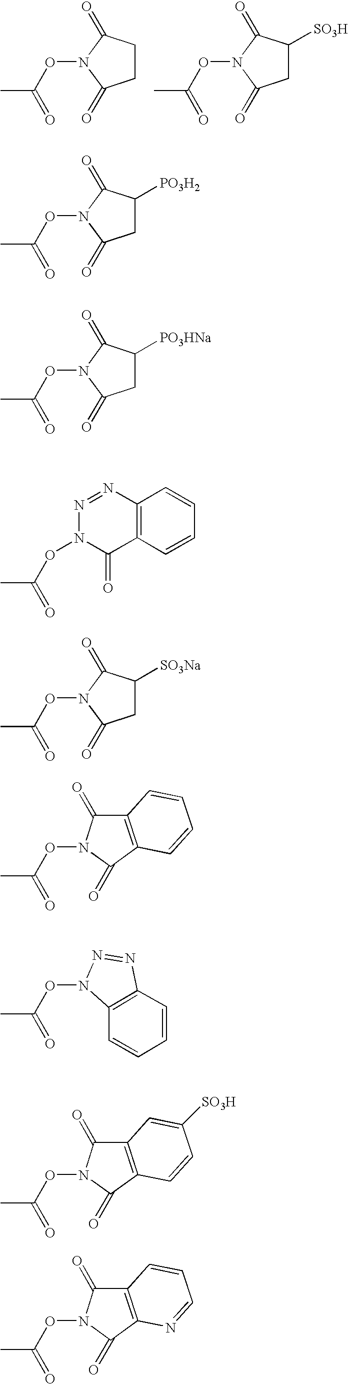 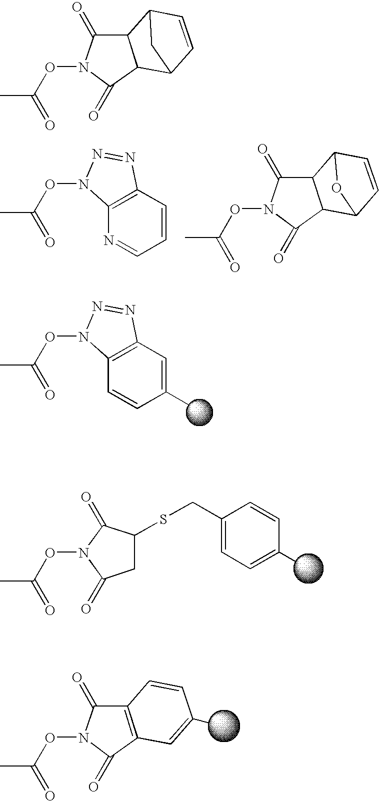
or from the group: 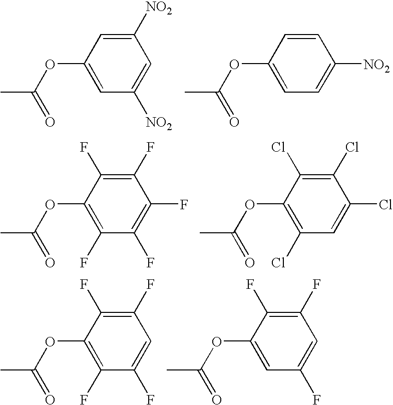
with biologically active molecule, especially a biopolymer by covalent binding, especially a biopolymer, which may be a natural substrate present in an organism or its synthetic analog, wherein the molecule preferably has biologic activity in a physiological function, especially in metabolic effect control or reproduction, wherein the biopolymer is preferably a polypeptide, or preferably comprises amino acids, wherein the biologically active molecule is preferably selected from the group consisting of:
antibodies, e.g. monoclonal antibodies (e.g. antiCD33, antiCD25, antiCD66), antibody fragments, polyclonal antibodies, minibodies, somatostatin and derivatives thereof, IGF-1 (somatomedin) and derivatives thereof, IGF-2, IGF-protein-3, somatostatin-biotin derivatives, tumor-specific proteins and synthetic agents, vascular endothelial growth factor, myoglobins, apomyoglobins, neurotransmitter peptides, octreotide, lanreotide, Somatuline, vapreotide, tumor necrosis factors and peptides that accumulate in inflamed tissues.

21. The compound according to claim 16 having a regulated and controlled biodistribution.

22. A complex of a pendapendent enantiomer-pure chelator according to claim 16 with a chelant, particularly a NMR-active or radioactive moiety.

23. A pharmaceutical composition that contains at least one physiologically active compound according to claim 16.

24. The pharmaceutical composition that contains at least one complex according to claim 23.

25. The composition according to claim 24 which is a diagnostic composition.

26. The composition according to claim 24 which is a therapeutic composition.

27. Use of a compound according to claim 16 for the manufacture of agents for NMR diagnosis and radiodiagnosis.

28. Use of a complex according to claim 22 for the manufacture of agents for NMR diagnosis and radiodiagnosis.

29. Use of a compound according to claim 16 for the manufacture of agents for radiotherapy.

30. Use of a complex according to claim 22 for the manufacture of agents for radiotherapy.
